(19) United States
(12) Patent Application Publication
(10) Pub. No.: US 2022/0275088 A1
(43) Pub. Date: Sep. 1, 2022

(54) ANTIBODIES AGAINST PD-1 AND METHODS OF USE THEREOF

(71) Applicant: Dana-Farber Cancer Institute, Inc., Boston, MA (US)

(72) Inventors: Wayne A. Marasco, Wellesley, MA (US); Matthew Chang, Brookline, MA (US); Quan Karen Zhu, Southborough, MA (US)

(21) Appl. No.: 17/618,360

(22) PCT Filed: Jun. 15, 2020
(86) PCT No.: PCT/US2020/037781

§ 371 Date: Dec. 10, 2021

Related U.S. Application Data
(60) Provisional application No. 62/861,643, filed on Jun. 14, 2019.

(51) Int. Cl. C07K 16/28

(52) U.S. Cl. CPC C07K 16/2818; C07K 2317/565; C07K 2317/622; C07K 2317/92; C07K 2317/31; C07K 2317/76; C07K 2317/33

(57) ABSTRACT

The present invention is directed to human monoclonal antibodies that bind to the cell-surface receptor, PD-1 (programmed death 1). The antibodies can be used to treat cancer and chronic viral infections.

This application is an International Application, which claims the benefit of priority from U.S. provisional patent application No. 62/861,643 filed on Jun. 14, 2019, the entire contents of which is incorporated herein by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to antibodies against PD-1 and methods of use thereof.

### BACKGROUND OF THE INVENTION

Programmed cell death-1 (PD-1), is a cell surface membrane protein of the immunoglobulin superfamily. This protein is expressed in pro-B-cells and is thought to play a role in their differentiation. A member of the CD28 family, PD-1 is upregulated on activated T cells, B cells, and monocytes. PD-1 has two identified ligands in the B7 family, PD-L1 (programmed cell death-1 ligand 1; also known as cluster of differentiation 274 (CD274) or B7 homolog 1 (B7-H1)) and PD-L2. PD-L1 is a 40 kDa type I transmembrane protein. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2. While PD-L2 expression tends to be more restricted, found primarily on activated antigen-presenting cells (APCs), PD-L1 expression is more widespread, including cells of hematopoietic lineage (including activated T cells, B cells, monocytes, dendritic cells and macrophages) and peripheral nonlymphoid tissues (including heart, skeletal, muscle, placenta, lung, kidney and liver tissues). The widespread expression of PD-L1 indicates its significant role in regulating PD-1/PD-L1-mediated peripheral tolerance.

### SUMMARY OF THE INVENTION

The present invention provides for PD-1 antibody compositions and methods of use of same.

An aspect of the invention is directed to an isolated monoclonal antibody or antigen-binding fragment thereof that binds to human Programmed cell death 1 (PD-1) protein. In one embodiment, the isolated monoclonal PD-1 antibody or antigen-binding fragment thereof comprises a heavy chain, light chain, or a combination thereof. In some embodiments, the heavy chain comprises a CDR1 comprising G-(X₁)—TF—(X₂X₃)—Y—(X₄) (SEQ ID NO: 81), G-(X₅)—TF—(X₆X₇X₈)-A (SEQ ID NO: 82), GDSVSSDNYF (SEQ ID NO: 43), or GYTFNRFG (SEQ ID NO: 55); a CDR2 comprising ISWNSGSI (SEQ ID NO: 19), IYPDDSDT (SEQ ID NO: 33), VYYNGNT (SEQ ID NO: 45), TNPYNGNT (SEQ ID NO: 57), or ISYDGSNK (SEQ ID NO: 69); a CDR3 comprising ASDYGDKYYYYGMDV (SEQ ID NO: 21), AFWGASGAPVNGFDI (SEQ ID NO: 35), ATETPPTSYFNSGPFDS (SEQ ID NO: 47), ARVVAVNGMDV (SEQ ID NO: 59), ASQTVAGSDY (SEQ ID NO: 71), or ASDYGDKYSYYGMDV (SEQ ID NO: 79); or a combination of CDRs thereof. In other embodiments, the light chain comprises a CDR1 comprising SSNIGSNT (SEQ ID NO: 24), SSNIGAGYV (SEQ ID NO: 37), SNNVGAHG (SEQ ID NO: 49), SGSIAAYY (SEQ ID NO: 61), or NIGSKS (SEQ ID NO: 73); a CDR2 comprising (X₉)-DN (SEQ ID NO: 83), (X₁₀)—NN (SEQ ID NO: 84), or DDS (SEQ ID NO: 75); a CDR3 comprising AAWDGGLNGRGV (SEQ ID NO: 28), AAWDDSLNAPV (SEQ ID NO: 41), SSWDSSLSGYV (SEQ ID NO: 53), QSYDSSNLWV (SEQ ID NO: 65), or QVWHSVSDQGV (SEQ ID NO: 77); or a combination of CDRs thereof. In some embodiments, the isolated monoclonal PD-1 antibody or antigen-binding fragment thereof comprises a heavy chain and a light chain comprising the CDRs described herein. In further embodiments, the isolated monoclonal PD-1 antibody or antigen-binding fragment thereof is fully human or humanized. In further embodiments, the isolated monoclonal PD-1 antibody or antigen-binding fragment thereof is monospecific, bispecific, or multispecific. In further embodiments, the isolated monoclonal PD-1 antibody or antigen-binding fragment thereof is a single chain antibody. In other embodiments, the isolated monoclonal PD-1 antibody or antigen-binding fragment thereof has a binding affinity of at least 1.0×10⁻⁹ M. In other embodiments, the isolated monoclonal PD-1 antibody or antigen-binding fragment thereof further comprises a heavy chain constant region, a light chain constant region, an Fc region, or a combination thereof. In some embodiments, the X₁, X₄, X₅ or X₈ amino acid residue of a CDR from the isolated monoclonal PD-1 antibody or antigen-binding fragment thereof is a non-polar amino acid residue. In some embodiments, the X₁, X₄, X₅ or X₈ amino acid residue of a CDR from the isolated monoclonal PD-1 antibody or antigen-binding fragment thereof is tyrosine (Y), phenylalanine (F), or alanine (A). In some embodiments, the X₂, X₃, X₄, X₆, X₇ or X₈ amino acid residue of a CDR from the isolated monoclonal PD-1 antibody or antigen-binding fragment thereof is a polar amino acid residue. In some embodiments, the X₂, X₃, X₄, X₆, X₇ or X₈ amino acid residue of a CDR from the isolated monoclonal PD-1 antibody or antigen-binding fragment thereof is aspartate (D), threonine (T), serine (S), or tryptophan (W). In other embodiments, the X₁ amino acid residue of a CDR from the isolated monoclonal PD-1 antibody or antigen-binding fragment thereof is tyrosine (Y) or phenylalanine (F). In other embodiments, the X₂ amino acid residue of a CDR from the isolated monoclonal PD-1 antibody or antigen-binding fragment thereof is aspartate (D), threonine (T), or serine (S). In other embodiments, the X₃ amino acid residue of a CDR from the isolated monoclonal PD-1 antibody or antigen-binding fragment thereof is aspartate (D), threonine (T), or serine (S). In other embodiments, the X₄ amino acid residue of a CDR from the isolated monoclonal PD-1 antibody or antigen-binding fragment thereof is alanine (A), or tryptophan (W). In other embodiments, the X₅ amino acid residue of a CDR from the isolated monoclonal PD-1 antibody or antigen-binding fragment thereof is phenylalanine (F) or tyrosine (Y). In other embodiments, the X₆ amino acid residue of a CDR from the isolated monoclonal PD-1 antibody or antigen-binding fragment thereof is aspartate (D), or serine (S). In other embodiments, the X₇ amino acid residue of a CDR from the isolated monoclonal PD-1 antibody or antigen-binding fragment thereof is aspartate (D), or serine (S). In other embodiments, the X₈ amino acid residue of a CDR from the isolated monoclonal PD-1 antibody or antigen-binding fragment thereof is phenylalanine (F) or tyrosine (Y). In other embodiments, the X₉ amino acid residue of a CDR from the isolated monoclonal PD-1 antibody or antigen-binding fragment thereof is a polar hydrophilic amino acid residue. In other embodiments, the X₉ amino acid residue of a CDR from the isolated monoclonal PD-1 antibody or antigen-binding fragment thereof is glutamate (E), asparagine (N), or aspartate (D). In other embodiments, the X₁₀ amino acid residue of a CDR from the isolated monoclonal PD-1 antibody or antigen-binding fragment thereof is a polar hydrophilic amino acid residue. In other embodiments, the X₁₀ amino acid residue of a CDR from the isolated monoclonal PD-1 antibody or antigen-binding fragment thereof is serine (S) or arginine (R).

An aspect of the invention is directed to antibody composition comprising at least one antibody, wherein the at least one antibody comprises two heavy chains and two light chains. In some embodiments, the heavy chain CDRs are identical to reference germline CDRs found between residues 27 and 38, residues 56 and 65, and residues 105 and 119 according to IMGT numbering of SEQ ID NO: 1; or between residues 27 and 38, residues 56 and 65, and residues 105 and 119 according to IMGT numbering of SEQ ID NO: 3; or between residues 27 and 38, residues 56 and 65, and residues 105 and 121 according to IMGT numbering of SEQ ID NO: 5; or between residues 27 and 38, residues 56 and 65, and residues 105 and 115 according to IMGT numbering of SEQ ID NO: 7; or between residues 27 and 38, residues 56 and 65, and residues 105 and 114 according to IMGT numbering of SEQ ID NO: 9; or between residues 27 and 38, residues 56 and 65, and residues 105 and 119 according to IMGT numbering of SEQ ID NO: 12 [e.g., the HL-14 MUTANT described herein]; or between residues 27 and 38, residues 56 and 65, and residues 105 and 119 according to IMGT numbering of SEQ ID NO: 13 [e.g., HLkin-1 MUTANT described herein]; or between residues 27 and 38, residues 56 and 65, and residues 105 and 119 according to IMGT numbering of SEQ ID NO: 15 [e.g., the mut-3 MUTANT described herein]; except that at least one of the heavy chain CDRs differs by a single amino acid substitution relative to its reference CDR. In some embodiments, the light chain CDRs are identical to reference germline CDRs found between residues 27 and 38, residues 56 and 65, and residues 105 and 116 according to IMGT numbering of SEQ ID NO: 2; or between residues 27 and 38, residues 56 and 65, and residues 105 and 115 according to IMGT numbering of SEQ ID NO: 4; or between residues 27 and 38, residues 56 and 65, and residues 105 and 115 according to IMGT numbering of SEQ ID NO: 6; or between residues 27 and 38, residues 56 and 65, and residues 105 and 114 according to IMGT numbering of SEQ ID NO: 8; or between residues 27 and 38, residues 56 and 65, and residues 105 and 115 according to IMGT numbering of SEQ ID NO: 10; or between residues 27 and 38, residues 56 and 65, and residues 105 and 116 according to IMGT numbering of SEQ ID NO: 11 [e.g., the HL-7 mutant described herein]; except that at least one of the light chain CDRs differs by a single amino acid substitution relative to its reference CDR. In some embodiments, the antibody composition binds to an epitope that comprises amino residues within the PD-1 face generated by the FCC′ strands but which do not contact the C′D loop of PD-1 comprising non-contiguous amino acids in SEQ ID NO: XX.

An aspect of the invention is directed to an isolated antibody or fragment thereof that binds to human Programmed cell death 1 (PD-1) protein. In one embodiment, the isolated antibody or fragment thereof that binds to PD-1 comprises a VH CDR1 comprising the amino acid sequence of SEQ ID NO: 17, a VH CDR2 comprising the amino acid sequence of SEQ ID NO: 19, a VH CDR3 comprising the amino acid sequence of SEQ ID NO: 21, a VL CDR1 comprising the amino acid sequence of SEQ ID NO: 24, a VL CDR2 comprising the amino acid sequence of SEQ ID NO: 26, and a VL CDR3 comprising the amino acid sequence of SEQ ID NO: 28. In one embodiment, the isolated antibody or fragment thereof that binds to PD-1 comprises a VH CDR1 comprising the amino acid sequence of SEQ ID NO: 31, a VH CDR2 comprising the amino acid sequence of SEQ ID NO: 33, a VH CDR3 comprising the amino acid sequence of SEQ ID NO: 35, a VL CDR1 comprising the amino acid sequence of SEQ ID NO: 37, a VL CDR2 comprising the amino acid sequence of SEQ ID NO: 39, and a VL CDR3 comprising the amino acid sequence of SEQ ID NO: 41. In one embodiment, the isolated antibody or fragment thereof that binds to PD-1 comprises a VH CDR1 comprising the amino acid sequence of SEQ ID NO: 43, a VH CDR2 comprising the amino acid sequence of SEQ ID NO: 45, a VH CDR3 comprising the amino acid sequence of SEQ ID NO: 47, a VL CDR1 comprising the amino acid sequence of SEQ ID NO: 49, a VL CDR2 comprising the amino acid sequence of SEQ ID NO: 51, and a VL CDR3 comprising the amino acid sequence of SEQ ID NO: 53. In one embodiment, the isolated antibody or fragment thereof that binds to PD-1 comprises a VH CDR1 comprising the amino acid sequence of SEQ ID NO: 55, a VH CDR2 comprising the amino acid sequence of SEQ ID NO: 57, a VH CDR3 comprising the amino acid sequence of SEQ ID NO: 59, a VL CDR1 comprising the amino acid sequence of SEQ ID NO: 61, a VL CDR2 comprising the amino acid sequence of SEQ ID NO: 63, and a VL CDR3 comprising the amino acid sequence of SEQ ID NO: 65. In one embodiment, the isolated antibody or fragment thereof that binds to PD-1 comprises a VH CDR1 comprising the amino acid sequence of SEQ ID NO: 67, a VH CDR2 comprising the amino acid sequence of SEQ ID NO: 69, a VH CDR3 comprising the amino acid sequence of SEQ ID NO: 71, a VL CDR1 comprising the amino acid sequence of SEQ ID NO: 73, a VL CDR2 comprising the amino acid sequence of SEQ ID NO: 75, and a VL CDR3 comprising the amino acid sequence of SEQ ID NO: 77. In one embodiment, the isolated antibody or fragment thereof that binds to PD-1 comprises a VH CDR1 comprising the amino acid sequence of SEQ ID NO: 17, a VH CDR2 comprising the amino acid sequence of SEQ ID NO: 19, a VH CDR3 comprising the amino acid sequence of SEQ ID NO: 21, a VL CDR1 comprising the amino acid sequence of SEQ ID NO: 24, a VL CDR2 comprising the amino acid sequence of SEQ ID NO: 80, and a VL CDR3 comprising the amino acid sequence of SEQ ID NO: 28 [e.g., the HL-7 mutant described herein]. In one embodiment, the isolated antibody or fragment thereof that binds to PD-1 comprises a VH CDR1 comprising the amino acid sequence of SEQ ID NO: 17, a VH CDR2 comprising the amino acid sequence of SEQ ID NO: 19, a VH CDR3 comprising the amino acid sequence of SEQ ID NO: 79, a VL CDR1 comprising the amino acid sequence of SEQ ID NO: 24, a VL CDR2 comprising the amino acid sequence of SEQ ID NO: 26, and a VL CDR3 comprising the amino acid sequence of SEQ ID NO: 28 [e.g., the HL-14 mutant described herein]. In one embodiment, the isolated antibody or fragment thereof that binds to PD-1 comprises a VH CDR1 comprising the amino acid sequence of SEQ ID NO: 78, a VH CDR2 comprising the amino acid sequence of SEQ ID NO: 19, a VH CDR3 comprising the amino acid sequence of SEQ ID NO: 21, a VL CDR1 comprising the amino acid sequence of SEQ ID NO: 24, a VL CDR2 comprising the amino acid sequence of SEQ ID NO: 26, and a VL CDR3 comprising the amino acid sequence of SEQ ID NO: 28 [e.g., the HLkin-1 mutant described herein]. In one embodiment, the isolated antibody or fragment thereof that binds to PD-1 comprises a VH CDR1 comprising the amino acid sequence of SEQ ID NO: 78, a VH CDR2 comprising the amino acid sequence of SEQ ID NO: 19, a VH CDR3 comprising the amino acid sequence of SEQ ID NO: 21, a VL CDR1 comprising the amino acid sequence of SEQ ID NO: 24, a VL CDR2 comprising the amino acid sequence of SEQ ID NO: 80, and a VL CDR3 comprising the amino acid sequence of SEQ ID NO: 28 [e.g., the HLkin-1 HL-7 mut2 mutant described herein]. In one embodiment, the isolated antibody or fragment thereof that binds to PD-1 comprises a VH CDR1 comprising the amino acid sequence of SEQ ID NO: 78, a VH CDR2 comprising the amino acid sequence of SEQ ID NO: 19, a VH CDR3 comprising the amino acid sequence of SEQ ID NO: 79, a VL CDR1 comprising the amino acid sequence of SEQ ID NO: 24, a VL CDR2 comprising the amino acid sequence of SEQ ID NO: 80, and a VL CDR3 comprising the amino acid sequence of SEQ ID NO: 28 [e.g., the HLkin-1 HL-7 HL-14 mut3 mutant described herein].

An aspect of the invention is directed to an isolated antibody or fragment thereof that binds to human Programmed cell death 1 (PD-1) protein. In one embodiment, the isolated antibody or fragment thereof that binds to human PD-1 protein comprises a heavy chain variable region comprising an amino acid sequence selected from the group consisting of SEQ ID NOS: 1, 3, 5, 7, 9, 12, 13, and 15, and a light chain variable region comprising an amino acid sequence selected from the group consisting of SEQ ID NOS: 2, 4, 6, 8, 10, and 11.

In other embodiments, the isolated antibody or fragment thereof that binds to human PD-1 protein comprises

In other embodiments, the isolated antibody or fragment thereof that binds to human PD-1 protein comprises. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 1, and the light chain comprises an amino acid sequence about 95% identical to SEQ ID NO: 2. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 3, and the light chain comprises an amino acid sequence about 95% identical to SEQ ID NO: 4. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 5, and the light chain comprises an amino acid sequence about 95% identical to SEQ ID NO: 6. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 7, and the light chain comprises an amino acid sequence about 95% identical to SEQ ID NO: 8. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 9, and the light chain comprises an amino acid sequence about 95% identical to SEQ ID NO: 10. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 1, and the light chain comprises an amino acid sequence about 95% identical to SEQ ID NO: 11. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 12, and the light chain comprises an amino acid sequence about 95% identical to SEQ ID NO: 2. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 13, and the light chain comprises an amino acid sequence about 95% identical to SEQ ID NO: 2. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 13, and the light chain comprises an amino acid sequence about 95% identical to SEQ ID NO: 11. In other embodiments, the isolated antibody or fragment thereof that binds to human PD-1 protein comprises a heavy chain, a light chain, or a combination thereof, wherein the heavy chain comprises an amino acid sequence about 95% identical to SEQ ID NO: 15, and the light chain comprises an amino acid sequence about 95% identical to SEQ ID NO: 11.

An aspect of the invention is directed to an isolated bispecific antibody that comprises a first antibody fragment that binds to human PD-1 protein and a second antigen-binding fragment having specificity to a molecule on an immune cell. In one embodiment, the isolated bispecific antibody comprises a fragment of a human antibody directed to the PD-1 protein as described herein. In some embodiments, the molecule on an immune cell comprises B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-1BB, TIGIT, VISTA, HEVM, BTLA, or KIR. In some embodiments, the antibody fragment that binds to human PD-1 protein comprises a Fab fragment, a single-chain variable fragment (scFv), or a single-domain antibody. In other embodiments, second antigen-binding fragment having specificity to a molecule on an immune cell comprises a Fab fragment, a single-chain variable fragment (scFv), or a single-domain antibody. In some embodiments, the bispecific antibody comprises an Fc fragment.

An aspect of the invention is directed to an isolated multispecific antibody that comprises a first antibody fragment that binds to human PD-1 protein as well as a second and a third antigen-binding fragment having specificity to a molecule on an immune cell. In one embodiment, the isolated multispecific antibody comprises a fragment of a human antibody directed to the PD-1 protein as described herein. In some embodiments, the molecule on an immune cell comprises B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-1BB, TIGIT, VISTA, HEVM, BTLA, or KIR. In some embodiments, the antibody fragment that binds to human PD-1 protein comprises a Fab fragment, a single-chain variable fragment (scFv), or a single-domain antibody. In other embodiments, the second and third antigen-binding fragment having specificity to a molecule on an immune cell comprises a Fab fragment, a single-chain variable fragment (scFv), or a single-domain antibody. In some embodiments, the multispecific antibody comprises an Fc fragment. In some embodiments, the multispecific antibody further comprises a fourth and/or fifth antigen-binding fragment having specificity to a molecule on an immune cell.

An aspect of the invention is directed to a nucleic acid encoding the isolated monoclonal antibody or antigen-binding fragment thereof that binds to human Programmed cell death 1 (PD-1) protein as described herein. An aspect of the invention is directed to a nucleic acid encoding the isolated antibody or fragment thereof that binds to human PD-1 protein as described herein. An aspect of the invention is directed to a nucleic acid encoding the bispecific antibody described herein. An aspect of the invention is directed to a nucleic acid encoding the multispecific antibody described herein. In some embodiments, the invention is directed to a vector comprising the nucleic acids described herein. In some embodiments, the invention is directed to cells comprising the vector described herein.

An aspect of the invention is directed to a pharmaceutical composition comprising the antibody or fragment that binds to human PD-1 protein as described herein, and a pharmaceutically acceptable carrier or excipient. In some embodiments, the pharmaceutical composition further comprises at least one additional therapeutic agent. In other embodiments, the therapeutic agent is a toxin, a radiolabel, a siRNA, a small molecule, or a cytokine.

An aspect of the invention is directed to a pharmaceutical composition comprising the bispecific antibody or fragment that binds to human PD-1 protein and a second antigen-binding fragment having specificity to a molecule on an immune cell as described herein, and a pharmaceutically acceptable carrier or excipient. In some embodiments, the pharmaceutical composition further comprises at least one additional therapeutic agent. In other embodiments, the therapeutic agent is a toxin, a radiolabel, a siRNA, a small molecule, or a cytokine.

An aspect of the invention is directed to a pharmaceutical composition comprising the bispecific antibody or fragment that binds to human PD-1 protein in addition to a second, third, fourth or fifth antigen-binding fragment having specificity to a molecule on an immune cell as described herein, and a pharmaceutically acceptable carrier or excipient. In some embodiments, the pharmaceutical composition further comprises at least one additional therapeutic agent. In other embodiments, the therapeutic agent is a toxin, a radiolabel, a siRNA, a small molecule, or a cytokine.

An aspect of the invention is directed to an isolated cell comprising one or more polynucleotide(s) encoding the PD-1 antibody or fragment described herein. An aspect of the invention is directed to an isolated cell comprising one or more polynucleotide(s) encoding the bispecific antibody or fragment thereof described herein. An aspect of the invention is directed to an isolated cell comprising one or more polynucleotide(s) encoding the multispecific antibody or fragment thereof described herein.

An aspect of the invention is directed to a kit comprising: the pharmaceutical compositions described herein; a syringe, needle, or applicator for administration of the pharmaceutical composition to a subject; and instructions for use.

An aspect of the invention is directed to an engineered cell comprising a chimeric antigen receptor, wherein the chimeric antigen receptor comprises an extracellular ligand binding domain that is specific for an antigen on the surface of a cancer cell, wherein the antigen comprises PD-1. Another aspect of the invention is directed to an engineered cell comprising a chimeric antigen receptor, wherein the chimeric antigen receptor comprises an extracellular ligand binding domain that is specific for a first antigen and a second antigen on the surface of a cancer cell, wherein the first antigen comprises PD-1 and the second antigen comprises a tumor specific surface antigen described herein. In one embodiment, the extracellular ligand binding domain comprises an antibody or fragment thereof. In another embodiment, the antibody comprises a VH and/or VL according to Tables 1-11, or any combination of heavy chains or light chains described herein. In one embodiment, the antibody comprises a CDR1, CDR2, and/or CDR3 of Table 12, or any combination of CDRs described herein. In one embodiment, the engineered cell comprises a T cell, an NK cell, or an NKT cell. In one embodiment, the T cell is CD4+, CD8+, CD3+ panT cells, or any combination thereof.

An aspect of the invention is directed to a method of treating cancer in a subject. In some embodiments, the method comprises administering to a subject in need thereof a therapeutically effective amount of a composition comprising a PD-1 antibody described herein. In some embodiments, the method comprises administering to a subject in need thereof a therapeutically effective amount of a composition comprising a pharmaceutical composition described herein. In some embodiments, the method comprises administering to a subject in need thereof a therapeutically effective amount of a composition comprising the CAR composition described herein. In one embodiment, the cancer expresses PD-1. In another embodiment, the cancer comprises non-small-cell lung cancer, melanoma, ovarian cancer, lymphoma, or renal-cell cancer. In some embodiments, the method further comprises administering to the subject a chemotherapeutic agent.

Other objects and advantages of this invention are readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a schematic of the PMPL panning strategy for antibody discovery (e.g., PD-1 antibodies of the invention).

FIG. 2 is a schematic of the VH and VL sequences for the anti-PD-1 antibody, P4-B3.

FIG. 3 is an illustration of 3D protein structure of human PD-1 with the differences between human and cyno PD-1 highlighted in red. The corresponding amino acid sequences are aligned below. High degree of similarity is observed between human and cyno monkey PD-1. A 3D protein structure of PD-1 binding with nivolumab is also shown.

FIG. 4 is a graph showing binding curves for P4-B3 minibodies against human and cyno PD-1.

FIG. 5 shows graphs of octet binding curves for different formats of P4-B3.

FIG. 6 shows binding curves of PD-L1 competition assays using PD-1 antibodies.

FIG. 7 shows binding curves of IgG ELISAs.

FIG. 8 shows FACS analysis plots for PD1 FACS conducted with anti-PD1 IgGs.

FIG. 9 is a schematic of a PD1-PDL1 bioassay.

FIG. 10 shows graphs of induction curves from a commercial PD1-PDL1 bioassay. (A) IgG1 wt monomer version of P4-B3 vs pembro and nivo. As can be seen, P4-B3 anti-PD1 antibody achieves ˜½ the signal of pembro and nivo. (B) Hexamer comparison for IgG1 LALA configurations. The hexamer configuration shows around a 2-3 fold shift in the dose response curve. (C) Direct comparison between IgG4 constructs (mono and hex) and nivo. Here similar trends are observed as in 10A and 10B. The commercial antibodies are 2× stronger than P4-B3 and the hexamer has ˜2-3 fold shift compared to the monomer.

FIG. 11 is a schematic of a ribbon diagram of human PD-1 (See Cheng, X et al., (2013). JBC doi.org/10.1074/jbc.M112.448126). PD-1 is an anti-parallel B-sandwich. An Anti-parallel B-sandwich is depicted. The front sheet of the PD-1 ribbon diagram comprises G, F, C, C′; the back sheet of the PD-1 ribbon diagram comprises A, B, E, D. PD-1 lacks cysteine in the stalk region, which prevents PD-1 from homodimerization.

FIG. 12 is a schematic of protein structure showing the interaction of PD-1 with its ligands, PDL-1 or PDL-2. See Cheng et al, Structure and Interactions of the Human Programmed Cell Death 1 Receptor, JBC 2013; Tan et al. (2016) Protein Cell DOI: 10.1007/s13238-016-0337-7; and Yan et al. (2008) PNAS, DPO: 10.1073/pnas.0804453105.

FIG. 13 shows ribbon diagrams of PD-1 binding to commercial antibodies. (A) Nivo blocks PD-L1 by binding to FG loop. (B) Pembro blocks by binding to C and C′ strands. See Fessas et al, Seminars in Oncology, 2017.

FIG. 14 is a protein model overlay and amino acid sequence comparison of human vs. mouse PD-1. The degree of similarity between human and mouse PD-1: ˜64%. See Cheng, X et al., (2013). JBC doi.org/10.1074/jbc.M112.448126.

FIG. 15 is a protein model overlay and amino acid sequence comparison of human vs. mouse PD-1. Amino acid residue P110 (purple) imposes a twist in the FG loop. In mouse PD1, this residue forces the BC loop towards the DE loop due to hydrophobic interactions between Arg83 and Trp39. Amino acid residue P63 (blue) in human PD-1 forces the loop away from C′ strand, creating highly flexible loop. Without wishing to be bound by theory, these two structural differences play a role in Pembro and Nivo's lack of cross reactivity with mouse PD-1. See Cheng, X et al., (2013). JBC doi.org/10.1074/jbc.M112.448126.

FIG. 16 is a graph showing P4-B3 binding to mouse PD-1. P4-B3 has reasonable affinity to mouse PD-1, setting it apart from Pembro and Nivo.

FIG. 17 is a schematic of staining strategies that can be used to differentially label the displayed yeast library prior to screening by FACS. See Cherf and Cochran, 2015, Methods Mol Biol.

FIG. 18 shows plots of FACS analyses. Standard staining sorting is shown where blue gates are positive hits, green gates are negative. The blue gates shift upwards along the x=y axis. Without wishing to be bound by theory, the PD-1 antibody clones bind PD-1 with higher affinity.

FIG. 19 shows plots of FACS analyses of kinetic staining. Collected cells in the blue gate, example of target is in the red circle. Collection gate was kept broad so as to obtain more samples.

FIG. 20 is a graph of a binding curve for P4-B3 mutants.

FIG. 21 is a graph of a binding curve for P4-B3 mutants.

FIG. 22 is a schematic of P4-B3 (anti-PD1) germline alignment and a diagram of the amino acid residues changed in the P4-B3 mutants generated.

FIG. 23 shows graphs of octet binding curves for different P4-B3 mutants. The SA sensor was coated with 2.5 ug/ml biotinylated PD-1.

FIG. 24 shows binding curves of PD-L1 competition assays using PD-1 antibodies (various P4-B3 mutants).

FIG. 25 is a schematic of the amino acid residues changed in the P4-B3 mutants generated.

FIG. 26 is a schematic of germline alignments of anti-PD1 antibody clones. These candidates were found via soluble protein panning (PD1-hFc).

FIG. 27 shows graphs of octet binding curves. Both PD1 and PDL1 are his tagged. As can be seen by sensor H4, the sensors were not saturated before adding the PDL1. Further sequencing confirmed that PD1 #5 was not an antibody. A4: R&D anti-PD1 (AF1086); B4: PD1 mini3; C4: PD1 mini4; D4: PD1 mini5; E4: PD1 mini7; F4: PD1 mini13; G4: TIG1 (control ab)+PDL1; H4: no Ab+PD1 to see if sensor is saturated.

FIG. 28 shows graphs of octet binding curves. Both PD1 and PDL1 are his tagged, as can be seen by sensor H4. The sensors were not saturated before adding the PDL1. PD1 and PDL1 were used at 2.5 ug/ml. Antibodies were used at 2 ug/ml. All samples diluted in 1×PBST. New PD-1 antibodies were used in scFv-Fc format, Nivo and Pembro are the commercial preparations. A6: Nivo; B6: Pembro; C6: PD1 #3; D6: PD1 #4; E6: PD1 #5; F6: PD1 #7; G6: PD1 #13; H6: TIG1 (−).

FIG. 29 shows graphs of octet binding curves. SA sensors were loaded with 2.5 ug expi293 expressed soluble PD1-avi and biotinylated via Avidity's biotinylation kit. PD1 #3 displayed a high off rate.

FIG. 30 is a schematic of germline alignments of anti-PD1 antibody clone, P4-B7.

FIG. 31 shows a graph of mini-body binding curves for P4-B7 to human and cyno PD-1. Curves were generated with expi293 cells 48 hours after transfection. Human variants were normalized to expression levels via commercial antibodies, however the cyno variants were not. Cyno variants were not normalized because the commercial antibodies used are not reported to bind to cyno PD #1.

FIG. 32 shows graphs of binding curves for an IgG ELISA using P4-B7. P4-B7 is shifted so far to the right, that the kinetics were not suitable to proceed. TOP, ELISA plates were coated with 1 ug/ml soluble PD1 for 2 hours at 37° C. The plates were then washed and blocked with 2% BSA/PBS at 37° C. for 1 hour. The blocking solution was removed and 3× serial dilutions of the antibodies were added to each well (100 ul) in 2% milk-PBST, starting with 6 ug/ml. The plates were then incubated at RT with gentle shaking, washed 6× with PBS-T, and the secondary anti-human Fc-HRP (1:150k, Bethyl) was added. The plates were again incubated at RT with gentle shaking for 1 hour before being washed 6× with PBS-T. TMB substrate was added and the plate was incubated at 30° C. for 10 min to accelerate the HRP reaction. The signal was then quenched with TMB stop solution and read at 450 nm. BOTTOM, The protocol for the data obtained was the same as the protocol for the data obtained in the TOP graph except that the plate was coated with 3× serial dilutions of the antigen, starting at 6 ug/ml. The antibody was then added at a constant concentration of 1 ug/ml to all wells.

FIG. 33 shows graphs of induction curves from a commercial PD1-PDL1 bioassay.

FIG. 34 shows schematic of Promega PD1-PDL1 bioassay (J1250). Promega PD1-PDL1 bioassay (J1250) was carried out with the wildtype aPD-1 scFv-Fc (P4-B3) and the mutants single and combo mutants generated from the random mutagenesis yeast library. Nivolumab was used as the benchmark control.

FIG. 35 shows P4-B3 mutant Promega bioassay (the scFv-Fc format bioassay). Nivo (black circles) reaches a fold induction of about 6, which is similar to our previous experiment. Single mutants HLkin-1, HL-7 and combo mutants Mut+2, Mut+3 demonstrate higher or equal levels of PD-1/PD-L1 blockade compared to Nivo. This is also reflected in the EC50 values, with Mut+2 having an EC50 value approximately half that of Nivo. P4-B3 wild type shows lower blockade levels and also has an EC50 value 1.75× greater than Nivo. The point mutations that were identified by our random mutagenesis yeast display library appear to have a significant effect on binding and checkpoint blockade ability. All P4-B3 samples used in this assay were in the scFv-Fc format. Only Nivo and F10 were used as full IgGs.

FIG. 36 shows octet binding curves for P4-B3 WT/mutant IgG. SA sensors were coated with biotinylated PD-1 and then dipped in varying concentrations of anti-PD1 antibody. The first step after the baseline shows association of the antibody, the second step shows disassociation. As can be seen in this figure, P4-B3 WT has a rapid off rate whereas the mutants and Pembro have a much slower off rate.

FIG. 37 shows binding curve for P4-B3 single versus combo mutants with mouse PD-1 (mPD-1); scFv-Fc-format.

FIG. 38 shows binding curve for P4-B3 single versus combo mutants with hPD1. scFv-Fc formats unless indicated.

FIG. 39 shows MFI for P4-B3 single versus combo mutants with hPD1. scFv-Fc formats except for pembro/nivo/WT IgG1.

FIG. 40 is a schematic of the amino acid residues changed in the P4-B3 mutants generated.

FIG. 41 is a schematic of the mixed lymphocyte reaction (MLR) assay. CD4+ T cells express high levels of PD-1 upon activation. DCs express high levels of PD-L1 to improve self tolerance in the body. T cell activation via MHC mismatch is limited due to PD-1/PD-L1 inhibition. Addition of anti-PD-1 antibodies remove this inhibitory signal leading to increased T cell activation (measured by cytokine release).

FIG. 42 shows graphs of the MLR assay depicting cytokine production, as indicated in the graph titles.

FIG. 43 shows graphs of the MLR assay depicting cytokine production, as indicated in the graph titles.

FIG. 44 shows statistical data tables of the MLR assay for Pembro vs P4B3 mut+3 IgG4.

FIG. 45 shows statistical data tables of the MLR assay for Pembro vs P4B3 mut+3 IgG4.

FIG. 46 shows statistical data tables of the MLR assay for Pembro vs P4B3 mut+3 IgG4.

FIG. 47 shows statistical data tables of the MLR assay for Pembro vs P4B3 mut+3 IgG4.

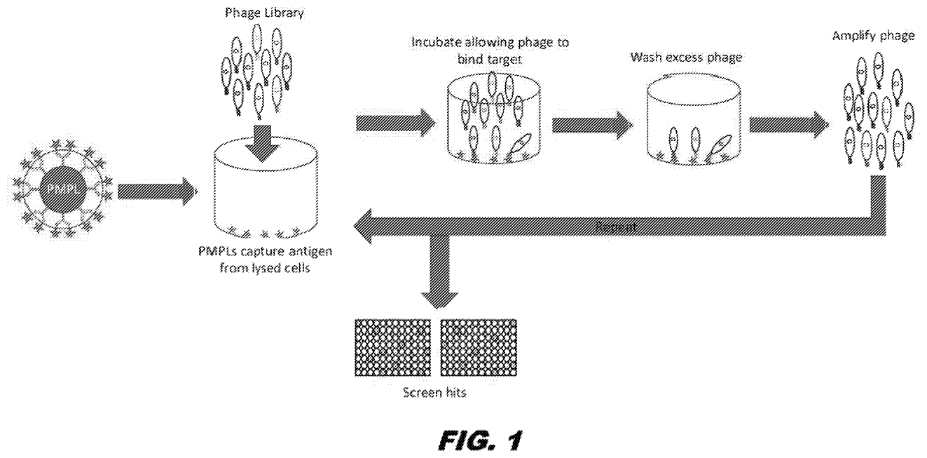

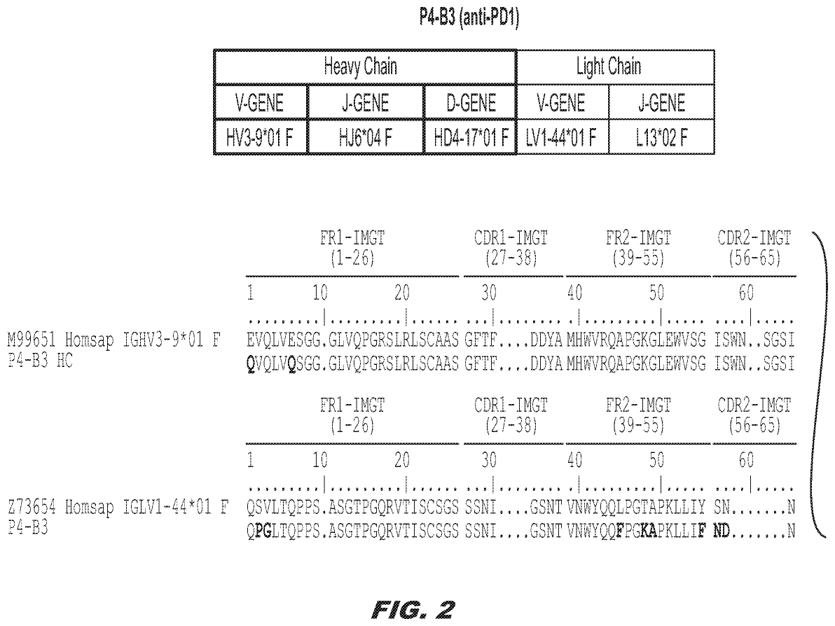

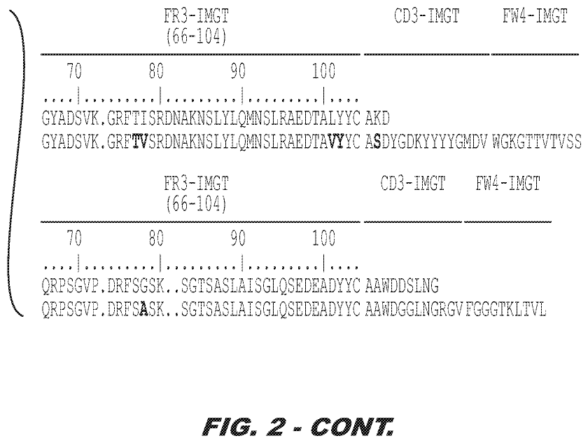

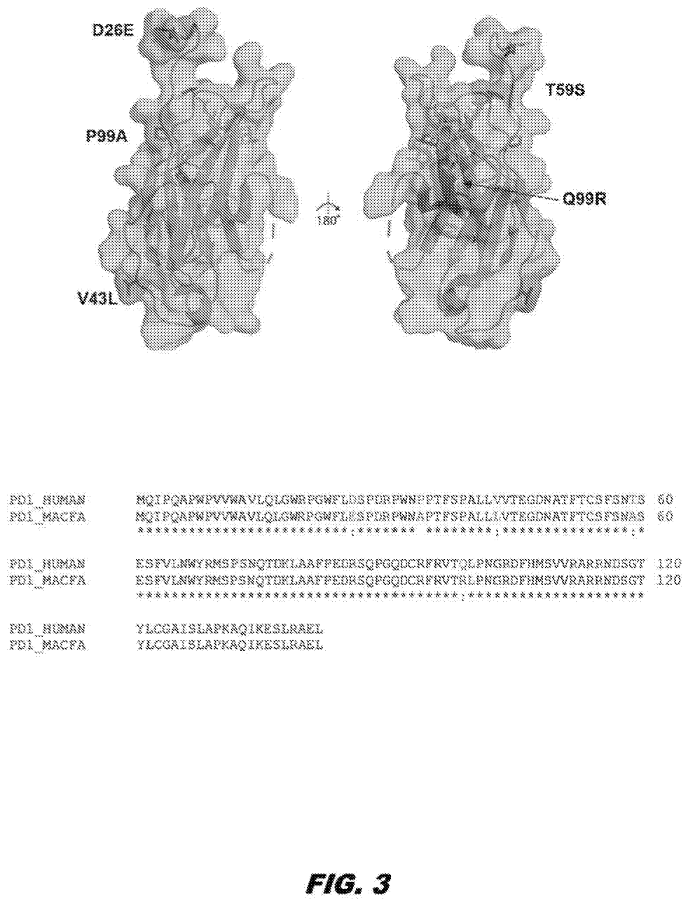

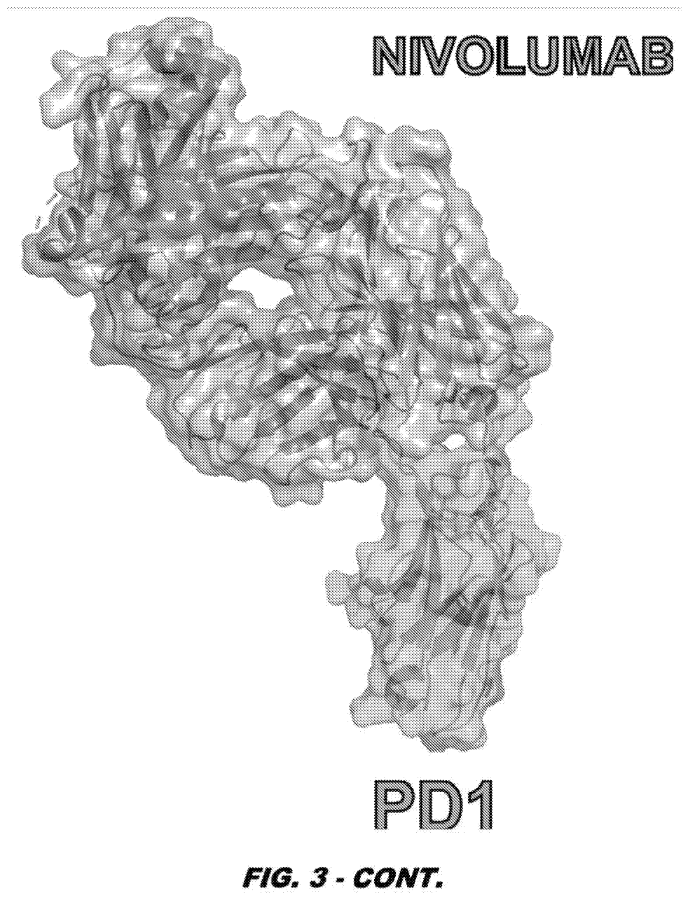

### DETAILED DESCRIPTION OF THE INVENTION

### Abbreviations and Definitions

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

PD-1

Programmed T cell death 1 (PD-1) is a trans-membrane protein found on the surface of T cells, which, when bound to programmed T cell death ligand 1 (PD-L1) on tumor cells, results in suppression of T cell activity and reduction of T cell-mediated cytotoxicity. Thus, PD-1 and PD-L1 are immune down-regulators or immune checkpoint “off switches”. Examples of PD-1 inhibitors include, but are not limited to, nivolumab, (Opdivo) (BMS-936558), pembrolizumab (Keytruda), pidilizumab, AMP-224, MEDI0680 (AMP-514), PDR001, MPDL3280A, MED14736, BMS-936559 and MSB0010718C.

The immune system must achieve a balance between effective responses to eliminate pathogenic entities and maintaining tolerance to prevent autoimmune disease. T cells are central to preserving this balance, and their proper regulation is primarily coordinated by the B7-CD28 family of molecules. Interactions between B7 family members, which function as ligands, and CD28 family members, which function as receptors, provide critical positive signals that not only initiate, augment and sustain T cell responses, but also contribute key negative signals that limit, terminate and/or attenuate T cell responses when appropriate. PD-1 is a member of the CD28 family.

Binding between PD-L1 and PD-1 has a profound effect on the regulation of T cell responses. Specifically, PD-L1/PD-1 interaction inhibits T cell proliferation and production of effector cytokines that mediate T cell activity and immune response, such as IL-2 and IFN-γ. This negative regulatory function is important for preventing T cell-mediated autoimmunity and immunopathology. However, the PD-1/PD-L1 axis has also been shown to play a role in T cell exhaustion, whereby the negative regulatory function inhibits T cell response to the detriment of the host. Prolonged or chronic antigenic stimulation of T cells can induce negative immunological feedback mechanisms which inhibit antigen-specific responses and results in immune evasion of pathogens. T cell exhaustion can also result in progressive physical deletion of the antigen-specific T cells themselves. T cell expression of PD-1 is up-regulated during chronic antigen stimulation, and its binding to PD-L1 results in a blockade of effector function in both CD4+(T helper cells) and CD8+(cytotoxic T lymphocytes or CTL) T cells, thus implicating the PD-1/PD-L1 interaction in the induction of T cell exhaustion.

More recently, studies showed that some chronic viral infections and cancers have developed immune evasion tactics that specifically exploit the PD-1/PD-L1 axis by causing PD-1/PD-L1-mediated T cell exhaustion. Many human tumor cells and tumor-associated antigen presenting cells express high levels of PD-L1, which suggests that the tumors induce T cell exhaustion to evade anti-tumor immune responses. During chronic HIV infection, for example, HIV-specific CD8+ T cells are functionally impaired, showing a reduced capacity to produce cytokines and effector molecules as well as a diminished ability to proliferate. Studies have shown that PD-1 is highly expressed on HIV-specific CD8+ T cells of HIV infected individuals, indicating that blocking the PD-1/PD-L1 pathway may have therapeutic potential for treatment of HIV infection and AIDS patients. Taken together, agents that block the PD-1/PD-L1 pathway will provide a new therapeutic approach for a variety of cancers, HIV infection, and/or other diseases and conditions that are associated with T-cell exhaustion. Therefore, there exists an urgent need for agents that can block or prevent PD-1/PD-L1 interaction.

PD-L1 overexpression has been detected in different cancers. For example, in breast cancer, PD-L1 is overexpressed and associated with high-risk prognostic factors. In renal cell carcinoma, PD-L1 is upregulated and increased expression of PD-1 has also been found in tumor infiltrating leukocytes. Anti-PD-L1 and anti-PD-1 antibodies have demonstrated some clinical efficacy in phase I trials for renal cell carcinoma. Therapeutic agents that can bind to PD-1 or PD-L1 may be useful for specifically targeting tumor cells. Agents that are capable of blocking the PD-1/PD-L1 interaction may be even more useful in treating cancers that have induced T cell exhaustion to evade anti-tumor T cell activity. Use of such agents, alone or in combination with other anti-cancer therapeutics, can effectively target tumor cells that overexpress PD-L1 and increase anti-tumor T cell activity, thereby augmenting the immune response to target tumor cells.

PD-1 and PD-L1 can also be upregulated by T cells after chronic antigen stimulation, for example, by chronic infections. During chronic HIV infection, HIV-specific CD8+ T cells are functionally impaired, showing a reduced capacity to produce cytokines and effector molecules as well as a diminished ability to proliferate. PD-1 is highly expressed on HIV-specific CD8+ T cells of HIV infected individuals. Therefore, blocking this pathway may enhance the ability of HIV-specific T cells to proliferate and produce cytokines in response to stimulation with HIV peptides, thereby augmenting the immune response against HIV. Other chronic infections may also benefit from the use of PD-1/PD-L1 blocking agents, such as chronic viral, bacterial or parasitic infections.

Aspects of the invention provide isolated monoclonal antibodies specific against PD-1. The term “isolated” as used herein with respect to cells, nucleic acids, such as DNA or RNA, refers to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides. The isolated antibodies were identified through the use of a 27 billion human single-chain antibody (scFv) phage display library via paramagnetic proteoliposomes, by using PD-1 as a library selection target. These antibodies represent a new class of monoclonal antibodies against PD-1 that can compete with PD-L1, pembrolizumab and nivolumab binding. Furthermore, the monoclonal PD-1 antibodies discussed herein cross react with cynomolgous monkey (Macaca fascicularis) PD-1 proteins. The monoclonal PD-1 antibodies discussed herein can also be used in the construction of multi-specific antibodies or as the payload for a CAR-T cell.

Ten unique recombinant monoclonal PD-1 antibodies are described herein. These include P4-B3, P4-B7, PD1 #2, PD1 #3, PD1 #13, P4-B3-HLkin-1, P4-B3-HL-7, P4-B3-HL-14, P4-B3 HLkin-1 HL-7 mut2, and P4-B3 HLkin-1 HL-7 HL-14 mut3. “Recombinant” as it pertains to polypeptides (such as antibodies) or polynucleotides refers to a form of the polypeptide or polynucleotide that does not exist naturally, a non-limiting example of which can be created by combining polynucleotides or polypeptides that would not normally occur together.

The nucleic acid and amino acid sequence of the monoclonal PD-1 antibodies are provided below, in addition to an exemplary wildtype IgG constant region useful in combination with the VH and VL sequences provided herein (see Table 2); the amino acid sequences of the heavy and light chain complementary determining regions CDRs of the PD-1 antibodies are underlined (CDR1), underlined and bolded (CDR2), or [symbol] [symbol] [symbol] below:

TABLE 1A
Ab P4-B3 Variable Region nucleic acid sequences

V_{H} chain of Ab P4-B3 VH (IGHV3-9*01)
CAGGTGCAGCTGGTGCAGTCTGGGGGAGGCTTGGTACAGCCTGGCAGG
TCCCTGAGACTCTCCTGTGCAGCCTCTGGATTCACCTTTGATGATTAT
GCCATGCACTGGGTCCGGCAAGCTCCAGGGAAGGGCCTGGAGTGGGTC
TCAGGTATTAGTTGGAATAGTGGTAGCATAGGCTATGCGGACTCTGTG
AAGGGCCGATTCACCGTCTCCAGAGACAACGCCAAGAACTCACTGTAT
CTGCAAATGAACAGCCTGAGAGCCGAGGACACGGCTGTGTATTACTGT
GCGAGTGACTACGGTGACAAATACTACTACTACGGTATGGACGTCTGG
GGCAAAGGGACCACGGTCACCGTCTCCTCA (SEQ ID NO: 94)
V_{L} chain of Ab P4-B3 VL (IGLV1-44*01)
CAGCCTGGGCTGACTCAGCCACCCTCAGCGTCTGGGACCCCCGGGCAG
AGGGTCACCATCTCTTGTTCTGGAAGCAGCTCCAACATCGGAAGTAAT
ACTGTCAACTGGTATCAGCAATTCCCCGGAAAGGCCCCCAAACTCCTC
ATCTTTAATGATAATCAGCGGCCCTCAGGGGTCCCTGACCGCTTCTCT
GCTTCCAAGTCTGGCACCTCAGCCTCCCTGGCCATTAGTGGCCTCCAG
TCTGAGGATGAGGCTGACTATTACTGTGCGGCATGGGATGGCGGTCTG
AATGGTCGAGGGGTGTTCGGCGGAGGGACCAAACTGACCGTCCTA
(SEQ ID NO: 95)

TABLE 1B
Ab P4-B3 Variable Region amino acid sequences

V_{H} chain of Ab P4-B3 VH (IGHV3-9*01)
QVQLVQSGGGLVQPGRSLRLSCAASGFTFDDYAMHWVRQAPGK
GLEWVSG [symbol] GYADSVKGRFTVSRDNAKNSLYLQMNSL
RAEDTAVYYC [symbol] WGKGTTVTVSS
(SEQ ID NO: 1)
V_{L} chain of Ab P4-B3 VL (IGLV1-44*01)
QPGLTQPPSASGTPGQRVTISCSGSSSNIGSNTVNWYQQFPGK
APKLLIF [symbol] QRPSGVPDRFSASKSGTSASLAISGLQSEDEAD
YYC [symbol] FGGGTKLTVL (SEQ ID NO: 2)

TABLE 2A
Ab P4-B3 Constant Region nucleic acid
sequences-wild type IgG1 monomer

CH1
ACCAAGGGCCCATCGGTCTTCCCCCTGGCACCCTCCTCCAAGAGCAC
CTCTGGGGGCACAGCGGCCCTGGGCTGCCTGGTCAAGGACTACTTCC
CCGAACCGGTGACGGTGTCGTGGAACTCAGGCGCCCTGACCAGCGGC
GTGCACACCTTCCCGGCTGTCCTACAGTCCTCAGGACTCTACTCCCT
CAGCAGCGTGGTGACCGTGCCCTCCAGCAGCTTGGGCACCCAGACCT
ACATCTGCAACGTGAATCACAAGCCCAGCAACACCAAGGTGGACAAG
AAA (SEQ ID NO: 116)
Hinge
GCAGAGCCCAAATCTTGTGACAAAACTCACACATGCCCACCGTGCCC
A (SEQ ID NO: 117)
CH2
GCACCTGAACTCCTGGGGGGACCGTCAGTCTTCCTCTTCCCCCCAAA
ACCCAAGGACACCCTCATGATCTCCCGGACCCCTGAGGTCACATGCG
TGGTGGTGGACGTGAGCCACGAAGACCCTGAGGTCAAGTTCAACTGG
TACGTGGACGGCGTGGAGGTGCATAATGCCAAGACAAAGCCGCGGGA
GGAGCAGTACAACAGCACGTACCGTGTGGTCAGCGTCCTCACCGTCC
TGCACCAGGACTGGCTGAATGGCAAGGAGTACAAGTGCAAGGTCTCC
AACAAAGCCCTCCCAGCCCCCATCGAGAAAACCATCTCCAAAGCCAA
A (SEQ ID NO: 118)
CH3
GGGCAGCCCCGAGAACCACAGGTGTACACCCTGCCCCCATCCCGGGA
TGAGCTGACCAAGAACCAGGTCAGCCTGACCTGCCTGGTCAAAGGCT
TCTATCCCAGCGACATCGCCGTGGAGTGGGAGAGCAATGGGCAGCCG
GAGAACAACTACAAGACCACGCCTCCCGTGCTGGACTCCGACGGCTC
CTTCTTCCTCTACAGCAAGCTCACCGTGGACAAGAGCAGGTGGCAGC
AGGGGAACGTCTTCTCATGCTCCGTGATGCATGAGGCTCTGCACAAC
CACTACACGCAGAAGAGCCTCTCCCTGTCTCCGGGTAAATGA
(SEQ ID NO: 119)
C_{L}
GGTCAGCCCAAGGCTGCCCCCTCGGTCACTCTGTTCCCGCCCTCCTC
TGAGGAGCTTCAAGCCAACAAGGCCACACTGGTGTGTCTCATAAGTG
ACTTCTACCCGGGAGCCGTGACAGTGGCCTGGAAGGCAGATGGCAGC
CCCGTCAAGGCGGGAGTGGAGACCACCACACCCTCCAAACAAAGCAA
CAACAAGTACGCGGCCAGCAGCTATCTGAGCCTGACGCCTGAGCAGT
GGAAGTCCCACAGAAGCTACAGCTGCCAGGTCACGCATGAAGGGAGC
ACCGTGGAGAAGACAGTGGCCCCTACAGAATGTTCATGA
(SEQ ID NO: 120)

TABLE 2B
Ab P4-B3 Constant Region amino acid
sequences-wild type IgG1 monomer

CH1
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKK (SEQ ID NO: 111)
Hinge
AEPKSCDKTHTCPPCP (SEQ ID NO: 112)
CH2
APELLGGPSVFLFPPKPKDTLMISRTPEVTCVVVDVSHEDPEVKFNW
YVDGVEVHNAKTKPREEQYNSTYRVVSVLTVLHQDWLNGKEYKCKVS
NKALPAPIEKTISKAK (SEQ ID NO: 113)
CH3
GQPREPQVYTLPPSRDELTKNQVSLTCLVKGFYPSDIAVEWESNGQP
ENNYKTTPPVLDSDGSFFLYSKLTVDKSRWQQGNVFSCSVMHEALHN
HYTQKSLSLSPGK (SEQ ID NO: 114)
C_{L}
GQPKAAPSVTLFPPSSEELQANKATLVCLISDFYPGAVTVAWKADGS
PVKAGVETTTPSKQSNNKYAASSYLSLTPEQWKSHRSYSCQVTHEGS
TVEKTVAPTECS (SEQ ID NO: 115)

TABLE 3A
Ab P4-B7 Variable Region nucleic acid sequences

V_{H} chain of Ab P4-B7 VH (IGHV5-51*01)
CAGGTGCAGCTGGTGCAGTCTGGAGCAGAGGTGAAGAAGCCCGGGGA
GTCTCTGAAGATCTCCTGTAAGGATTCTGGATACACCTTTACCACCT
ACTGGATCGGCTGGGTGCGCCAGCTGCCCGGGAAAGGCCTGGAGTTG
ATGGGGATCATCTATCCTGATGACTCTGATACCACATACAGCCCGTC
CTTCCAAGGCCATGTCACCATCTCAGCCGACAAGTCCATCAACACCG
CCTACCTGCAGTGGAGCAGCCTGAAGGCCTCGGACACCGCCATGTAT
TACTGTGCGTTTTGGGGTGCGAGTGGAGCGCCAGTGAATGGTTTTGA
TATCTGGGGCCAAGGCACCCTGGTCACCGTCTCCTCA
(SEQ ID NO: 96)
V_{L} chain of Ab P4-B7 VL (IGLV1-44*01)
CTGCCTGTGCTGACTCAGCCACCCTCAGCGTCTGGGACCCCCGGGCA
GAGGGTCACCATCTCCTGCACTGGGAGCAGCTCCAACATCGGGGCAG
GTTATGTTGTACACTGGTACCAGCAGCTCCCAGGAACGGCCCCCAAA
CTCCTCATCTATAGTAATAATCAGCGGCCCTCAGGGGTCCCTGACCG
ATTCTCTGGCTCCAAGTCTGGCACCTCAGCCTCCCTGGCCATCAGTG
GGCTCCAGTCTGAGGATGAGGCTGATTATTACTGTGCAGCATGGGAT
GACAGCCTGAATGCTCCGGTGTTCGGCGGAGGGACCAAGCTGACCGT
CCTA (SEQ ID NO: 97)

TABLE 3B
Ab P4-B7 Variable Region amino acid sequences

V_{H} chain of Ab P4-B7 VH (IGHV5-51*01)
QVQLVQSGAEVKKPGESLKISCKDSGYTFTTYWIGWVRQLPGKGLEL
MGI [symbol] TYSPSFQGHVTISADKSINTAYLQWSSLKASDTAMY
YC [symbol] WGQGTLVTVSS
(SEQ ID NO: 3)
V_{L} chain of Ab P4-B7 VL (IGLV1-44*01)
LPVLTQPPSASGTPGQRVTISCTGSSSNIGAGYVVHWYQQLPGTAPK
LLIY [symbol] QRPSGVPDRFSGSKSGTSASLAISGLQSEDEADYYC [symbol]
FGGGTKLTVLL (SEQ ID NO: 4)

TABLE 4A
PD1#2 Variable Region nucleic acid sequences

V_{H} chain of Ab PD1#2 VH (IGHV4-61*01)
CAGGTACAGCTGCAGCAGTCAGGCCCAGGACTGGTGAGGCCTTCGGCGA
CCCTGTCCCTCACCTGCACTGTCTCTGGTGACTCCGTCAGCAGTGATAA
TTACTTCTGGAGTTGGATTCGGCAGCCCCCAGGGAAGCCACTGGAGTGG
ATTGGCTATGTCTATTACAATGGGAACACCAACTACAACCCCTCCTTCA
ACAGTCGAGTCACCATGTCACTTGACACGTCCAAGAACCAGTTCTCCTT
GAAGCTGAGGTCTGTGACCGCCGCGGACACGGCCTTTTATTACTGTGCG
ACAGAGACGCCCCCAACCAGCTATTTTAATAGTGGACCCTTTGACTCCT
GGGGCCAGGGCACCCTGGTCACCGTCTCCTCG
(SEQ ID NO: 98)
V_{L} chain of Ab PD1#2 VL (IGLV10-54*01)
CAGCCTGGGCTGACTCAGCCACCCTCGGTGTCCAAGGGCTTGAGACAGA
CCGCCACACTCACCTGCACTGGGAGCAGCAACAATGTAGGCGCCCACGG
AGCAGCTTGGCTGCAGCAGCACCAGGGCCACCCTCCCAAACTCCTTGCC
TACAGGAATAACAACCGGCCCTCAGGGATCTCAGAGAGATTCTCTGCAT
CCAGGTCAGGAAACACAGCCTCCCTGACCATTATTGGACTCCAGCCTGA
GGACGAGGGTGACTATTACTGCTCATCATGGGACAGCAGCCTCAGTGGT
TATGTCTTCGGACCTGGGACCAAAGTCACCGTCCTA
(SEQ ID NO: 99)

TABLE 4B
Ab PD1#2 Variable Region amino acid sequences

V_{H} chain of Ab PD1#2 VH (IGHV4-61*01)
QVQLQQSGPGLVRPSATLSLTCTVSGDSVSSDNYFWSWIRQPPGKPLE
WIGY [symbol] NYNPSFNSRVTMSLDTSKNQFSLKLRSVTAADTAFYY
C [symbol] WGQGTLVTVSS
(SEQ ID NO: 5)
V_{L} chain of Ab PD1#2 VL (IGLV10-54*01)
QPGLTQPPSVSKGLRQTATLTCTGSSNNVGAHGAAWLQQHQGHPPKLL
AY [symbol] NRPSGISERFSASRSGNTASLTIIGLQPEDEGDYYC [symbol]
FGPGTKVTVL (SEQ ID NO: 6)

TABLE 5A
PD1#3 Variable Region nucleic acid sequences

V_{H} chain of Ab PD1#3 VH (IGHV1-18*01)
CAGGTGCAGCTGGTGCAGTCTGGGGCTGAGGTGAAGAAGCCTGGGTC
CTCAGTGAAGGTCTCCTGCAAGACTTCTGGCTACACCTTTAACAGGT
TTGGTCTCACCTGGGTGCGACAGGCCCCTGGACAAGGGCTTGAGTGG
ATGGGATGGACCAACCCTTACAATGGTAACACAAGGTATGCACAGAA
GTTCCAGGGCAGAGTCACCATGACCACAGACACATCCACGAGCACAG
CCTACATGGAGCTGAGGAGCCTGAGATCTGACGACACGGCCATGTAT
TTCTGTGCGAGAGTCGTAGCCGTAAACGGTATGGACGTCTGGGGCCA
AGGGACCACGGTCACCGTCTCCTCA (SEQ ID NO: 100)
V_{L} chain of Ab PD1#3 VL (IGLV6-57*01)
AATTTTATGCTGACTCAGCCCCACTCTGTGTCGGAGTCTCCGGGGAA
GACGGTTACCATCTCCTGCACCCGCAACAGTGGCAGCATTGCCGCCT
ACTATGTGCAGTGGTACCAGCAGCGCCCGGGCAGTTCCCCCACCACT
GTGATCTATGAAGATAACCAAAGACCCTCTGGGGTCCCTGATCGGTT
CTCTGGCTCCATCGACAGCTCCTCCAACTCTGCCTCCCTCACCATCT
CTGGACTGAAGACTGAGGACGAGGCTGACTACTACTGTCAGTCTTAT
GATAGCAGCAATCTTTGGGTGTTCGGCGGAGGGACCAAGCTGACCGT
CCTA (SEQ ID NO: 101)

TABLE 5B
Ab PD1#3 Variable Region amino acid sequences

V_{H} chain of Ab PD1#3 VH (IGHV1-18*01)
QVQLVQSGAEVKKPGSSVKVSCKTSGYTFNRFGLTWVRQAPGQGLEW
MGW [symbol] RYAQKFQGRVTMTTDTSTSTAYMELRSLRSDDTAMY
FC [symbol] WGQGTTVTVSS (SEQ ID NO: 7)
V_{L} chain of Ab PD1#3 VL (IGLV6-57*01)
NFMLTQPHSVSESPGKTVTISCTRNSGSIAAYYVQWYQQRPGSSPTT
VIY [symbol] QRPSGVPDRFSGSIDSSSNSASLTISGLKTEDEADYYC [symbol]
FGGGTKLTVL (SEQ ID NO: 8)

TABLE 6A
Ab PD1#13 Variable Region nucleic acid sequences

V_{H} chain of Ab PD1#13 VH (IGHV3-30*01)
GAGGTGCAGCTGGTGCAGTCTGGGGGAGGCGTGGTCCAGCCTGGGAGGTCCCTGAGAC
TCTCCTGTGCAGCCTCTGGATTCACCTTCAGTAGCTATGCTATGCACTGGGTCCGCCAGG
CTCCAGGCAAGGGGCTGGAGTGGGTGGCAGTTATATCATATGATGGAAGCAATAAATA
CTACGCAGACTCCGTGAAGGGCCGATTCACCATCTCCAGAGACAATTCCAAGAACACG
CTGTATCTGCAAATGAACAGCCTGAGAGCTGAGGACACGGCTGTGTATTACTGTGCGAG
CCAAACAGTGGCTGGAAGTGACTACTGGGGCCAGGGCACCCTGGTCACCGTCTCCTCA
(SEQ ID NO: 102)
V_{L} chain of Ab PD1#13 VL (IGLV1-44*01)
CAGCCTGGGCTGACTCAGCCACCCTCGGTGCCAGTGGCCCCAGGACAGACGGCCAGGA
TTACCTGTGGGGGAAACAACATTGGAAGTAAAAGTGTGCACTGGTACCAGCAGAAGCC
AGGCCAGGCCCCTGTGCTGGTCGTCTATGATGATAGCGACCGGCCCTCAGGGATCCCTG
AGCGATTCTCTGGCTCCAACTCTGGGAACACGGCCACCCTGACCATCAGCAGGGTCGAA
GCCGGGGATGAGGCCGACTATTACTGTCAGGTGTGGCATAGTGTTAGTGATCAAGGGG
TCTTCGGAACTGGGACCAAAGTCACCGTCCTA
(SEQ ID NO: 103)

TABLE 6B
Ab PD1#13 Variable Region amino acid sequences

V_{H} chain of Ab PD1#13 VH (IGHV3-30*01)
EVQLVQSGGGVVQPGRSLRLSCAASGFTFSSYAMHWVRQAPGKGLEWVAV [symbol] Y
YADSVKGRFTISRDNSKNTLYLQMNSLRAEDTAVYYC [symbol] WGQGTLVTVSS
(SEQ ID NO: 9)
V_{L} chain of Ab PD1#13 VL (IGLV1-44*01)
QPGLTQPPSVPVAPGQTARITCGGNNIGSKSVHWYQQKPGQAPVLVVY [symbol] DRPSGIPERFS
GSNSGNTATLTISRVEAGDEADYYC [symbol] FGTGTKVTVL
(SEQ ID NO: 10)

P4-B3 error prone mutants (mutations from P4-B3 highlighted in red, silent mutations in blue)

TABLE 7A
Ab P4-B3-HLkin1 Variable Region nucleic acid sequences

V_{H} chain of Ab HLkin1 VH
CAGGTGCAGCTGGTGCAGTCTGGGGGAGGCTTGGTACAGCCTGGCAGGTCCCTGAGAC
TCTCCTGTGCAGCCTCTGGATTCACCTTTGATGATT [symbol] TGCCATGCACTGGGTCCGGCAAG
CTCCAGGGAAGGGCCTGGAGTGGGTCTCAGGTATTAGTTGGAATAGTGGTAGCATAGG
CTATGCGGACTCTGTGAAGGGCCGATTCACCGTCTCCAGAGACAACGCCAAGAACTCA
CTGTATCTGCAAATGAACAGCCTGAGAGCCGAGGACACGGCTGTGTATTACTGTGCGA
GTGACTACGGTGACAAATACTACTACTACGGTATGGACGTCTGGGGCAAAGGGACCAC
GGTCACCGTCTCCTCA
(SEQ ID NO: 104)
V_{L} chain of Ab HLkin1 VL
CAGCCTGGGCTGACTCAGCCACCCTCAGCGTCTGGGACCCCCGGGCAGAGGGTCACCA
TCTCTTGTTCTGGAAGCAGCTCCAACATCGGAAGTAATACTGTCAACTGGTATCAGCAA
TTCCCCGGAAAGGCCCCCAAACTCCTCATCTTTAATGATAATCAGCGGCCCTCAGGGGT
CCCTGACCGCTTCTCTGCTTCCAAGTCTGGCACCTCAGCCTCCCTGGCCATTAGTGGCCT
CCAGTCTGAGGATGAGGCTGACTATTACTGTGCGGCATGGGATGGCGGTCTGAATGGTC
GAGGGGTGTTCGGCGGAGGGACCAAACTGACCGTCCTA
(SEQ ID NO: 105)

TABLE 7B
Ab HLkin1 Variable Region amino acid sequences

V_{H} chain of Ab HLKin1 VH
QVQLVQSGGGLVQPGRSLRLSCAASGFTFDD [symbol] AMHWVRQAPGKGLEWVSG [symbol] G
YADSVKGRFTVSRDNAKNSLYLQMNSLRAEDTAVYYC [symbol] WGKGTTV
TVSS
(SEQ ID NO: 13)
V_{L} chain of Ab HLKin1
QPGLTQPPSASGTPGQRVTISCSGSSSNIGSNTVNWYQQFPGKAPKLLIF [symbol] QRPSGVPDRF
SASKSGTSASLAISGLQSEDEADYYC [symbol] FGGGTKLTVL
(SEQ ID NO: 2)

TABLE 8A
Ab P4-B3-HL-7 Variable Region nucleic acid sequences

V_{H} chain of Ab HL-7 VH
CAGGTGCAGCTGGTGCAGTCTGGGGGAGGCTTGGTACAGCCTGGCAGGTCCCTGAGAC
TCTCCTGTGCAGCCTCTGGATTCACCTTTGATGATTATGCCATGCACTGGGTCCGGCAAG
CTCCAGGGAAGGGCCTGGAGTGGGTCTCAGGTATTAGTTGGAATAGTGGTAGCATAGG
CTATGCGGACTCTGTGAAGGGCCGATTCACCGTCTCCAGAGACAACGCCAAGAACTCA
CTGTATCTGCAAATGAACAGCCTGAGAGCCGAGGACACGGCTGTGTATTACTGTGCGA
GTGACTACGGTGACAAATACTACTACTACGGTATGGACGTCTGGGGCAAAGGGACCAC
GGTCACCGTCTCCTCA
(SEQ ID NO: 106)
V_{L} chain of Ab HL-7 VL
CAGCCTGGGCTGACTCAGCCACCCTCAGCGTCTGGGACCCC [symbol] GGGCAGAGGGTCACCA
TCTCTTGTTCTGGAAGCAGCTCCAACATCGGAAGTAATACTGTCAACTGGTATCAGCAA
TTCCCCGGAAAGGCCCCCAAACTCCTCATCTTT [symbol] ATGATAATCAGCGGCCCTCAGGGGT
CCCTGACCGCTTCTCTGCTTCCAAGTCTGGCACCTCAGCCTCCCTGGCCATTAGTGGCCT
CCAGTCTGAGGATGAGGCTGACTATTACTGTGCGGCATGGGATGGCGGTCTGAATGGTC
GAGGGGTGTTCGGCGGAGGGACCAAACTGACCGTCCTA
(SEQ ID NO: 107)

TABLE 8B
Ab HL-7 Variable Region amino acid sequences

V_{H} chain of Ab HL-7 VH
QVQLVQSGGGLVQPGRSLRLSCAASGFTFDDYAMHWVRQAPGKGLEWVSG [symbol] G
YADSVKGRFTVSRDNAKNSLYLQMNSLRAEDTAVYYC [symbol] WGKGTTV
TVSS
(SEQ ID NO: 1)
V_{L} chain of Ab HL-7
QPGLTQPPSASGTPGQRVTISCSGSSSNIGSNTVNWYQQFPGKAPKLLIF [symbol] QRPSGVPDRF
SASKSGTSASLAISGLQSEDEADYYC [symbol] FGGGTKLTVL
(SEQ ID NO: 11)

TABLE 9A
Ab P4-B3-HL-14 Variable Region nucleic acid sequences

V_{H} chain of Ab HL-14 VH
CAGGTGCAGCTGGTGCAGTCTGGGGGAGGCTTGGTACAGCCTGGCAGGTCCCTGAGAC
TCTCCTGTGCAGCCTCTGGATTCACCTTTGATGATTATGCCATGCACTGGGTCCGGCAAG
CTCCAGGGAAGGGCCTGGAGTGGGTCTCAGGTATTAGTTGGAATAGTGGTAGCATAGG
CTATGCGGACTCTGTGAAGGGCCGATTCACCGTCTCCAGAGACAACGCCAAGAACTCA
CTGTATCTGCAAATGAACAGCCTGAGAGCCGAGGACACGGCTGTGTATTACTGTGCGA
GTGACTACGGTGACAAATACT [symbol] CTACTACGGTATGGACGTCTGGGGCAAAGGGACCAC
GGTCACCGTCTCCTCA
(SEQ ID NO: 108)
V_{L} chain of Ab HL-14 VL
CAGCCTGGGCTGACTCAGCCACCCTCAGCGTCTGGGACCCCCGGGCAGAGGGTCACCA
TCTCTTGTTCTGGAAGCAGCTCCAACATCGGAAGTAATACTGTCAACTGGTATCAGCAA
TTCCCCGGAAAGGCCCCCAAACTCCTCATCTTTAATGATAATCAGCGGCCCTCAGGGGT
CCCTGACCGCTTCTCTGCTTCCAAGTCTGGCACCTCAGCCTCCCTGGCCATTAGTGGCCT
CCAGTCTGAGGATGAGGCTGACTATTACTGTGCGGCATGGGATGGCGGTCTGAATGGTC
GAGGGGTGTTCGGCGGAGGGACCAAACTGACCGTCCTA
(SEQ ID NO: 109)

TABLE 9B
Ab HL-14 Variable Region amino acid sequences

V_{H} chain of Ab HL-14 VH
QVQLVQSGGGLVQPGRSLRLSCAASGFTFDDYAMHWVRQAPGKGLEWVSG [symbol] G
YADSVKGRFTVSRDNAKNSLYLQMNSLRAEDTAVYYC [symbol] WGKGTTV
TVSS
(SEQ ID NO: 12)
V_{L} chain of Ab HL-14
QPGLTQPPSASGTPGQRVTISCSGSSSNIGSNTVNWYQQFPGKAPKLLIF [symbol] QRPSGVPDRF
SASKSGTSASLAISGLQSEDEADYYC [symbol] FGGGTKLTVL
(SEQ ID NO: 2)

TABLE 10A
Ab HLkin-1 HL-7 mut2 Variable Region nucleic acid sequences

V_{H} chain of Ab HLkin-1 HL-7 mut2 VH
CAGGTGCAGCTGGTGCAGTCTGGGGGAGGCTTGGTACAGCCTGGCAGGTCCCTGAGAC
TCTCCTGTGCAGCCTCTGGATTCACCTTTGATGATT [symbol] TGCCATGCACTGGGTCCGGCAAG
CTCCAGGGAAGGGCCTGGAGTGGGTCTCAGGTATTAGTTGGAATAGTGGTAGCATAGG
CTATGCGGACTCTGTGAAGGGCCGATTCACCGTCTCCAGAGACAACGCCAAGAACTCA
CTGTATCTGCAAATGAACAGCCTGAGAGCCGAGGACACGGCTGTGTATTACTGTGCGA
GTGACTACGGTGACAAATACTACTACTACGGTATGGACGTCTGGGGCAAAGGGACCAC
GGTCACCGTCTCCTCA
(SEQ ID NO: 104)
V_{L} chain of Ab HLkin-1 HL-7 mut2 VL
CAGCCTGGGCTGACTCAGCCACCCTCAGCGTCTGGGACCCCCGGGCAGAGGGTCACCA
TCTCTTGTTCTGGAAGCAGCTCCAACATCGGAAGTAATACTGTCAACTGGTATCAGCAA
TTCCCCGGAAAGGCCCCCAAACTCCTCATCTTT [symbol] ATGATAATCAGCGGCCCTCAGGGGT
CCCTGACCGCTTCTCTGCTTCCAAGTCTGGCACCTCAGCCTCCCTGGCCATTAGTGGCCT
CCAGTCTGAGGATGAGGCTGACTATTACTGTGCGGCATGGGATGGCGGTCTGAATGGTC
GAGGGGTGTTCGGCGGAGGGACCAAACTGACCGTCCTA
(SEQ ID NO: 107)

TABLE 10B
Ab HLkin-1 HL-7 mut2 Variable Region amino acid sequences

V_{H} chain of Ab HLkin-1 HL-7 mut2 VH
QVQLVQSGGGLVQPGRSLRLSCAASGFTFDD [symbol] AMHWVRQAPGKGLEWVSG [symbol] G
YADSVKGRFTVSRDNAKNSLYLQMNSLRAEDTAVYYC [symbol] WGKGTTV
TVSS
(SEQ ID NO: 13)
V_{L} chain of Ab HLkin-1 HL-7 mut2
QPGLTQPPSASGTPGQRVTISCSGSSSNIGSNTVNWYQQFPGKAPKLLIF [symbol] QRPSGVPDRF
SASKSGTSASLAISGLQSEDEADYYC [symbol] FGGGTKLTVL
(SEQ ID NO: 11)

TABLE 11A
Ab HLkin-1 HL-7 HL-14 mut3 Variable Region
nucleic acid sequences

V_{H} chain of Ab HLkin-1 HL-7 HL-14 mut3 VH
CAGGTGCAGCTGGTGCAGTCTGGGGGAGGCTTGGTACAGCCTGGCAGGTCCCTGAGAC
TCTCCTGTGCAGCCTCTGGATTCACCTTTGATGATT [symbol] CTGCCATGCACTGGGTCCGGCAAG
CTCCAGGGAAGGGCCTGGAGTGGGTCTCAGGTATTAGTTGGAATAGTGGTAGCATAGG
CTATGCGGACTCTGTGAAGGGCCGATTCACCGTCTCCAGAGACAACGCCAAGAACTCA
CTGTATCTGCAAATGAACAGCCTGAGAGCCGAGGACACGGCTGTGTATTACTGTGCGA
GTGACTACGGTGACAAATACT [symbol] CTACTACGGTATGGACGTCTGGGGCAAAGGGACCAC
GGTCACCGTCTCCTCA
(SEQ ID NO: 110)
V_{L} chain of Ab HLkin-1 HL-7 HL-14 mut3 VL
CAGCCTGGGCTGACTCAGCCACCCTCAGCGTCTGGGACCCCCGGGCAGAGGGTCACCA
TCTCTTGTTCTGGAAGCAGCTCCAACATCGGAAGTAATACTGTCAACTGGTATCAGCAA
TTCCCCGGAAAGGCCCCCAAACTCCTCATCTTT [symbol] ATGATAATCAGCGGCCCTCAGGGGT
CCCTGACCGCTTCTCTGCTTCCAAGTCTGGCACCTCAGCCTCCCTGGCCATTAGTGGCCT
CCAGTCTGAGGATGAGGCTGACTATTACTGTGCGGCATGGGATGGCGGTCTGAATGGTC
GAGGGGTGTTCGGCGGAGGGACCAAACTGACCGTCCTA
(SEQ ID NO: 107)

TABLE 11B
Ab HLkin-1 HL-7 HL-14 mut3 Variable Region amino acid sequences

V_{H} chain of Ab HLkin-1 HL-7 HL-14 mut3
QVQLVQSGGGLVQPGRSLRLSCAASGFTFDD [symbol] AMHWVRQAPGKGLEWVSG [symbol] G
YADSVKGRFTVSRDNAKNSLYLQMNSLRAEDTAVYYC [symbol] WGKGTTV
TVSS
(SEQ ID NO: 15)
V_{L} chain of Ab HLkin-1 HL-7 HL-14 mut3
QPGLTQPPSASGTPGQRVTISCSGSSSNIGSNTVNWYQQFPGKAPKLLIF [symbol] QRPSGVPDRF
SASKSGTSASLAISGLQSEDEADYYC [symbol] FGGGTKLTVL
(SEQ ID NO: 11)

The amino acid sequences of the heavy and light chain complementary determining regions of the PD-1 antibodies are shown in Table 12A-B below:

TABLE 12A
Heavy chain (V_{H}) complementary determining regions
(CDRs) of the PD-1 antibodies

| Sequence | | | |
|---|---|---|---|
| ID | V_{H} CDR1 | V_{H} CDR2 | V_{H} CDR3 |
| P4-B3 | GFTFDDYA | ISWNSGSI | ASDYGDKYYYYGMDV |
| | (SEQ ID NO: 17) | (SEQ ID NO: 19) | (SEQ ID NO: 21) |
| P4-B7 | GYTFTTYW | IYPDDSDT | AFWGASGAPVNGFDI |
| | (SEQ ID NO: 31) | (SEQ ID NO: 33) | (SEQ ID NO: 35) |
| PD1#2 | GDSVSSDNYF | VYYNGNT | ATETPPTSYFNSGPFDS |
| | (SEQ ID NO: 43) | (SEQ ID NO: 45) | (SEQ ID NO: 47) |
| PD1#3 | GYTFNRFG | TNPYNGNT | ARVVAVNGMDV |
| | (SEQ ID NO: 55) | (SEQ ID NO: 57) | (SEQ ID NO: 59) |
| PD1#13 | GFTFSSYA | ISYDGSNK | ASQTVAGSDY |
| | (SEQ ID NO: 67) | (SEQ ID NO: 69) | (SEQ ID NO: 71) |
| HL-7 | GFTFDDYA | ISWNSGSI | ASDYGDKYYYYGMDV |
| | (SEQ ID NO: 17) | (SEQ ID NO: 19) | (SEQ ID NO: 21) |
| HL-14 | GFTFDDYA | ISWNSGSI | ASDYGDKY [symbol] YYGMDV |
| | (SEQ ID NO: 17) | (SEQ ID NO: 19) | (SEQ ID NO: 79) |
| HLkin-1 | GFTFDD [symbol] A | ISWNSGSI | ASDYGDKYYYYGMDV |
| | (SEQ ID NO: 78) | (SEQ ID NO: 19) | (SEQ ID NO: 21) |
| HLkin-1 | GFTFDD [symbol] A | ISWNSGSI | ASDYGDKYYYYGMDV |
| HL-7 mut2 | (SEQ ID NO: 78) | (SEQ ID NO: 19) | (SEQ ID NO: 21) |
| HLkin-1 | GFTFDD [symbol] A | ISWNSGSI | ASDYGDKY [symbol] NYGMDV |
| HL-7 HL-14 | (SEQ ID NO: 78) | (SEQ ID NO: 19) | (SEQ ID NO: 79) |
| mut3 | | | |

TABLE 12B
Light chain (V_{L}) complementary determining
regions (CDRs) of the PD-1 antibodies

| Sequence | | | |
|---|---|---|---|
| ID | V_{L} CDR1 | V_{L} CDR2 | V_{L} CDR3 |
| P4-B3 | SSNIGSNT | NDN | AAWDGGLNGRGV |
| | (SEQ ID NO: 24) | (SEQ ID NO: 26) | (SEQ ID NO: 28) |
| P4-B7 | SSNIGAGYV | SNN | AAWDDSLNAPV |
| | (SEQ ID NO: 37) | (SEQ ID NO: 39) | (SEQ ID NO: 41) |
| PD1#2 | SNNVGAHG | RNN | SSWDSSLSGYV |
| | (SEQ ID NO: 49) | (SEQ ID NO: 51) | (SEQ ID NO: 53) |
| PD1#3 | SGSIAAYY | EDN | QSYDSSNLWV |
| | (SEQ ID NO: 61) | (SEQ ID NO: 63) | (SEQ ID NO: 65) |
| PD1#13 | NIGSKS | DDS | QVWHSVSDQGV |
| | (SEQ ID NO: 73) | (SEQ ID NO: 75) | (SEQ ID NO: 77) |
| HL-7 | SSNIGSNT | [symbol] DN | AAWDGGLNGRGV |
| | (SEQ ID NO: 24) | (SEQ ID NO: 80) | (SEQ ID NO: 28) |
| HL-14 | SSNIGSNT | NDN | AAWDGGLNGRGV |
| | (SEQ ID NO: 24) | (SEQ ID NO: 26) | (SEQ ID NO: 28) |
| HLkin-1 | SSNIGSNT | NDN | AAWDGGLNGRGV |
| | (SEQ ID NO: 24) | (SEQ ID NO: 26) | (SEQ ID NO: 28) |
| HLkin-1 | SSNIGSNT | [symbol] DN | AAWDGGLNGRGV |
| HL-7 mut2 | (SEQ ID NO: 24) | (SEQ ID NO: 80) | (SEQ ID NO: 28) |
| HLkin-1 | SSNIGSNT | [symbol] DN | AAWDGGLNGRGV |
| HL-7 HL-14 | (SEQ ID NO: 24) | (SEQ ID NO: 80) | (SEQ ID NO: 28) |
| mut3 | | | |

The amino acid sequences of the heavy and light chain framework regions of the PD-1 antibodies are shown in Table 13A-B below:

TABLE 13A
Heavy chain (V_{H}) framework regions (FRs) of the PD-1 antibodies

| Seq ID | VH FR1 | VH FR2 | VH FR3 | VH FR4 |
|---|---|---|---|---|
| P4-B3 | QVQLVQSGGGLVQ | MHWVRQAPGK | GYADSVKGRFTVSRDN | WGKGTTVTVSS |
| | PGRSLRLSCAAS | GLEWVSG | AKNSLYLQMNSLRAED | (SEQ ID NO: 22) |
| | (SEQ ID NO: 16) | (SEQ ID NO: 18) | TAVYYC | |
| | | | (SEQ ID NO: 20) | |
| P4-B7 | QVQLVQSGAEVKK | IGWVRQLPGKGL | TYSPSFQGHVTISADKS | WGQGTLVTVSS |
| | PGESLKISCKDS | ELMGI | INTAYLQWSSLKASDT | (SEQ ID NO: 22) |
| | (SEQ ID NO: 30) | (SEQ ID NO: 32) | AMYYC | |
| | | | (SEQ ID NO: 34) | |
| PD1#2 | QVQLQQSGPGLVR | WSWIRQPPGKPL | NYNPSFNSRVTMSLDT | WGQGTLVTVSS |
| | PSATLSLICTVS | EWIGY | SKNQFSLKLRSVTAAD | (SEQ ID NO: 22) |
| | (SEQ ID NO: 42) | (SEQ ID NO: 44) | TAFYYC | |
| | | | (SEQ ID NO: 46) | |
| PD1#3 | QVQLVQSGAEVKK | LTWVRQAPGQG | RYAQKFQGRVTMTTD | WGQGTTVTVSS |
| | PGSSVKVSCKTS | LEWMGW | TSTSTAYMELRSLRSD | (SEQ ID NO: 22) |
| | (SEQ ID NO: 54) | (SEQ ID NO: 56) | DTAMYFC | |
| | | | (SEQ ID NO: 58) | |
| PD1#13 | EVQLVQSGGGVVQ | MHWVRQAPGK | YYADSVKGRFTISRDNS | WGQGTLVTVSS |
| | PGRSLRLSCAAS | GLEWVAV | KNTLYLQMNSLRAEDT | (SEQ ID NO: 22) |
| | (SEQ ID NO: 66) | (SEQ ID NO: 68) | AVYYC | |
| | | | (SEQ ID NO: 70) | |
| HL-7 | QVQLVQSGGGLVQ | MHWVRQAPGK | GYADSVKGRFTVSRDN | WGKGTTVTVSS |
| | PGRSLRLSCAAS | GLEWVSG | AKNSLYLQMNSLRAED | (SEQ ID NO: 22) |
| | (SEQ ID NO: 16) | (SEQ ID NO: 18) | TAVYYC | |
| | | | (SEQ ID NO: 20) | |
| HL-14 | QVQLVQSGGGLVQ | MHWVRQAPGK | GYADSVKGRFTVSRDN | WGKGTTVTVSS |
| | PGRSLRLSCAAS | GLEWVSG | AKNSLYLQMNSLRAED | (SEQ ID NO: 22) |
| | (SEQ ID NO: 16) | (SEQ ID NO: 18) | TAVYYC | |
| | | | (SEQ ID NO: 20) | |
| HLkin-1 | QVQLVQSGGGLVQ | MHWVRQAPGK | GYADSVKGRFTVSRDN | WGKGTTVTVSS |
| | PGRSLRLSCAAS | GLEWVSG | AKNSLYLQMNSLRAED | (SEQ ID NO: 22) |
| | (SEQ ID NO: 16) | (SEQ ID NO: 18) | TAVYYC | |
| | | | (SEQ ID NO: 20) | |
| HLki n-1 | QVQLVQSGGGLVQ | MHWVRQAPGK | GYADSVKGRFTVSRDN | WGKGTTVTVSS |
| HL-7 | PGRSLRLSCAAS | GLEWVSG | AKNSLYLQMNSLRAED | (SEQ ID NO: 22) |
| mut2 | (SEQ ID NO: 16) | (SEQ ID NO: 18) | TAVYYC | |
| | | | (SEQ ID NO: 20) | |
| HLkin-1 | QVQLVQSGGGLVQ | MHWVRQAPGK | GYADSVKGRFTVSRDN | WGKGTTVTVSS |
| HL-7 | PGRSLRLSCAAS | GLEWVSG | AKNSLYLQMNSLRAED | (SEQ ID NO: 22) |
| HL-14 | (SEQ ID NO: 16) | (SEQ ID NO: 18) | TAVYYC | |
| mut3 | | | (SEQ ID NO: 20) | |

TABLE 13B
Light chain (V_{L}) framework regions (FRs) of the PD-1 antibodies

| Seq ID | VL FR1 | VL FR2 | VL FR3 | VL FR4 |
|---|---|---|---|---|
| P4-B3 | QPGLTQPPSASGTP | VNWYQQFPGKA | QRPSGVPDRFSASKSG | FGGGTKLTVL |
| | GQRVTISCSGS | PKLLIF | TSASLAISGLQSEDEAD | (SEQ ID NO: 29) |
| | (SEQ ID NO: 24) | (SEQ ID NO: 26) | YYC | |
| | | | (SEQ ID NO: 28) | |
| P4-B7 | LPVLTQPPSASGTP | VHWYQQLPGTA | QRPSGVPDRFSGSKSG | FGGGTKLTVL |
| | GQRVTISCTGS | PKLLIY | TSASLAISGLQSEDEAD | (SEQ ID NO: 29) |
| | FGGGTKLTVL | (SEQ ID NO: 38) | YYC | |
| | (SEQ ID NO: 36) | | (SEQ ID NO: 40) | |
| PD1#2 | QPGLTQPPSVSKGL | AAWLQQHQGHP | NRPSGISERFSASRSGN | FGGGTKLTVL |
| | RQTATLICTGS | PKLLAY | TASLTIIGLQPEDEGDY | (SEQ ID NO: 29) |
| | (SEQ ID NO: 48) | (SEQ ID NO: 50) | YC | |
| | | | (SEQ ID NO: 52) | |
| PD1#3 | NFMLTQPHSVSESP | VQWYQQRPGSS | QRPSGVPDRFSGSIDS | FGGGTKLTVL |
| | GKTVTISCTRN | PTTVIY | SSNSASLTISGLKTEDE | (SEQ ID NO: 29) |
| | (SEQ ID NO: 60) | (SEQ ID NO: 62) | ADYYC | |
| | | | (SEQ ID NO: 64) | |
| PD1#13 | QPGLTQPPSVPVAP | VHWYQQKPGQA | DRPSGIPERFSGSNSG | FGGGTKLTVL |
| | GQTARITCGGN | PVLVVY | NTATLTISRVEAGDEA | (SEQ ID NO: 29) |
| | (SEQ ID NO: 72) | (SEQ ID NO: 74) | DYYC | |
| | | | (SEQ ID NO: 76) | |
| HL-7 | QPGLTQPPSASGTP | VNWYQQFPGKA | QRPSGVPDRFSASKSG | FGGGTKLTVL |
| | GQRVTISCSGS | PKLLIF | TSASLAISGLQSEDEAD | (SEQ ID NO: 29) |
| | (SEQ ID NO: 24) | (SEQ ID NO: 26) | YYC | |
| | | | (SEQ ID NO: 28) | |
| HL-14 | QPGLTQPPSASGTP | VNWYQQFPGKA | QRPSGVPDRFSASKSG | FGGGTKLTVL |
| | GQRVTISCSGS | PKLLIF | TSASLAISGLQSEDEAD | (SEQ ID NO: 29) |
| | (SEQ ID NO: 24) | (SEQ ID NO: 26) | YYC | |
| | | | (SEQ ID NO: 28) | |
| HLkin-1 | QPGLTQPPSASGTP | VNWYQQFPGKA | QRPSGVPDRFSASKSG | FGGGTKLTVL |
| | GQRVTISCSGS | PKLLIF | TSASLAISGLQSEDEAD | (SEQ ID NO: 29) |
| | (SEQ ID NO: 24) | (SEQ ID NO: 26) | YYC | |
| | | | (SEQ ID NO: 28) | |
| HLkin-1 | QPGLTQPPSASGTP | VNWYQQFPGKA | QRPSGVPDRFSASKSG | FGGGTKLTVL |
| HL-7 | GQRVTISCSGS | PKLLIF | TSASLAISGLQSEDEAD | (SEQ ID NO: 29) |
| mut2 | (SEQ ID NO: 24) | (SEQ ID NO: 26) | YYC | |
| | | | (SEQ ID NO: 28) | |
| HLkin-1 | QPGLTQPPSASGTP | VNWYQQFPGKA | QRPSGVPDRFSASKSG | FGGGTKLTVL |
| HL-7 | GQRVTISCSGS | PKLLIF | TSASLAISGLQSEDEAD | (SEQ ID NO: 29) |
| HL-14 mut3 | (SEQ ID NO: 24) | (SEQ ID NO: 26) | YYC | |
| | | | (SEQ ID NO: 28) | |

The PD-1 antibodies described herein bind to PD-1. In one embodiment, the PD-1 antibodies have high affinity and high specificity for PD-1. Some embodiments also feature antibodies that have a specified percentage identity or similarity to the amino acid or nucleotide sequences of the anti-PD-1 antibodies described herein. For example, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the anti-PD-1 antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the anti-PD-1 antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the present invention.

“Polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It may be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art. Such conservatively modified variants of the anti-PD-1 antibodies disclosed herein can exhibit increased cross-reactivity to PD-1 in comparison to an unmodified PD-1 antibody.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

Antibodies

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment”, as used herein, is a portion of an antibody such as F_{(ab′)2}, F_{(ab)2}, F_{ab}′, F_{ab}, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” refers to a fusion protein of the variable regions of the heavy (V_{H}) and light chains (V_{L}) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. (See Huston et al. (1988) Proc Nat Acad Sci USA 85(16):5879-5883). In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases in order to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” VH and VL regions, which contain the CDRs, as well as frameworks (FRs) of the PD-1 antibodies are shown in Table 1A-Table 15B.

The six CDRs present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, the FR regions, show less inter-molecular variability. The framework regions largely adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| CDR | Kabat Numbering | Chothia Numbering |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10⁻¹⁷ residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

As used herein, the term “epitope” can include any protein determinant capable of specific binding to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. Epitopic determinants usually consist of chemically active surface groupings of molecules such as amino acids or sugar side chains and usually have specific three dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3). In one embodiment, the antibodies can be directed to PD-1 (having Genbank accession no. NP_005009; 288 amino acid residues in length), comprising the amino acid sequence of SEQ ID NO: XX:

| | |
|---|---|
| 1 | MQIPQAPWPV VWAVLQLGWR PGWFLDSPDR PWNPPTFSPA LLVVTEGDNA TFTCSFSNTS |
| 61 | ESFVLNWYRM SPSNQTDKLA AFPEDRSQPG QDCRFRVTQL PNGRDFHMSV VRARRNDSGT |
| 121 | YLCGAISLAP KAQIKESLRA ELRVTERRAE VPTAHPSPSP RPAGQFQTLV VGVVGGLLGS |
| 181 | LVLLVWVLAV ICSRAARGTI GARRTGQPLK EDPSAVPVFS VDYGELDFQW REKTPEPPVP |
| 241 | CVPEQTEYAT IVFPSGMGTS SPARRGSADG PRSAQPLRPE DGHCSWPL |

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the equilibrium binding constant (K_{D}) of the interaction, wherein a smaller K_{D} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361: 186-87 (1993)). The ratio of K_{off}/Kₒₙ enables the cancellation of all parameters not related to affinity, and is equal to the equilibrium binding constant, K_{D}. (See, generally, Davies et al. (1990) Annual Rev Biochem 59:439-473). An antibody of the present invention can specifically bind to a PD-1 epitope when the equilibrium binding constant (K_{D}) is ≤1 μM, ≤10 μM, ≤10 nM, ≤10 pM, or ≤100 pM to about 1 pM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3E-11 M, while in other embodiments the K_{D} is about 3E-12 M. In some embodiments, the K_{D} is about 6E-II M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the PD-1 antibody can be monovalent or bivalent, and comprises a single or double chain. Functionally, the binding affinity of the PD-1 antibody is within the range of 10⁻⁵ M to 10⁻¹² M. For example, the binding affinity of the PD-1 antibody is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M, or from 10⁻⁵ M to 10⁻⁶ M.

A PD-1 protein of the invention, or a derivative, fragment, analog, homolog or ortholog thereof, can be utilized as an immunogen in the generation of antibodies that immunospecifically bind these protein components, e.g., amino acid residues comprising SEQ ID NO: X. A PD-1 protein or a derivative, fragment, analog, homolog, or ortholog thereof, coupled to a proteoliposome can be utilized as an immunogen in the generation of antibodies that immunospecifically bind these protein components.

Those skilled in the art will recognize that it is possible to determine, without undue experimentation, if a human monoclonal antibody has the same specificity as a human monoclonal antibody of the invention by ascertaining whether the former prevents the latter from binding to PD-1. For example, if the human monoclonal antibody being tested competes with the human monoclonal antibody of the invention, as shown by a decrease in binding by the human monoclonal antibody of the invention, then it is likely that the two monoclonal antibodies bind to the same, or to a closely related, epitope.

Another way to determine whether a human monoclonal antibody has the specificity of a human monoclonal antibody of the invention is to pre-incubate the human monoclonal antibody of the invention with the PD-1 protein, with which it is normally reactive, and then add the human monoclonal antibody being tested to determine if the human monoclonal antibody being tested is inhibited in its ability to bind PD-1. If the human monoclonal antibody being tested is inhibited then, in all likelihood, it has the same, or functionally equivalent, epitopic specificity as the monoclonal antibody of the invention. Screening of human monoclonal antibodies of the invention can be also carried out by utilizing PD-1 and determining whether the test monoclonal antibody is able to neutralize PD-1.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, N.Y., incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are desired, or spleen cells or lymph node cells can be used if non-human mammalian sources are desired. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, particularly myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines that are useful are those that fuse efficiently, support stable high level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, Calif.) and the American Type Culture Collection (Manassas, Va.). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies, for example, are antibody molecules in which the entire sequence of both the light chain and the heavy chain, including the CDRs, arise from human genes. Such antibodies are termed “human antibodies” or “fully human antibodies”. “Humanized antibodies” can be antibodies from non-human species whose light chain and heavy chain protein sequences have been modified to increase their similarity to antibody variants produced in humans. Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, for example improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) For example, the non-human part of the antibody (such as the CDR(s) of a light chain and/or heavy chain) can bind to the target antigen. A humanized monoclonal antibody can also be referred to a “human monoclonal antibody” herein.

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (commonly rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20).

Human monoclonal antibodies, such as fully human and humanized antibodies, can be prepared by using trioma technique; the human B-cell hybridoma technique (see Kozbor, et al, 1983 Immunol Today 4: 72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, antibodies can also be produced using other techniques, including phage display libraries. (See Hoogenboom and Winter, J. Mol. Biol, 227:381 (1991); Marks et al., J. Mol. Biol, 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al, Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies can additionally be produced using transgenic nonhuman animals which are modified so as to produce fully human antibodies rather than the animal's endogenous antibodies in response to challenge by an antigen. (See, PCT publication no. WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. A non-limiting example of such a nonhuman animal is a mouse, and is termed the Xenomouse™ as disclosed in PCT publication nos. WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules. Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, N.Y.) can be engaged to provide human antibodies directed against a selected antigen using technology similar to that described above.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99/53049.

The antibody of interest can also be expressed by a vector containing a DNA segment encoding the single chain antibody described above. Vectors include, but are not limited to, chemical conjugates such as described in WO 93/64701, which has targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used, and include moloney murine leukemia viruses. DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87: 1149 (1990), Adenovirus Vectors (see LeGal LaSalle et al, Science, 259:988 (1993); Davidson, et al, Nat. Genet 3: 219 (1993); Yang, et al, J. Virol. 69:2004 (1995) and Adeno-associated Virus Vectors (see Kaplitt, M. G. et al, Nat. Genet. 8: 148 (1994).

Pox viral vectors introduce the gene into the cells cytoplasm. Avipox virus vectors result in only a short term expression of the nucleic acid. Adenovirus vectors, adeno-associated virus vectors, and herpes simplex virus (HSV) vectors can be used for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaP0₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91: 2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of PD-1 in a sample. The antibody can also be used to try to bind to and disrupt a PD-1 activity.

Techniques can be adapted for the production of single-chain antibodies specific to an antigenic protein of the invention (See e.g., U.S. Pat. No. 4,946,778). In addition, methods can be adapted for the construction of Fab expression libraries (See e.g., Huse, et al, 1989 Science 246: 1275-1281) to allow rapid and effective identification of monoclonal FA fragments with the desired specificity for a protein or derivatives, fragments, analogs or homologs thereof. Antibody fragments that contain the idiotypes to a protein antigen can be produced by techniques known in the art including, but not limited to: (i) an F_{(ab′)2} fragment produced by pepsin digestion of an antibody molecule; (ii) an Fab fragment generated by reducing the disulfide bridges of an F_{(ab′)2} fragment; (iii) an Fab fragment generated by the treatment of the antibody molecule with papain and a reducing agent and (iv) Fv fragments.

Heteroconjugate antibodies are also within the scope of the present invention. Heteroconjugate antibodies are composed of two covalently joined antibodies. Such antibodies can, for example, target immune system cells to unwanted cells (see U.S. Pat. No. 4,676,980), and for treatment of HIV infection (See PCT Publication Nos. WO91/00360; WO92/20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of the invention can be modified with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176: 1 191-1 195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al, Anti-Cancer Drug Design, 3: 219-230 (1989)).

In certain embodiments, an antibody of the invention can comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter halt-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be desired include those applications in which localization to the brain, kidney, and/or liver is desired. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In some embodiments, mutations are introduced to the constant regions of the mAb such that the antibody dependent cell-mediated cytotoxicity (ADCC) activity of the mAb is altered. For example, the mutation is an LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In other embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In a particular embodiment, the antibody comprises an Fc variant with an amino acid substitution at amino acid position 228 or 299 (EU numbering). In more particular embodiments, the antibody comprises an IgG₁ or IgG4 constant region comprising an S228P and a T299A mutation (EU numbering).

Exemplary amino acid substitutions which confer reduced or altered glycosylation are described in PCT Publication No, WO05/018572, which is incorporated by reference herein in its entirety. In some embodiments, the antibodies of the invention, or fragments thereof, are modified to eliminate glycosylation. Such antibodies, or fragments thereof, can be referred to as “agly” antibodies, or fragments thereof, (e.g. “agly” antibodies). While not wishing to be bound by theory “agly” antibodies, or fragments thereof, can have an improved safety and stability profile in vivo. Exemplary agly antibodies, or fragments thereof, comprise an aglycosylated Fc region of an IgG₄ antibody which is devoid of Fc-effector function thereby eliminating the potential for Fc mediated toxicity to the normal vital tissues and cells that express PD-1. In yet other embodiments, antibodies of the invention, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

The invention also is directed to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See PCT Publication No. WO94/11026, and U.S. Pat. No. 5,736,137).

Those of ordinary skill in the art understand that a large variety of possible moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133: 1335-2549 (1984); Jansen et al., Immunological Reviews 62: 185-216 (1982); and Vitetta et al, Science 238: 1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS(-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al, Proc. Natl. Acad. Sci. USA, 82: 3688 (1985); Hwang et al, Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al, J. Biol. Chem., 257: 286-288 (1982) via a disulfide-interchange reaction.

Bi-Specific Antibodies

A bi-specific antibody (bsAb) is an antibody comprising two variable domains or scFv units such that the resulting antibody recognizes two different antigens. The present invention provides for bi-specific antibodies that recognize PD-1 and a second antigen. Exemplary second antigens include tumor associated antigens (e.g., LINGO1), cytokines, and cell surface receptors. Non-limiting examples of second antigens include CTLA-4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM (or BTLA), CD47 and CD73. Different format of bispecific antibodies are also provided herein. In some embodiments, each of the anti-PD1 fragment and the second fragment is each independently selected from a Fab fragment, a single-chain variable fragment (scFv), or a single-domain antibody. In some embodiments, the bispecific antibody further includes a Fc fragment. A bi-specific antibody of the present invention comprises a heavy chain and a light chain combination or scFv of the PD-1 antibodies disclosed herein.

Bi-specific antibodies of the present invention can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds.

In another embodiment, the bi-specific antibody is constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens are mixed to promote heteroligation pairing, which is mediated through the engineered “knob into holes” of the CH3 domains.

In another embodiment, the bi-specific antibody can be constructed through exchange of heavy-light chain dimers from two or more different antibodies to generate a hybrid antibody where the first heavy-light chain dimer recognizes PD-1 and the second heavy-light chain dimer recognizes a second antigen. The mechanism for heavy-light chain dimer is similar to the formation of human IgG₄, which also functions as a bispecific molecule. Dimerization of IgG heavy chains is driven by intramolecular force, such as the pairing the CH3 domain of each heavy chain and disulfide bridges. Presence of a specific amino acid in the CH3 domain (R409) has been shown to promote dimer exchange and construction of the IgG₄ molecules. Heavy chain pairing is also stabilized further by interheavy chain disulfide bridges in the hinge region of the antibody. Specifically, in IgG₄, the hinge region contains the amino acid sequence Cys-Pro-Ser-Cys (in comparison to the stable IgGl hinge region which contains the sequence Cys-Pro-Pro-Cys) at amino acids 226-230. This sequence difference of Serine at position 229 has been linked to the tendency of IgG₄ to form intrachain disulfides in the hinge region (Van der Neut Kolfschoten, M. et al, 2007, Science 317: 1554-1557 and Labrijn, A. F. et al, 2011, Journal of Immunol 187:3238-3246).

Therefore, bi-specific antibodies of the present invention can be created through introduction of the R409 residue in the CH3 domain and the Cys-Pro-Ser-Cys sequence in the hinge region of antibodies that recognize PD-1 or a second antigen, so that the heavy-light chain dimers exchange to produce an antibody molecule with one heavy-light chain dimer recognizing PD-1 and the second heavy-light chain dimer recognizing a second antigen, wherein the second antigen is any antigen disclosed herein. Known IgG₄ molecules can also be altered such that the heavy and light chains recognize PD-1 or a second antigen, as disclosed herein. Use of this method for constructing the bi-specific antibodies of the present invention can be beneficial due to the intrinsic characteristic of IgG₄ molecules wherein the Fc region differs from other IgG subtypes in that it interacts poorly with effector systems of the immune response, such as complement and Fc receptors expressed by certain white blood cells. This specific property makes these IgG₄-based bi-specific antibodies attractive for therapeutic applications, in which the antibody is required to bind the target(s) and functionally alter the signaling pathways associated with the target(s), however not trigger effector activities.

In some embodiments, mutations are introduced to the constant regions of the bsAb such that the antibody dependent cell-mediated cytotoxicity (ADCC) activity of the bsAb is altered. For example, the mutation is an LALA mutation in the CH2 domain. In one aspect, the bsAb contains mutations on one scFv unit of the heterodimeric bsAb, which reduces the ADCC activity. In another aspect, the bsAb contains mutations on both chains of the heterodimeric bsAb, which completely ablates the ADCC activity. For example, the mutations introduced one or both scFv units of the bsAb are LALA mutations in the CH2 domain. These bsAbs with variable ADCC activity can be optimized such that the bsAbs exhibits maximal selective killing towards cells that express one antigen that is recognized by the bsAb, however exhibits minimal killing towards the second antigen that is recognized by the bsAb.

The bi-specific antibodies disclosed herein can be useful in treatment of chronic infections, diseases, or medical conditions, for example, cancer.

Use of Antibodies Against PD-1

Antibodies of the invention specifically binding a PD-1 protein, or a fragment thereof, can be administered for the treatment a PD-1 associated disease or disorder. A “PD-1-associated disease or disorder” includes disease states and/or symptoms associated with a disease state, where increased levels of PD-1 and/or activation of cellular signaling pathways involving PD-1 are found. Exemplary PD-1-associated diseases or disorders include but are not limited to diseases where T cells are suppressed, such as in cancer and infectious diseases. In some embodiments, the infectious disease can be caused by a microorganism, such as a DNA virus, RNA virus, or reverse transcribing virus. Non-limiting examples of viruses include Adenovirus, Coxsackievirus, Epstein-Barr virus, Hepatitis A virus, Hepatitis B virus, Hepatitis C virus, Herpes simplex virus, type 1, Herpes simplex virus, type 2, Cytomegalovirus, Human herpesvirus, type 8, HIV, Influenza virus, Measles virus, Mumps virus, Human papillomavirus, Parainfluenza virus, Poliovirus, Rabies virus, Respiratory syncytial virus, Rubella virus, Varicella-zoster virus. In some embodiments, the infectious disease can be caused by a microorganism, such as a Gram positive bacterium, a Gram negative bacterium, a protozoa, or a fungus.

Non-limiting examples of disease-causing bacteria include: Bacillus anthracis, Bacillus cereus, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheria, Enterococcus faecalis, Enterococcus faecium, Escherichia coli, Francisella tularensis, Haemophilus influenza, Helicobacter pylon, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitides, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholera, Yersinia pestis, Yersinia enterocolitica, Yersinia pseudotuberculosis.

Non-limiting examples of disease causing protozoa include: Plasmodium falciparum (malaria), Toxoplasma gondii (toxoplasmosis), Leishmania species (leishmaniases), Trypanosoma brucei (African sleeping sickness), Trypanosoma cruzi (Chagas disease), and Giardia intestinalis (giardiasis).

Non-limiting examples of disease causing fungi include Candida albicans, Aspergillus fumigatus, Aspergillus flavus, Cryptococcus neoformans, Cryptococcus gattii, Histoplasma capsulatum, Pneumocystis carinii, Stachybotrys chartarum.

Antibodies of the invention, including bi-specific, polyclonal, monoclonal, humanized and fully human antibodies, can be used as therapeutic agents. Such agents will generally be employed to treat cancer in a subject, increase vaccine efficiency or augment a natural immune response. An antibody preparation, for example, one having high specificity and high affinity for its target antigen, is administered to the subject and will generally have an effect due to its binding with the target. Administration of the antibody can abrogate or inhibit or interfere with an activity of the PD-1 protein.

Antibodies of the invention specifically binding a PD-1 protein or fragment thereof can be administered for the treatment of a cancer in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R. Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

A therapeutically effective amount of an antibody of the invention can be the amount needed to achieve a therapeutic objective. As noted herein, this can be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. The dosage administered to a subject (e.g., a patient) of the antigen-binding polypeptides described herein is typically 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90: 7889-7893 (1993)). The formulation can also contain more than one active compound as necessary for the particular indication being treated, for example, those with complementary activities that do not adversely affect each other. Alternatively, or in addition, the composition can comprise an agent that enhances its function, such as, for example, a cytotoxic agent, cytokine (e.g. IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of PD-1 (or a protein fragment thereof) in a sample. For example, the antibody can contain a detectable label. Antibodies can be polyclonal or monoclonal. An intact antibody, or a fragment thereof (e.g., F_{ab}, scFv, or F_{(ab)2}) can be used. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations.

Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Antibodies directed against a PD-1 protein (or a fragment thereof) can be used in methods known within the art relating to the localization and/or quantitation of a PD-1 protein (e.g., for use in measuring levels of the PD-1 protein within appropriate physiological samples, for use in diagnostic methods, for use in imaging the protein, and the like). In a given embodiment, antibodies specific to a PD-1 protein, or derivative, fragment, analog or homolog thereof, that contain the antibody derived antigen binding domain, are utilized as pharmacologically active compounds (referred to herein as “therapeutics”).

An antibody of the invention specific for a PD-1 protein can be used to isolate a PD-1 polypeptide by standard techniques, such as immunoaffinity, chromatography or immunoprecipitation. Antibodies directed against a PD-1 protein (or a fragment thereof) can be used diagnostically to monitor protein levels in tissue as part of a clinical testing procedure, e.g., to, for example, determine the efficacy of a given treatment regimen.

Detection can be facilitated by coupling (i.e., physically linking) the antibody to a detectable substance. Examples of detectable substances include, but are not limited to, various enzymes, prosthetic groups, fluorescent materials, luminescent materials, bioluminescent materials, and radioactive materials. Non-limiting examples of suitable enzymes include horseradish peroxidase, alkaline phosphatase, 0-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P or ³H.

The antibodies or agents of the invention (also referred to herein as “active compounds”), and derivatives, fragments, analogs and homologs thereof, can be incorporated into pharmaceutical compositions suitable for administration. Such pharmaceutical compositions can comprise the antibody or agent and a pharmaceutically acceptable carrier. As used herein, the term “pharmaceutically acceptable carrier” can include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents can be included, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. For example, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

Methods of Treatment

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

The invention provides for both prophylactic and therapeutic methods of treating a subject at risk of (or susceptible to) a cancer (for example, if an early detection cancer biomarker is identified in such a subject), or other cell proliferation-related diseases or disorders. Such diseases or disorders include but are not limited to, e.g., those diseases or disorders associated with aberrant expression of PD-1. For example, the methods are used to treat, prevent or alleviate a symptom of cancer. In an embodiment, the methods are used to treat, prevent or alleviate a symptom of a solid tumor. Non-limiting examples of other tumors that can be treated by embodiments herein comprise lung cancer, ovarian cancer, prostate cancer, colon cancer, cervical cancer, brain cancer, skin cancer, liver cancer, pancreatic cancer or stomach cancer. Additionally, the methods of the invention can be used to treat hematologic cancers such as leukemia and lymphoma. Alternatively, the methods can be used to treat, prevent or alleviate a symptom of a cancer that has metastasized. For example, cancers that can be treated or prevented or for which symptoms can be alleviated include B-cell chronic lymphocytic leukemia (CLL), non-small-cell lung cancer, melanoma, ovarian cancer, lymphoma, or renal-cell cancer. Cancers that can also be treated or prevented or for which symptoms can be alleviated include those solid tumors with a high mutation burden and WBC in filtrate. Cancers that can be treated or prevented or for which symptoms can be alleviated further include cancers where signals in the PD-1/PD-L1 axis have been modulated, cancers which include (but are not limited to) breast cancer, lung cancer (e.g., non-small cell lung cancer or lung adenocarcinoma), gastric cancer, colorectal cancer, bladder cancer, pancreatic cancer, prostate cancer, esophageal squamous cell carcinoma, nasopharyngeal carcinoma, and liquid tumors with the PD1/PDL1 axis active (such as diffuse large B-cell lymphoma (DLBCL) and B-cell chronic lymphocytic leukemia (B-CLL)) (see e.g., Han et al., PD-1/PD-L1 pathway: current researches in cancer, Am J Cancer Res 2020; 10(3):727-742).

Accordingly, in one aspect, the invention provides methods for preventing, treating or alleviating a symptom cancer or a cell proliferative disease or disorder in a subject by administering to the subject a monoclonal antibody, scFv antibody or bi-specific antibody of the invention. For example, an anti-PD-1 antibody can be administered in therapeutically effective amounts.

Subjects at risk for cancer or cell proliferation-related diseases or disorders can include patients who have a family history of cancer or a subject exposed to a known or suspected cancer-causing agent. Administration of a prophylactic agent can occur prior to the manifestation of cancer such that the disease is prevented or, alternatively, delayed in its progression.

In another aspect, tumor cell growth is inhibited by contacting a cell with an anti-PD-1 antibody of the invention. The cell can be any cell that expresses PD-1.

The invention further provides for both prophylactic and therapeutic methods of treating a subject at risk of (or susceptible to) a chronic or acute viral, bacterial or parasitic infection. The invention also provides for therapeutic methods for both prophylactic and therapeutic methods of treating a subject at risk of a disease or disorder or condition associated with T-cell exhaustion or a risk of developing T-cell exhaustion. The invention also provides for therapeutic methods for both prophylactic and therapeutic methods of treating a subject at risk of a disease or disorder or condition associated with T-cell exhaustion or a risk of developing T-cell exhaustion. Such diseases or disorder include, but are not limited to HIV, AIDS, and chronic or acute bacterial, viral or parasitic infections. Other such chronic infections include those caused by, for example, hepatitis B virus (HBV), hepatitis C virus (HCV), herpes simplex virus 1 (HSV-1), H. pylori, or Toxoplasma gondii. Other acute infections included are those caused by, for example, microorganisms, such as a Gram positive bacterium, a Gram negative bacterium, a protozoa, or a fungus, as described herein.

Also included in the invention are methods of increasing or enhancing an immune response to an antigen. An immune response is increased or enhanced by administering to the subject a monoclonal antibody, scFv antibody, or bi-specific antibody of the invention. The immune response is augmented for example by augmenting antigen specific T effector function. The antigen is a viral (e.g. HIV), bacterial, parasitic or tumor antigen. The immune response is a natural immune response. By natural immune response is meant an immune response that is a result of an infection. The infection is a chronic infection. Increasing or enhancing an immune response to an antigen can be measured by a number of methods known in the art. For example, an immune response can be measured by measuring any one of the following: T cell activity, T cell proliferation, T cell activation, production of effector cytokines, and T cell transcriptional profile. Alternatively, the immune response is a response induced due to a vaccination.

Accordingly, in another aspect the invention provides a method of increasing vaccine efficiency by administering to the subject a monoclonal antibody or scFv antibody of the invention and a vaccine. The antibody and the vaccine are administered sequentially or concurrently. The vaccine is a tumor vaccine a bacterial vaccine or a viral vaccine.

Combinatory Methods

Compositions of the invention as described herein can be administered in combination with a chemotherapeutic agent. Chemotherapeutic agents that may be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In additional embodiments, the compositions of the invention as described herein can be administered in combination with cytokines. Cytokines that may be administered with the compositions include, but are not limited to, IL-2, IL-3, IL-4, IL-5, IL-6, IL-7, IL-10, IL-13, IL-15, anti-CD40, CD40L, and TNF-α.

In additional embodiments, the compositions described herein can be administered in combination with other therapeutic or prophylactic regimens, such as, for example, radiation therapy.

In some embodiments, the compositions described herein can be administered in combination with other immunotherapeutic agents. Non-limiting examples of immunotherapeutic agents include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8.

The invention provides for methods of treating cancer in a patient by administering two antibodies that bind to the same epitope of the PD-1 protein or, alternatively, two different epitopes of the PD-1 protein. Alternatively, the cancer can be treated by administering a first antibody that binds to PD-1 and a second antibody that binds to a protein other than PD-1. In other embodiments, the cancer can be treated by administering a bispecific antibody that binds to PD-1 and that binds to a protein other than PD-1. For example, the other protein other than PD-1 can include, but is not limited to, IL-2, IL-2R, IL-15, IL-15R, IL-7, IL-7R, IL-21, or IL-21R. For example, the other protein other than PD-1 is a tumor-associated antigen; the other protein other than PD-1 can also be a cytokine.

In some embodiments, the invention provides for the administration of an anti-PD-1 antibody alone or in combination with an additional antibody that recognizes another protein other than PD-1, with cells that are capable of effecting or augmenting an immune response. For example, these cells can be peripheral blood mononuclear cells (PBMC), or any cell type that is found in PBMC, e.g., cytotoxic T cells, macrophages, and natural killer (NK) cells.

Additionally, the invention provides administration of an antibody that binds to the PD-1 protein and an anti-neoplastic agent, such a small molecule, a growth factor, a cytokine or other therapeutics including biomolecules such as peptides, peptidomimetics, peptoids, polynucleotides, lipid-derived mediators, small biogenic amines, hormones, neuropeptides, and proteases. Small molecules include, but are not limited to, inorganic molecules and small organic molecules. Suitable growth factors or cytokines include an IL-2, GM-CSF, and TNF-alpha. Small molecule libraries are known in the art. (See, Lam, Anticancer Drug Des., 12: 145, 1997.)

Chimeric Antigen Receptor (CAR) T-Cell Therapies

Cellular therapies, such as chimeric antigen receptor (CAR) T-cell therapies, are also provided herein. CAR T-cell therapies redirect a patient's T-cells to kill tumor cells by the exogenous expression of a CAR on a T-cell, for example. A CAR can be a membrane spanning fusion protein that links the antigen recognition domain of an antibody to the intracellular signaling domains of the T-cell receptor and co-receptor. A suitable cell can be used, that can secrete an anti-PD-1 antibody of the present invention (or alternatively engineered to express an anti-PD-1 antibody as described herein to be secreted). The anti-PD-1 “payloads” to be secreted, can be, for example, minibodies, ScFvs, IgG molecules, bispecific fusion molecules, and other antibody fragments as described herein.

Solid tumors offer unique challenges for CAR-T therapies. Some barriers to CAR-T effectiveness in solid tumors include heterogeneous antigen expression, insufficient tissue homing, activation, persistence, and the immunosuppressive tumor microenvironment. Unlike blood cancers, tumor-associated target proteins are overexpressed between the tumor and healthy tissue resulting in on-target/off-tumor T-cell killing of healthy tissues. Furthermore, immune repression in the tumor microenvironment (TME) limits the activation of CAR-T cells towards killing the tumor. Upon such contact or engineering, the cell can then be introduced to a cancer patient in need of a treatment. The cancer patient may have a cancer of any of the types as disclosed herein. The cell (e.g., a T cell) can be, for instance, a tumor-infiltrating T lymphocyte, a CD4+ T cell, a CD8+ T cell, or the combination thereof, without limitation.

Exemplary CARs and CAR factories useful in aspects of the invention include those disclosed in, for example, PCT/US2015/067225 and PCT/US2019/022272, each of which are hereby incorporated by reference in their entireties. For example, CAR-T cells can be generated according to methods known in the art using lentivirus systems (via transduction), retrovirus systems (via transfection (electroporation)), and transposon systems (via PiggyBac). Useful for promoters for payloads that can be used in the generating of CAR-Ts include, for example, constitutive promoters (where the promoter is the same as for CAR-T, such as EF1a then IRES or 2A); inducible promoters (where the promoter is different from the promoter for CAR-T, such as NFAT, IL-2 prom); and genetically engineered promoters (such as a PD-1 locus “knock in” of cytokine and/or a promoter that is under the control of an endogenous promoter). In one embodiment, the PD-1 antibodies discussed herein can be used in the construction of multi-specific antibodies or as the payload for a CAR-T cell. For example, in one embodiment, the anti-PD-1 antibodies discussed herein can be used for the targeting of the CARS (i.e., as the targeting moiety). In one embodiment, the anti-PD-1 antibodies discussed herein can be used as a payload to be secreted by a CAR-T cell. In another embodiment, the anti-PD-1 antibodies discussed herein can be used as the targeting moiety, and a different PD-1 antibody that targets a different epitope can be used as the payload. In another embodiment, the payload can be an immunomodulatory antibody payload. In some embodiments, the PD-1 antibodies as described herein for use in CAR-T compositions are not high-affinity PD-1 antibodies (for example, so that the antibody does not bind strongly to its PD-1 target). For example, the PD-1 antibodies described herein can be used as a payload secreted by the CAR-T cell, with the two targeting moieties (for example, tumor-associated surface antigens) selected for a specific cancer (i.e. MSLN and MUC1 for ovarian cancer). Non-limiting examples of a tumor-associated surface antigen include ErbB2 (HER2/neu), carcinoembryonic antigen (CEA), epithelial cell adhesion molecule (EpCAM), epidermal growth factor receptor (EGFR), MUC1, MSLN, CD19, CD20, CD30, CD40, CD22, RAGE-1, MN-CA IX, RET1, RET2 (AS), prostate specific antigen (PSA), TAG-72, PAP, p53, Ras, prostein, PSMA, survivin, 9D7, prostate-carcinoma tumor antigen-1 (PCTA-1), GAGE, MAGE, mesothelin, β-catenin, TGF-βRII, BRCA1/2, SAP-1, HPV-E6, HPV-E7 (see also, PCT/US2015/067225 and PCT/US2019/022272 for additional tumor-associated surface antigens, which are incorporated by reference in their entireties).

Diagnostic Assays

The anti-PD-1 antibodies can be used diagnostically to, for example, monitor the development or progression of cancer as part of a clinical testing procedure to, e.g., determine the efficacy of a given treatment and/or prevention regimen.

In some aspects, for diagnostic purposes, the anti-PD-1 antibody of the invention is linked to a detectable moiety, for example, so as to provide a method for detecting a cancer cell in a subject at risk of or suffering from a cancer.

The detectable moieties can be conjugated directly to the antibodies or fragments, or indirectly by using, for example, a fluorescent secondary antibody. Direct conjugation can be accomplished by standard chemical coupling of, for example, a fluorophore to the antibody or antibody fragment, or through genetic engineering. Chimeras, or fusion proteins can be constructed which contain an antibody or antibody fragment coupled to a fluorescent or bioluminescent protein. For example, Casadei, et al, (Proc Natl Acad Sci USA. 1990 March; 87(6):2047-51) describe a method of making a vector construct capable of expressing a fusion protein of aequorin and an antibody gene in mammalian cells.

As used herein, the term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject (such as a biopsy), as well as tissues, cells and fluids present within a subject. That is, the detection method of the invention can be used to detect cells that express PD-1 in a biological sample in vitro as well as in vivo. For example, in vitro techniques for detection of PD-1 include enzyme linked immunosorbent assays (ELISAs), Western blots, immunoprecipitations, and immunofluorescence. Furthermore, in vivo techniques for detection of PD-1 include introducing into a subject a labeled anti-PD-1 antibody. For example, the antibody can be labeled with a radioactive marker whose presence and location in a subject can be detected by standard imaging techniques.

In the case of “targeted” conjugates, that is, conjugates which contain a targeting moiety—a molecule or feature designed to localize the conjugate within a subject or animal at a particular site or sites, localization can refer to a state when an equilibrium between bound, “localized”, and unbound, “free” entities within a subject has been essentially achieved. The rate at which such equilibrium is achieved depends upon the route of administration. For example, a conjugate administered by intravenous injection can achieve localization within minutes of injection. On the other hand, a conjugate administered orally can take hours to achieve localization. Alternatively, localization can simply refer to the location of the entity within the subject or animal at selected time periods after the entity is administered. By way of another example, localization is achieved when an moiety becomes distributed following administration.

It is understood that a reasonable estimate of the time to achieve localization can be made by one skilled in the art. Furthermore, the state of localization as a function of time can be followed by imaging the detectable moiety (e.g., a light-emitting conjugate) according to the methods of the invention, such as with a photodetector device. The “photodetector device” used should have a high enough sensitivity to enable the imaging of faint light from within a mammal in a reasonable amount of time, and to use the signal from such a device to construct an image.

In cases where it is possible to use light-generating moieties which are extremely bright, and/or to detect light-generating fusion proteins localized near the surface of the subject or animal being imaged, a pair of “night-vision” goggles or a standard high-sensitivity video camera, such as a Silicon Intensified Tube (SIT) camera (e.g., from Hammamatsu Photonic Systems, Bridgewater, N.J.), can be used. More typically, however, a more sensitive method of light detection is required.

In extremely low light levels the photon flux per unit area becomes so low that the scene being imaged no longer appears continuous. Instead, it is represented by individual photons which are both temporally and spatially distinct form one another. Viewed on a monitor, such an image appears as scintillating points of light, each representing a single detected photon. By accumulating these detected photons in a digital image processor over time, an image can be acquired and constructed. In contrast to conventional cameras where the signal at each image point is assigned an intensity value, in photon counting imaging the amplitude of the signal carries no significance. The objective is to simply detect the presence of a signal (photon) and to count the occurrence of the signal with respect to its position over time.

At least two types of photodetector devices, described below, can detect individual photons and generate a signal which can be analyzed by an image processor. Reduced-Noise Photodetection devices achieve sensitivity by reducing the background noise in the photon detector, as opposed to amplifying the photon signal. Noise is reduced primarily by cooling the detector array. The devices include charge coupled device (CCD) cameras referred to as “backthinned”, cooled CCD cameras. In the more sensitive instruments, the cooling is achieved using, for example, liquid nitrogen, which brings the temperature of the CCD array to approximately −120° C. “Backthinned” refers to an ultra-thin backplate that reduces the path length that a photon follows to be detected, thereby increasing the quantum efficiency. A particularly sensitive backthinned cryogenic CCD camera is the “TECH 512”, a series 200 camera available from Photometries, Ltd. (Tucson, Ariz.).

“Photon amplification devices” amplify photons before they hit the detection screen. This class includes CCD cameras with intensifiers, such as microchannel intensifiers. A microchannel intensifier typically contains a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Even greater sensitivity can be achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification. An exemplary microchannel intensifier-based single-photon detection device is the C2400 series, available from Hamamatsu.

Image processors process signals generated by photodetector devices which count photons in order to construct an image which can be, for example, displayed on a monitor or printed on a video printer. Such image processors are typically sold as part of systems which include the sensitive photon-counting cameras described above, and accordingly, are available from the same sources. The image processors are usually connected to a personal computer, such as an IBM-compatible PC or an Apple Macintosh (Apple Computer, Cupertino, Calif.), which may or may not be included as part of a purchased imaging system. Once the images are in the form of digital files, they can be manipulated by a variety of image processing programs (such as “ADOBE PHOTOSHOP”, Adobe Systems, Adobe Systems, Mt. View, Calif.) and printed.

In an embodiment, the biological sample contains protein molecules from the test subject. One exemplary biological sample is a peripheral blood leukocyte sample isolated by conventional means from a subject.

The invention also encompasses kits for detecting the presence of PD-1 or a PD-1-expressing cell in a biological sample. For example, the kit can comprise: a labeled compound or agent capable of detecting a cancer or tumor cell (e.g., an anti-PD-1 scFv or monoclonal antibody) in a biological sample; means for determining the amount of PD-1 in the sample; and means for comparing the amount of PD-1 in the sample with a standard. The standard is, in some embodiments, a non-cancer cell or cell extract thereof. The compound or agent can be packaged in a suitable container. The kit can further comprise instructions for using the kit to detect cancer in a sample.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention are further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—PMPL Panning

PD-1 antibodies of the invention (e.g., P4-B3 and P4-B7) were found via PMPL panning. Briefly, PD-1 was expressed genetically fused to a C-terminal C9 tag (TETSQVAPA). Expi293 cells were transiently transfected and then lysed. The lysate was clarified and 1D4 (anti-C9 tag) conjugated magnetic beads were used to capture the PD-1 proteins. The beads were then dialyzed in a lipid solution which allowed for the formation of a lipid bilayer around the bead which simulates the cell membrane and helps in protein stability. These beads were then used for panning.

### Example 2—Minibody Binding Curves

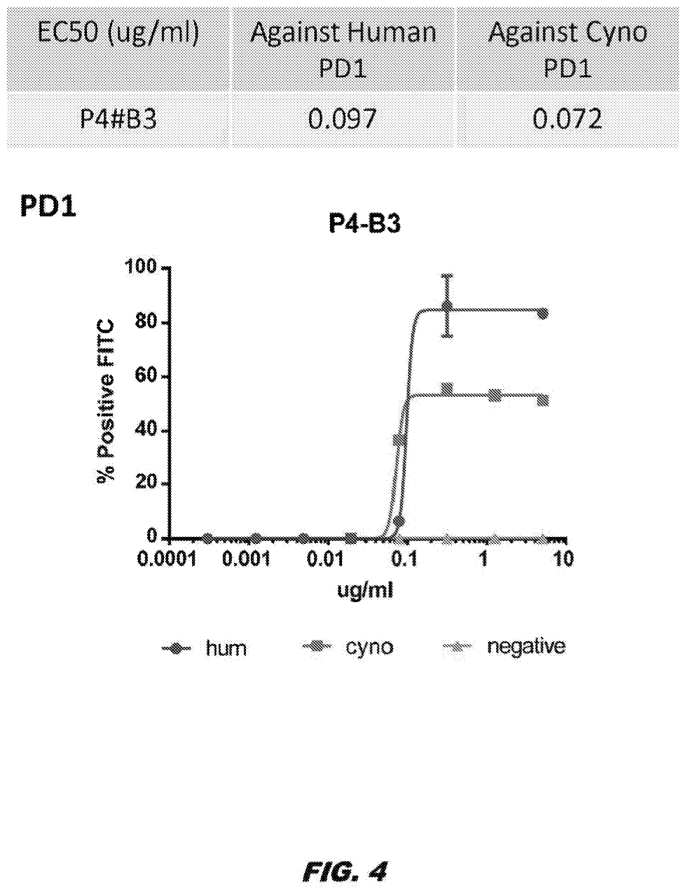

Minibody binding curves were conducted with transfected cells (see FIG. 4). Cells transfected with human or cyno PD1 were used to develop binding curves for P4-B3 minibodies. Human variant was performed in duplicate whereas the negative and cyno were carried out in singlet. Curves were generated with Expi293 cells 48 hours after transfection. Human variants curves were normalized based on expression levels via commercial antibody staining, however the cyno variants were not. Cyno variants were not normalized because the commercial antibodies used are not reported to bind to cyno PD-1.

### Example 3—Octet Binding Curve for Different Antibody Formats of P4-B3

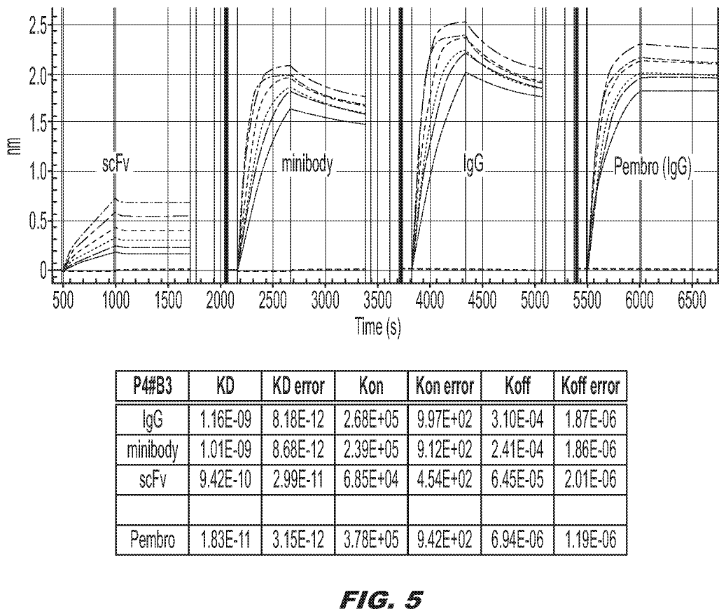

Streptavidin sensors were loaded with 3 ug/ml biotinylated PD-1. The top concentration for all formats of P4-B3 is 50 nM and ¾ serial dilutions were carried out. Kinetic calculations were carried out using the Octet Red software and is shown in FIG. 5. Per EMEA Assessment Report (EMEA/H/C/003820/0000) the reported K_{D} of Pembro is 2.9E-11 M, which is comparable to the results generated for Pembro from the experiment.

### Example 4—PD-L1 Competition Assays

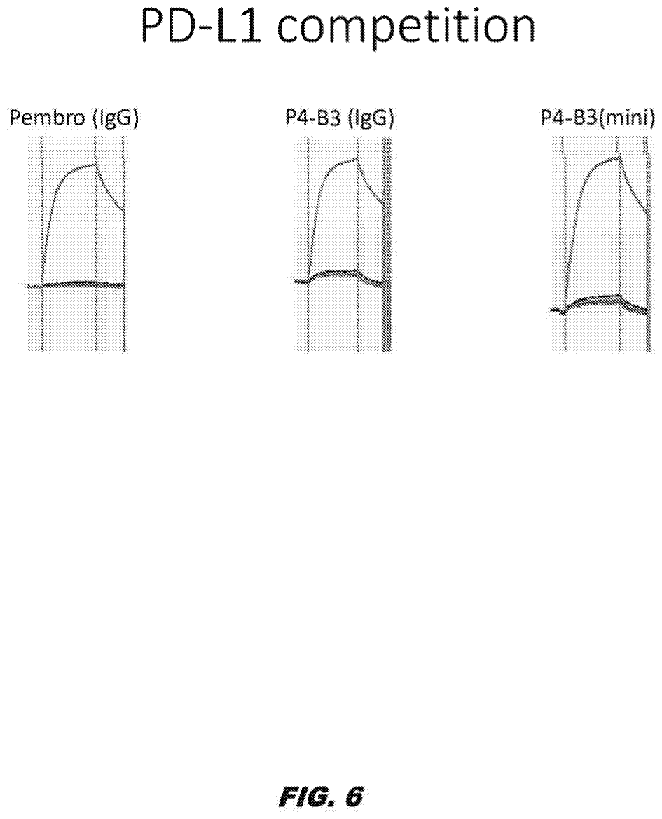

SA sensors were loaded 3 ug/ml PD-1 and then incubated with varying concentrations (50-0 nM) of either Pembro (IgG) or P4-B3 (IgG or minibody) followed by 5 ug/ml PD-L1. In FIG. 6, the red curve has no antibody loaded and represents the maximum amount of PD-L1 binding to the PD-1 functionalized sensor. As shown in FIG. 6, the P4-B3 antibody has a slight shift with the addition of PD-L1 but appears to block a good portion of PD-L1 binding. The curves do not include the antibody loading steps, instead just show the PD-L1 binding step. Original antibody binding steps are detailed in FIG. 5.

### Example 5—IgG ELISAs

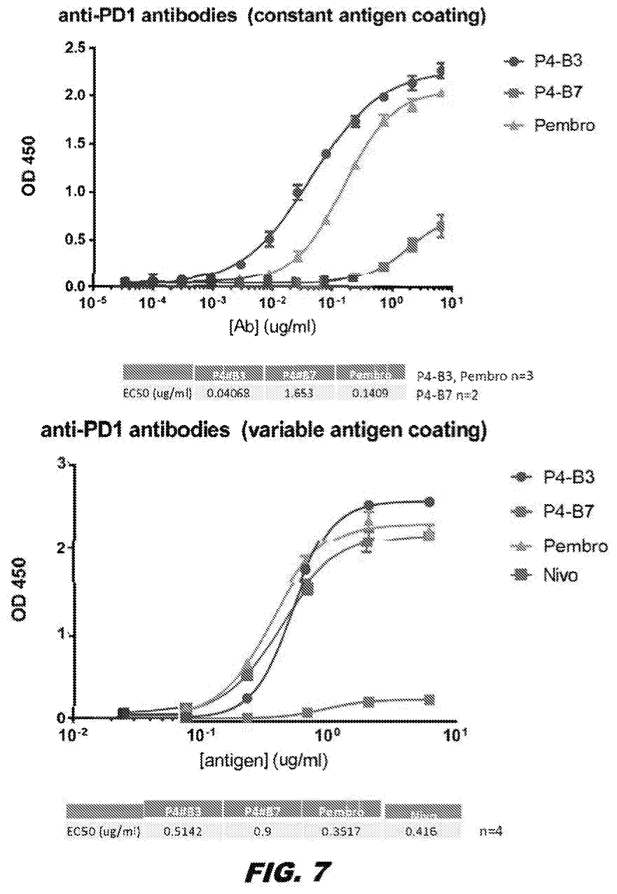

ELISA plates were coated with 1 ug/ml soluble PD1 for 2 hours at 37° C. The plates were then washed and blocked with 2% BSA/PBS at 37° C. for 1 hour. The blocking solution was removed and 3× serial dilutions of the antibodies were added to each well (100 ul) in 2% milk-PBST, starting with 6 ug/ml. The plates were then incubated at RT with gentle shaking, washed 6× with PBS-T, and the secondary anti-human Fc-HRP (1:150k, Bethyl) was added. The plates were again incubated at RT with gentle shaking for 1 hour before being washed 6× with PBS-T. TMB substrate was added and the plate was incubated at 30° C. for 10 min to accelerate the HRP reaction. The signal was then quenched with TMB stop solution and read at 450 nm. See TOP graph of FIG. 7.

The same protocol as described herein was carried for the BOTTOM graph of FIG. 7 except the plate was coated with 3× serial dilutions of the antigen, starting at 6 ug/ml. The antibody was then added at a constant concentration of 1 ug/ml to all wells.

### Example 6—PD1 FACS with anti PD1 IgGs

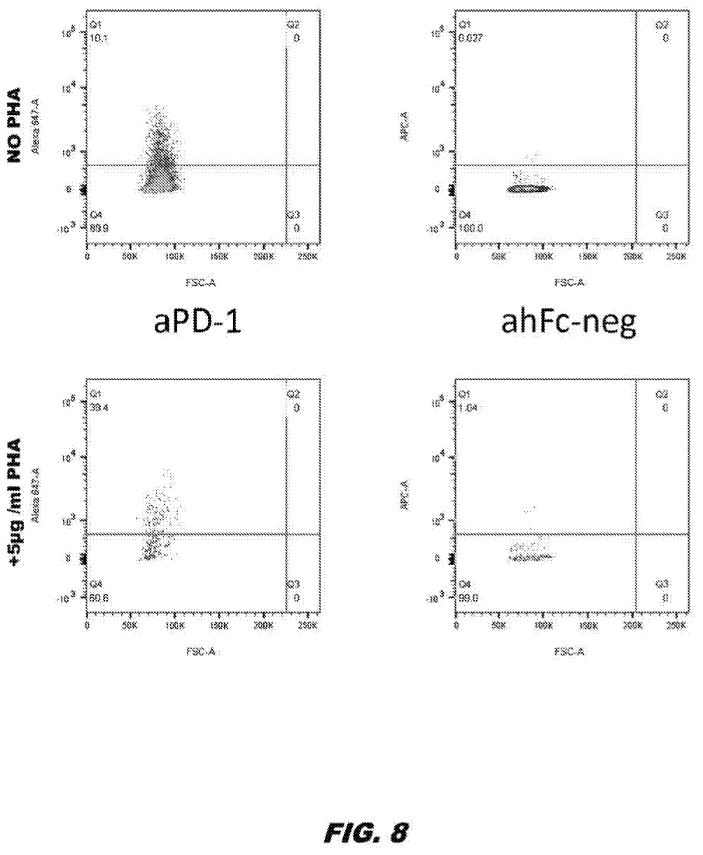

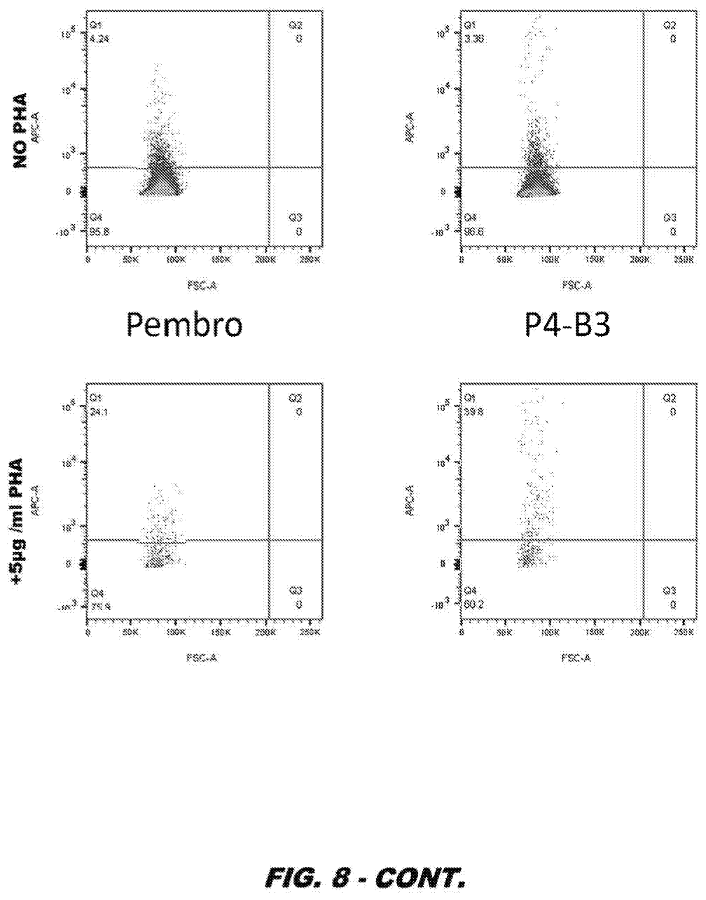

T cells were cultured for 48 hours with or without 5 ug/ml PHA in complete DMEM (293FT media). Pembrolizumab and the P4-B3 antibody were detected with Biolegend's anti human IgG Fc APC (Cat #409306). As shown in FIG. 8, P4-B3 PD-1 antibody displays a similar binding pattern to that of pembrolizumab and the control anti-PD1 antibody.

### Example 7—PD1-PDL1 Bioassay

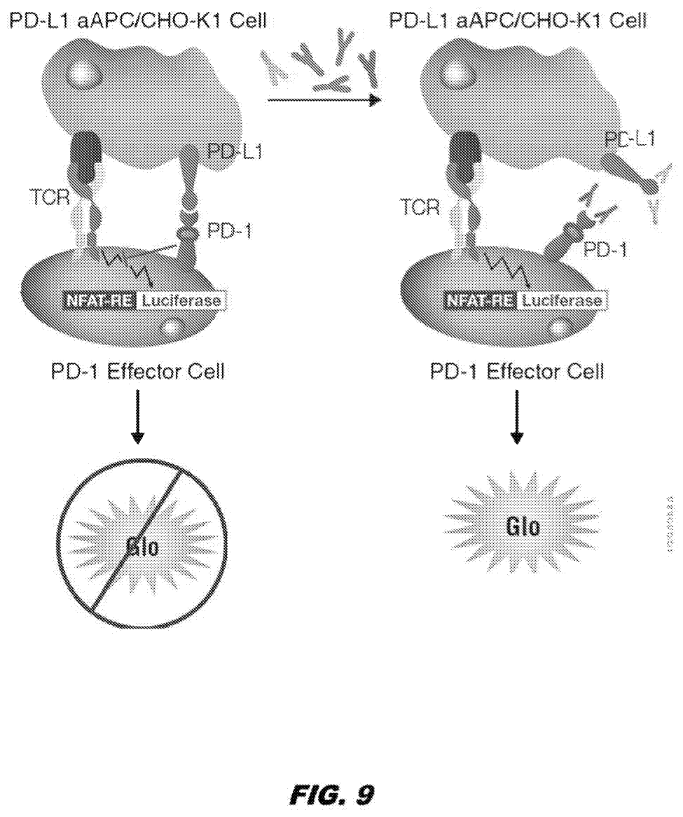

The Promega PD1-PDL1 bioassay (J1250) was carried out with a PD-1 antibody of the invention (P4-B3) and the commercial antibodies pembrolizumab and nivolumab (FIG. 9).

Constructs tested included: (a) IgG₁: WT monomer; (b) LALA: monomer, hexamer, and mutant 3; (c) sIgG4: monomer and hexamer; Control: mAb11 LALA monomer

All samples were done in triplicate except for mAb11.

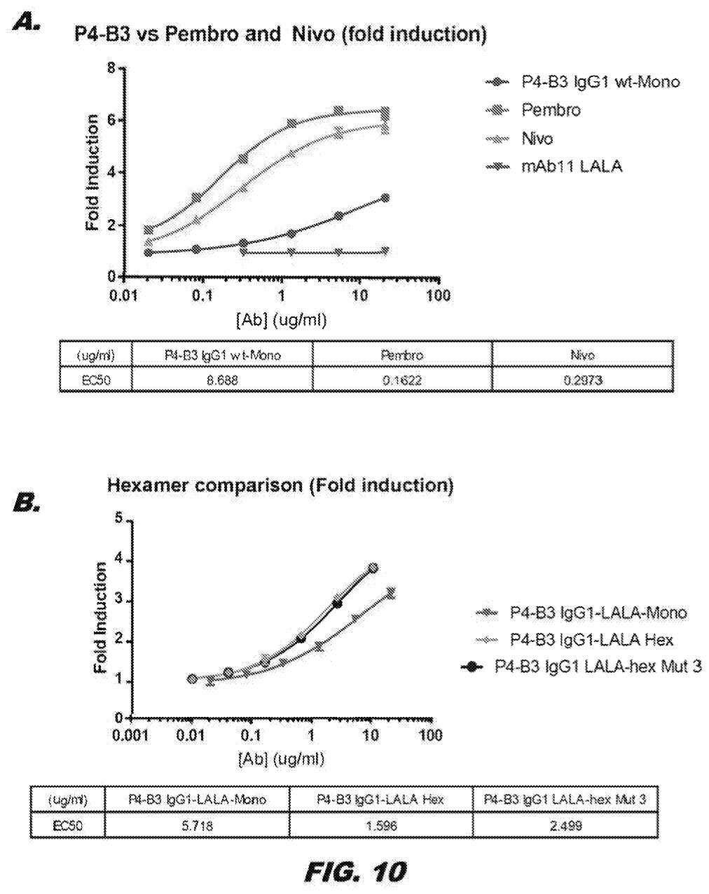

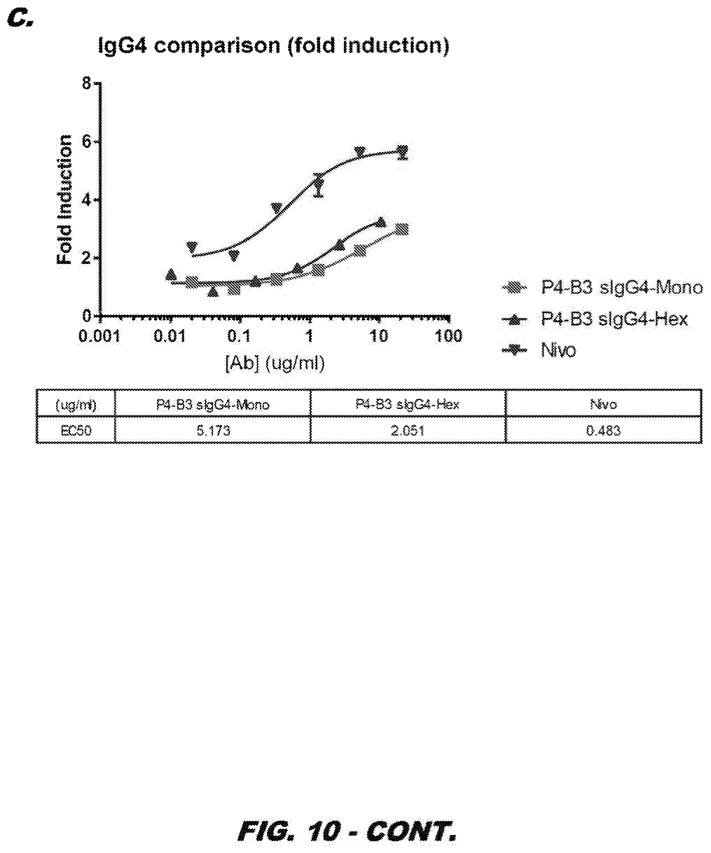

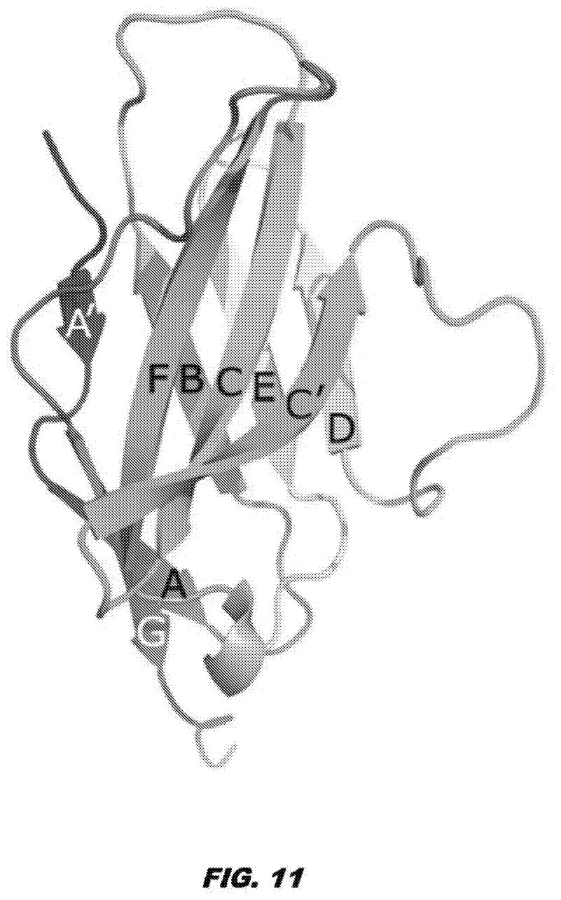

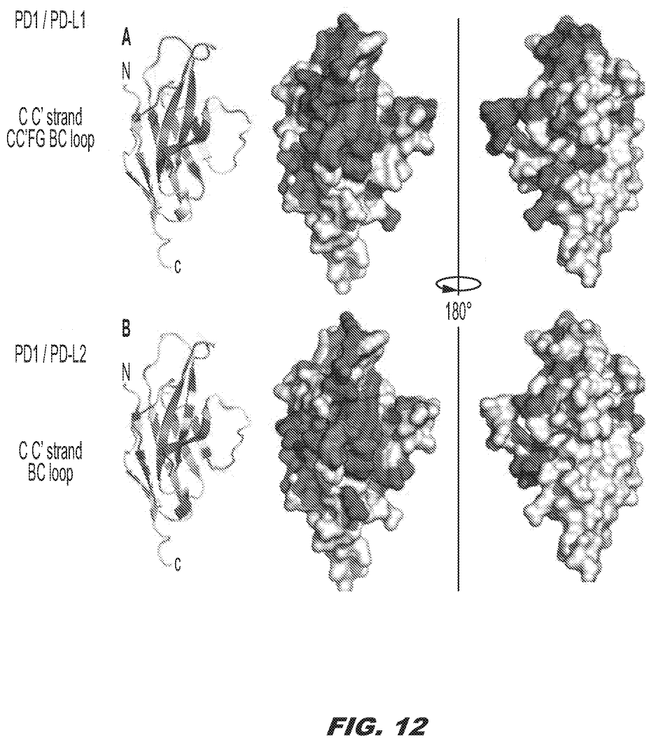

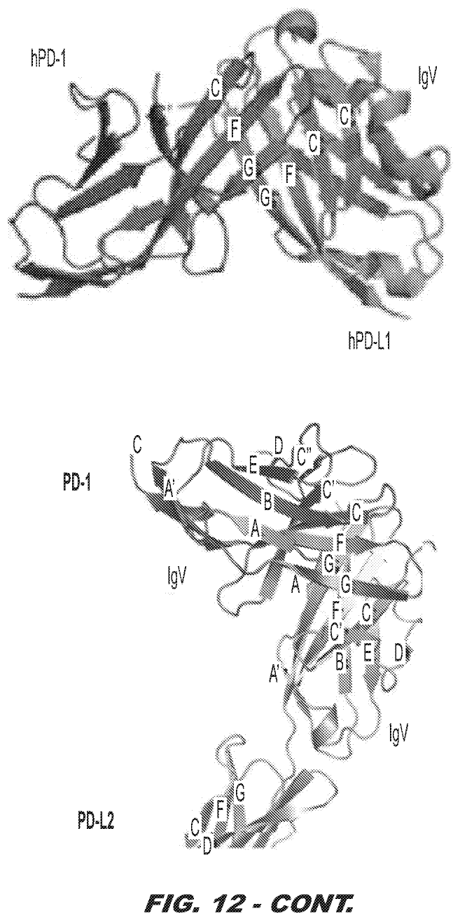

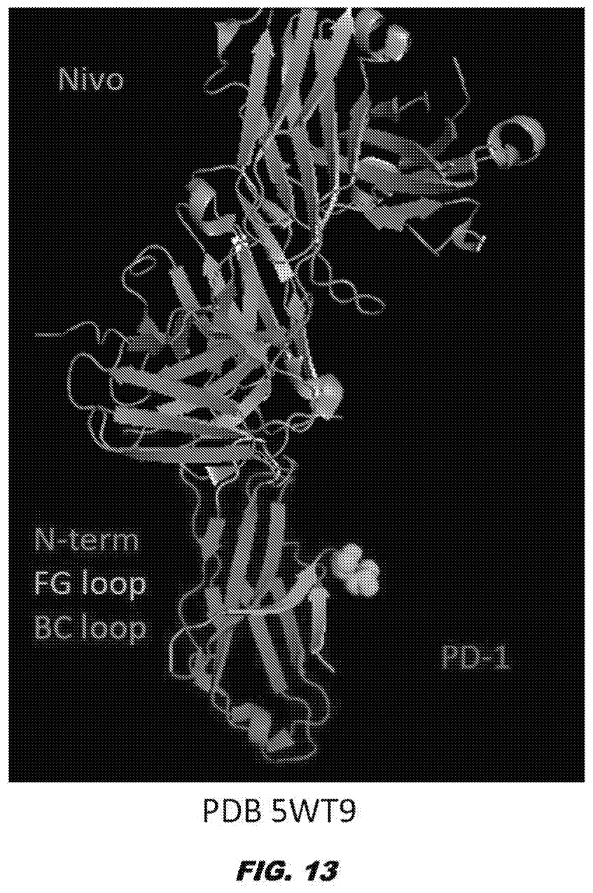

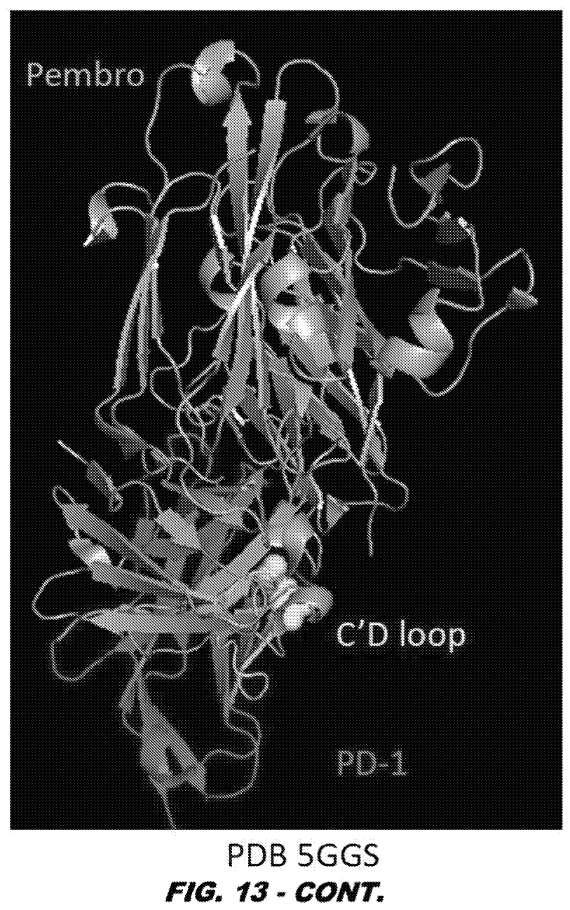

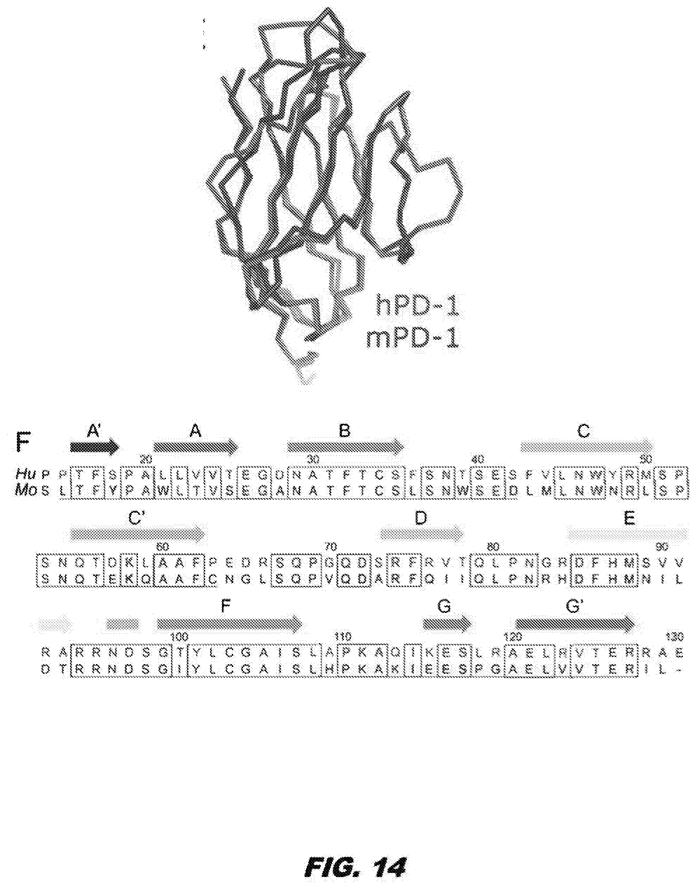

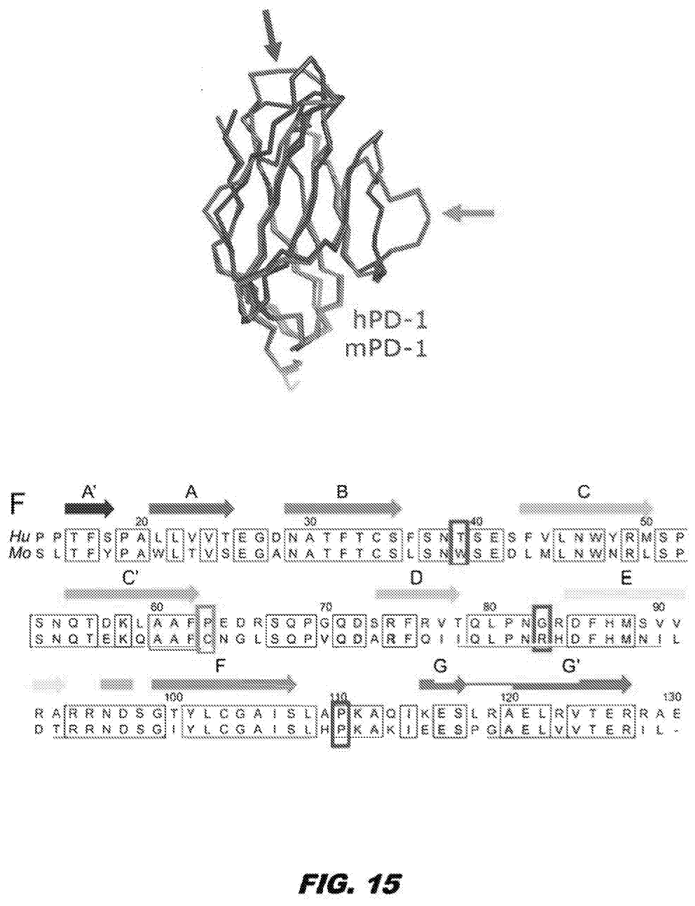

Fold induction: RLU stimulated/RLU unstimulated (no Ab) (FIG. 10).

### Example 8—Anti-PD-1 Cross Reactivity

Many anti-PD-1 antibodies are not able to cross react with mouse and human PD-1 (Pembro and Nivo are not cross reactive). See Fessas, Petros et al. “A molecular and preclinical comparison of the PD-1-targeted T-cell checkpoint inhibitors nivolumab and pembrolizumab” Seminars in oncology vol. 44,2 (2017): 136-140. See also, Tan J B L, Chen C, Chen K, Preclinical Characterization of GLS-010 (AB122): A Fully Human Clinical-Stage anti-PD-1 Antibody.” Poster, Arcus Biosciences; See, Burova, Elena et al. “Characterization of the Anti-PD-1 Antibody REGN2810 and Its Antitumor Activity in HumanPD-1 Knock-In Mice” Large Molecule Therapeutics, 2017. Further, see Li, Dong et al. “Epitope mapping reveals the binding mechanism of a functional antibody cross-reactive to both human and murine programmed death 1” mAbs vol. 9,4 (2017): 628-637.

The antibodies of the invention (e.g., P4-B3) is cross-reactive.

3E5 transiently transfected Expi293 cells were suspended in 100 ul MACS buffer and added to each well. 50 ul of each antibody dilution were then mixed with the cells and the plate was incubated at 4° C. for 30 min. Following incubation, the plate was washed 2× with MACS buffer and then incubated with 1 ul/well anti human Fc-APC (Biolegend #409306). The plate was incubated for 25 min at 4° C. and then washed 3× before running the samples.

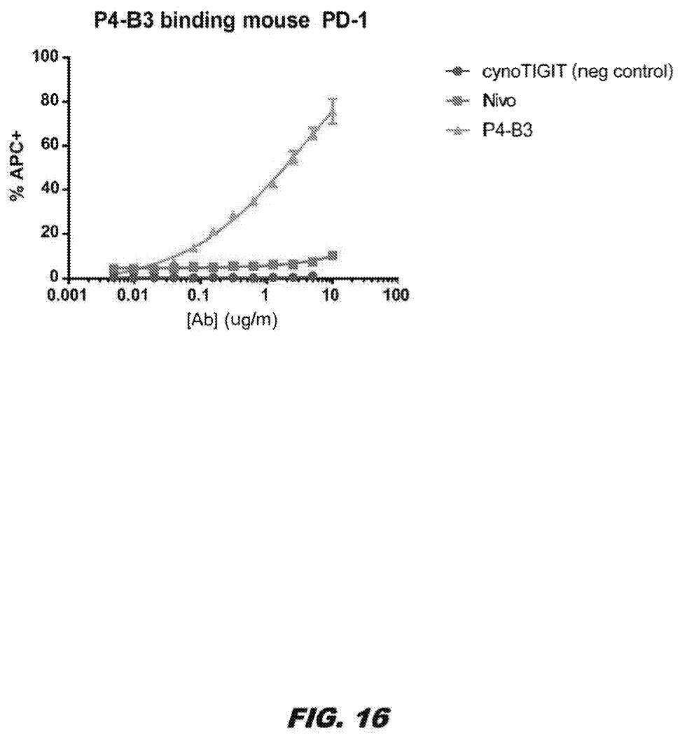

As shown in FIG. 16, P4-B3 has reasonable affinity to mouse PD-1, setting it apart from Pembro and Nivo.

### Example 9—Affinity Maturation

Generating Yeast Library.

First, cut and paste P4-B3 scFv from pFarber vector (phage display) into pCTCON2 vector (yeast display). Then make library according to two methods practiced in the art: (1) Digestion/ligation in bacteria and transform intact plasmid into yeast; and (2) Linearized vector+PCR fragment(s) for homologous recombination in yeast. The digest/ligation method (method (1) described herein) yielded a very low library size—low efficiency of ligation/bacterial transformation and very low efficiency transforming back into yeast. However, homologous recombination (method (2) described herein) yielded libraries with ˜10⁶-10⁷ mutants.

Error Prone Mutagenesis.

The Agilent GeneMorph II Random Mutagenesis Kit was used. The kit is designed to vary mutation rate based upon initial template DNA.

| | Mutation | |
|---|---|---|
| Mutation | frequency | Initial target |
| rate | (mutations/kb) | amount (ng) |
| Low | 0-4.5 | 500-1000 |
| Medium | 4.5-9 | 100-500 |
| High | 9-16 | 0.1-100 |

External Primers (˜50-60 bp Overlap with pCTCON2 Vector):

(a) pCTCON2-HR-Fwd:
GAGGAGGCTCTGGTGGAGGCGGTAGCGGAGGCGGAGGGTCGGCTAGCTG
GGCCCAGCCGG
(b) pCTCON2-HR-Rev:
ACACTGTTGTTATCAGATCTCGAGCTATTACAAGTCCTCTTCAGAAATA
AGCTTTTGTTC

Internal Primers (45 bp Overlap with Heavy or Light Chain Fragment):

(a) G4S-Fwd:
GGTGGCGGCGGTTCCGGAGGTGGTGGTTCTGGCGGTGGTGGCAGC
(b) G4S-Rev:
GCTGCCACCACCGCCAGAACCACCACCTCCGGAACCGCCGCCACC

Error Prone Mutagenesis Strategies.

(a) PCR entire scFv fragment with external primers. This strategy allows for mutations in the linker region (which is not desired). (b) PCR Heavy chain and light chain separately using external primers and G4S primers, use the G4S linker as a third overlap point for 3 piece homologous recombination. This strategy protects the linker from mutations, however requires a 3 piece homologous recombination which might be less efficient than 2 pieces.

Both techniques were used with varying amount of template DNA. Template for entire scFv PCR is the pCTCON4 vector with P4-B3 cloned in (˜ 1/10 template is target seq). Template for heavy/light chain separate PCR is a P4-B3 PCR fragment (˜½ of template is target seq).

Template used—For PCR of entire scFv: 4 ug, 2 ug, 1 ug, 0.5 ug; For PCR of heavy/light chains separately: 450 ng, 50 ng (2 reactions each)

*PCR was run for 33 cycles to increase DNA yield

Library Generation.

Followed protocol described in Benatuil et al, “An improved yeast transformation method for the generation of very large human antibody libraries,” Protein Eng Des Sel. 2010 April; 23(4):155-9.

General protocol: EBY100 yeast cells were inoculated in 100 ml YPD media at OD600=0.3 and grown for ˜5-6 hours at 30° C. until OD600=1.6. Cells were collected by centrifugation and washed 2× with 50 ml cold ddH₂O, and once with 50 ml cold electroporation buffer (1 M sorbitol/1 mM CaCl₂)). Cells were then conditioned by shaking at 30° C. for 30 min in 20 ml 0.1 M LiAc/10 mM DTT. Cells were harvested and washed with 50 ml cold electroporation buffer. After pelleting, the cells were resuspended to a final volume of 1 ml, which is good for 2 transformations.

Whole scFv PCR: yielded 4.8 ug insert so mixed with 4 ug linearized vector (NcoI/BamHI)

H/L chain PCR: yielded 4.1 ug HC and 3.5 ug LC, decrease to 3 ug linearized vector (NcoI/BamHI)

Vector and desired fragments were mixed then EtOH precipitated to decrease volume (less than 50 ul). 400 ul of electrocompetent yeast cells were transformed using Biorad, at 2.5 kV, and 25 uF. Cells recovered in 1:1 YPD:1 M sorbitol for 1 hour before spinning cells down, washing with SDCAA, and resuspending in 250 ml SDCAA for each transformation.

Titers: (a) whole scFv library: ˜5.2E6 members; (b) H/L chain separately: ˜5.8E6 members.

After 2 passages, the colonies were plated out for sequencing (96 colonies per library). The whole scFv library: 56/96 (58.33%) had at least one mutation. The H/L chain separate library: 42/96 (43.75%) had at least one mutation,

Effective library size: (a) whole scFv library: ˜2.9E6 members; (b) H/L chain separately: ˜2.1E6 members

Library Sorting Strategy.

Two staining methods were used: (1) Standard staining looking for improved binding (shift to the upper right quadrant during FACS analysis); and (2) Kinetic strategy looking for improved off rate.

For kinetic staining, the library is stained with labeled antigen at a concentration 10 times greater than the Kd, washed and then incubated in an increased volume and with an unlabeled antigen at concentrations 100× greater than the Kd. Incubating sample in larger volume makes it so that any antigen that dissociates is unable to rebind with the yeast. Additionally, adding a higher concentration of unlabeled antigen means any labeled antigen that comes off will be replaced with unlabeled antigen.

For kinetic staining, the staining time depends on the time constant (τ).

τ=(kₒₙ[Ag]₀+k_{off})⁻¹

Where kon=on rate (M\hat{ }-1 s\hat{ }-1); koff=off rate (s\hat{ }-1); and [Ag]0=initial antigen concentration (M)

From octet measurements, P4-B3 scfv has kon=6.85E4, koff=6.45E-5, Kd=9.4E10.

Binding at 95% of equilibrium binding by 3c and 99% at 5τ

The staining protocols were carried out according to Cherf and Cochran, “Applications of Yeast Surface Display for Protein Engineering,” Methods Mol Biol. 2015; 1319:155-75.

Briefly, high-affinity protein variants were isolated from a yeast-displayed library by FACS. Following transformation of yeast cells with a gene library and induction of surface expression, two main strategies are used to differentially label the displayed library prior to screening: 1) an equilibrium binding strategy where the library is incubated with a ligand concentration 5-10-times greater than the expected K_{D} value of the highest affinity variant, resulting in near saturation of tight binding variants and partial labeling of weaker affinity variants at equilibrium, and 2) a kinetic binding strategy where the library is incubated with ligand as described for the equilibrium binding strategy, but unbound ligand is removed by washing and the library is then either incubated with a 100-fold excess of unlabeled ligand, or incubated in a sufficiently large volume of buffer to prevent rebinding of dissociated ligand.

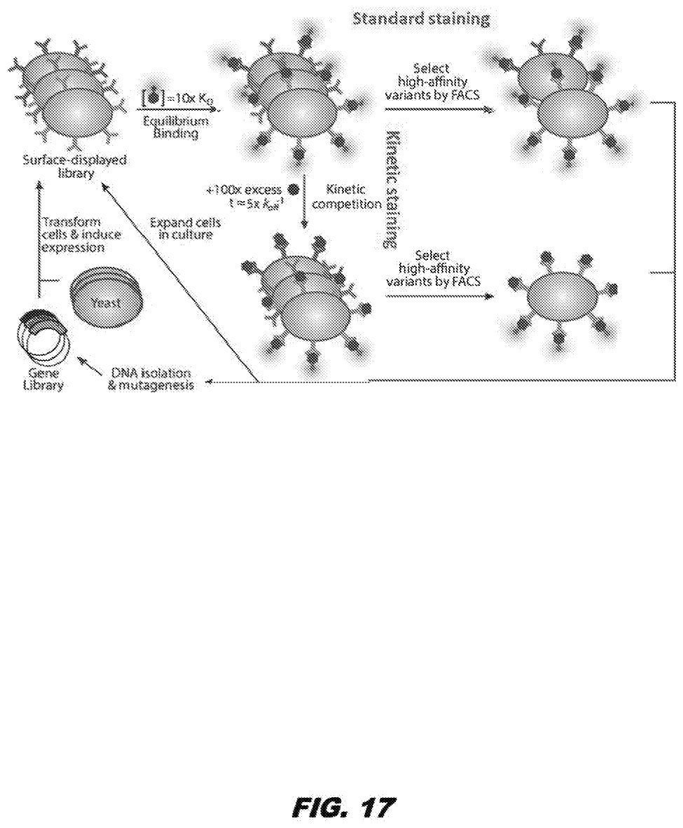

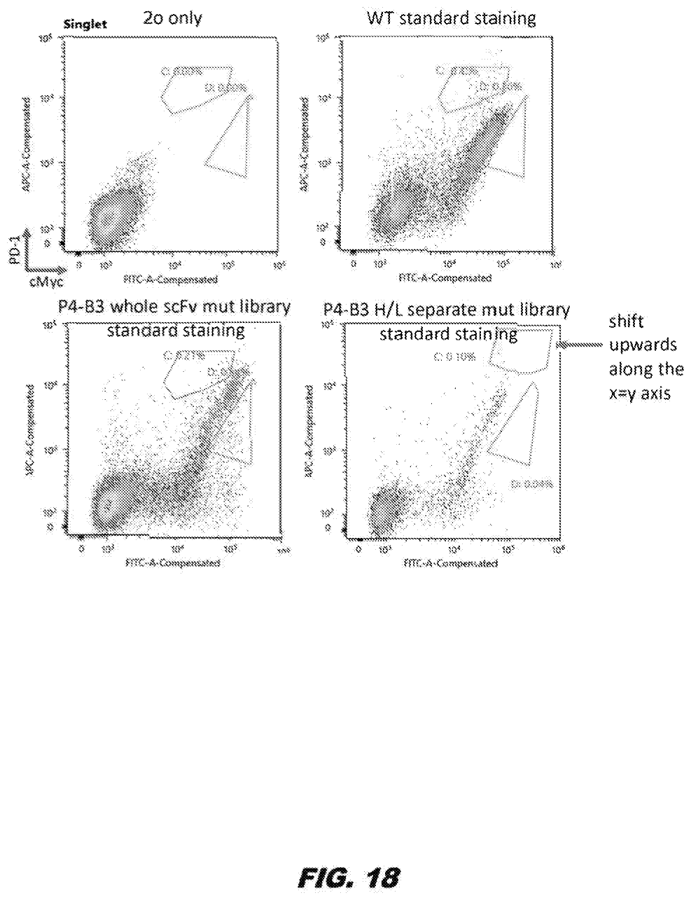

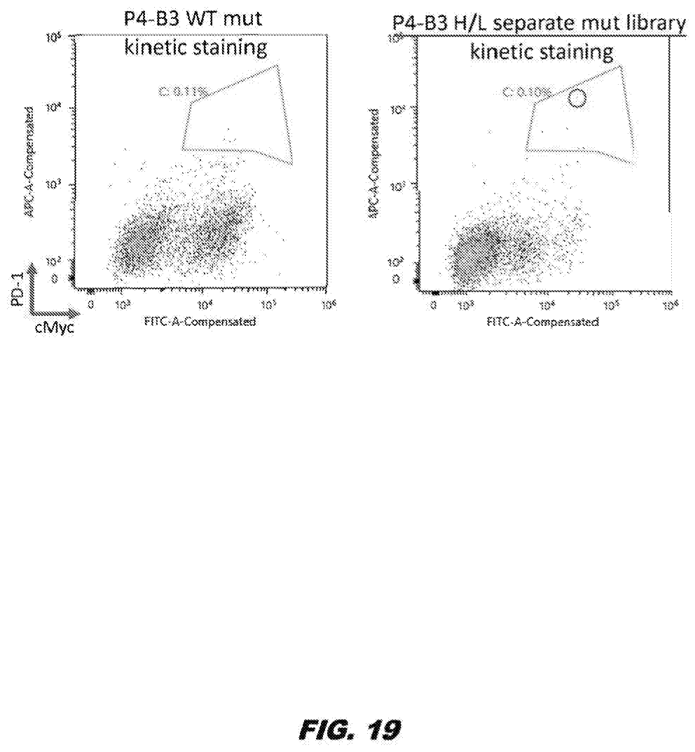

During this second incubation step, the excess unlabeled ligand or large incubation volume prevents dissociated labeled ligands from rebinding. Proteins are thus differentiated based on their dissociation rate constants (koff), with variants having the slowest koff retaining the largest percentage of pre-bound labeled ligand. Addition of a fluorescently-labeled anti-epitope tag antibody permits normalization of yeast surface expression levels with binding, allowing the highest affinity variants to be isolated by FACS. Sorted pools of yeast clones can be expanded in culture for either analysis or a subsequent round of sorting, or DNA from these clones can be isolated, subjected to mutagenesis, and used to transform a new batch of yeast for further directed protein evolution. Components of the yeast display platform, including Aga1p, Aga2p, HA and c-myc epitope tags, and detection antibodies depicted in FIG. 17, are omitted for clarity.

Library Sorting.

Library was sorted on Sony SH800 and ˜1000 clones were collected per sample. Samples were sorted for clones with increased and decreased binding (important residues are being mapped out). The sorted cells were plated out and only a couple dozen grew, of which all were sequenced. Focused on the H/L chain separate library with the standard and kinetic staining.

| Clone | mutation spot |
|---|---|
| HL kinetic 1 | CDRH1 |
| HL-2 | FW3/CDRL3 |
| HL-7 | CDRL2 |
| HL-10 | CDRL2 |
| HL-14 | CDRH3 |

Sorted cells were plated on SDCAA plates and incubated 30° C. for 3 days. The colonies were then picked, grown in fresh SDCAA media, and sequenced to identify important mutations. Unique clones from sequencing were then inoculated in fresh SGCAA (induction via galactose) and after 36 hours the samples were stained to generate binding curves.

EBY100 yeast cultures were induced for 1.5 days at 30° C. 1E6 cells was spun down and placed into wells with varying dilutions of antigen in PBS. The plate was incubated at RT for 2 hours with shaking. The plate was washed with PBS and 0.1 ug/ml streptavidin-APC (biolegend) was added to each well. The plate was incubated at RT for 25 min with shaking and then washed and read on the FACS caliber.

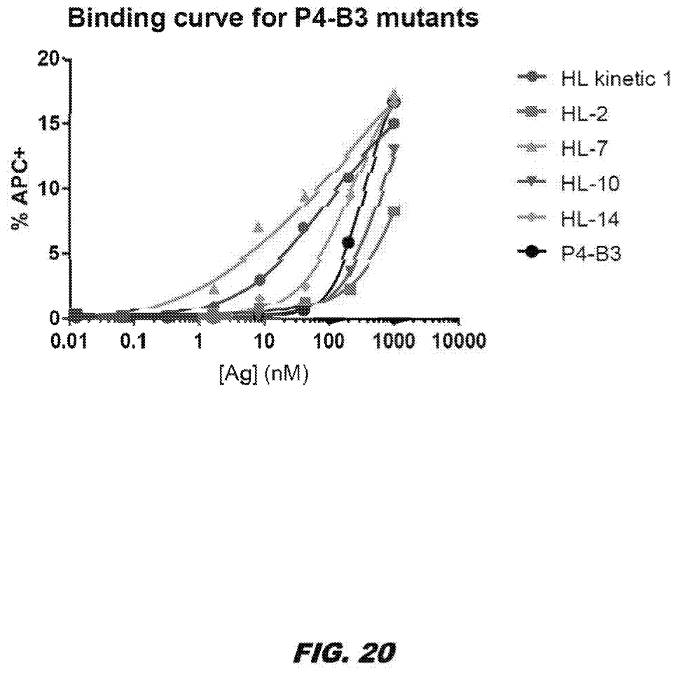

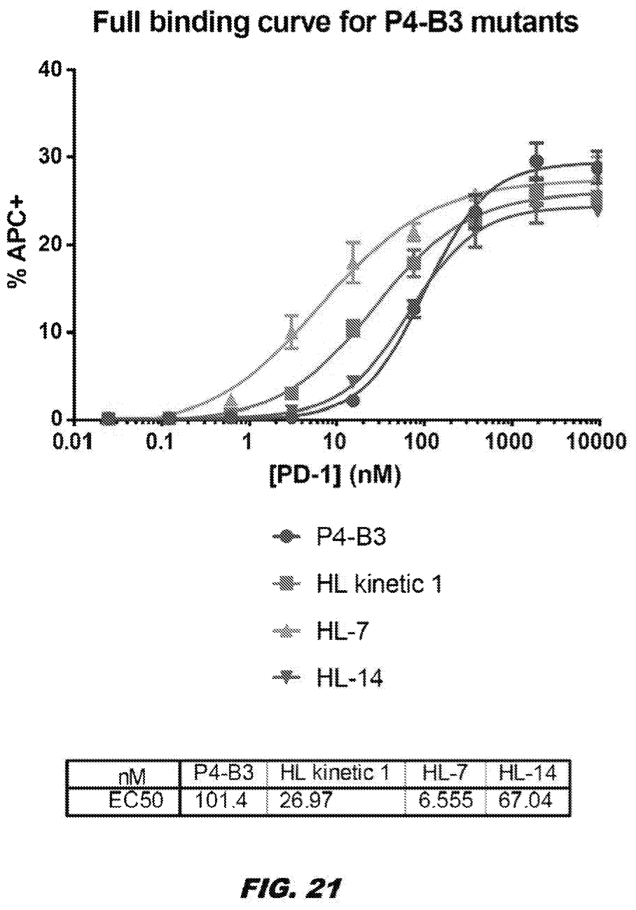

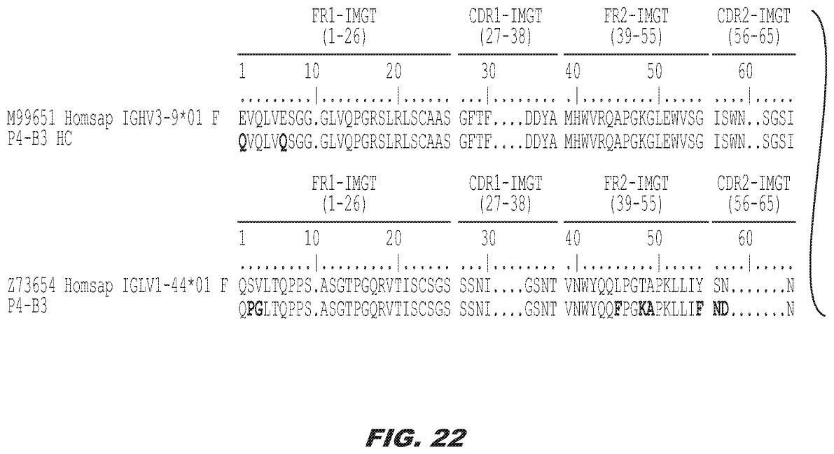

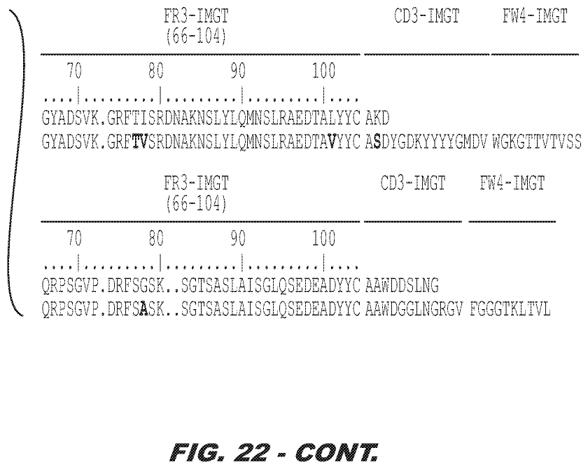

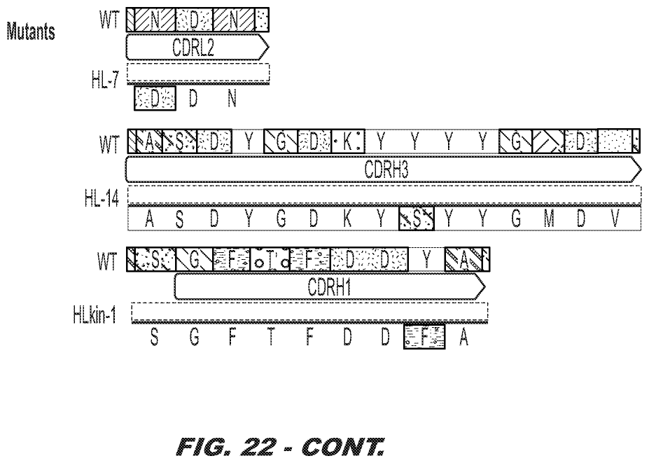

Clones 2, 7, 10, 14 came from the random mutagenesis library of P4-B3 (anti-PD1), sorted for higher binding (shifted up the y=x axis). HL clones were generated by error prone of the H and L chains separately before being recombined via homologous recombination via the linker sequence. HL kinetic 1 came from a kinetic staining approach, where the library was incubated with 10×Kd of labeled antigen followed by a long incubation with 100 fold excess unlabeled antigen in 10× the original staining volume. P4-B3 wt was not positive at this stage, however there were a few clones in the library that popped up (See FIG. 20). Experiment was repeated with appropriate concentrations and only used the clones that shifted the curve to the left (See FIG. 21).

Other Clones Identified but yet to be characterized.

| | |
|---|---|
| scFv pos 1 | none |
| scFv pos 2 | stop codon |
| scFv pos 3 | CDRH1, FWL3, linker |
| scFv pos 6 | CDRL1, FWL3 |
| scFv neg 1 | CDRH2 |
| scFv neg 2 | FWH3 |
| scFv neg 3 | ins in CDRL3, mut in CDRH3 |
| scFv neg 4 | CDRH3/CDRL3 |
| scFv neg 6 | FWL3 |

scFv pos are clones that demonstrated some increased binding, mostly by expressing lower amounts of cMyc but binding higher amounts of PD-1 (none shifted up on the x=y axis).

scFv neg are clones that demonstrated decreased binding compared to WT.

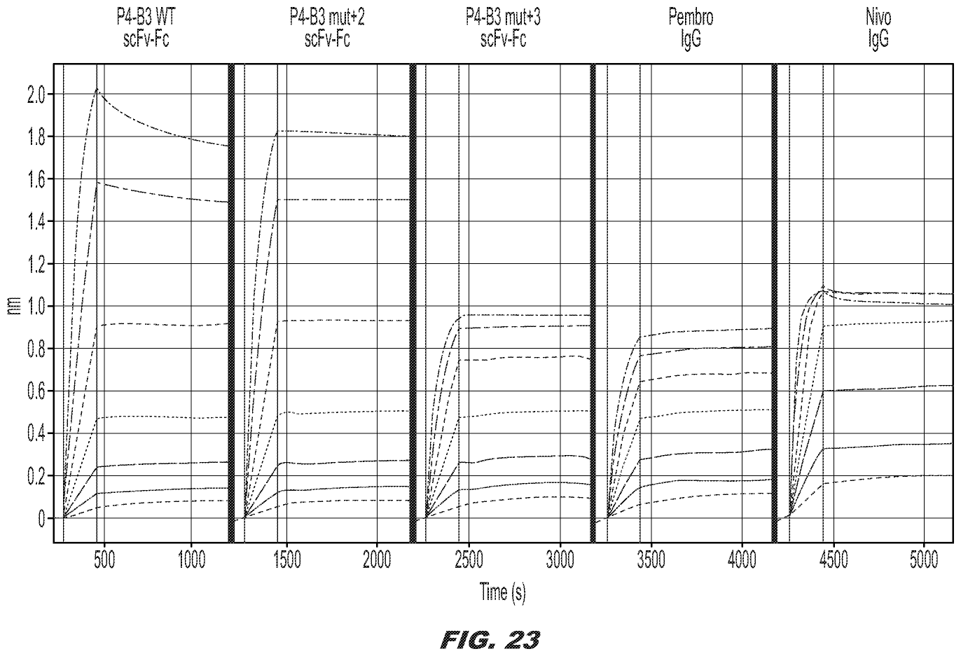

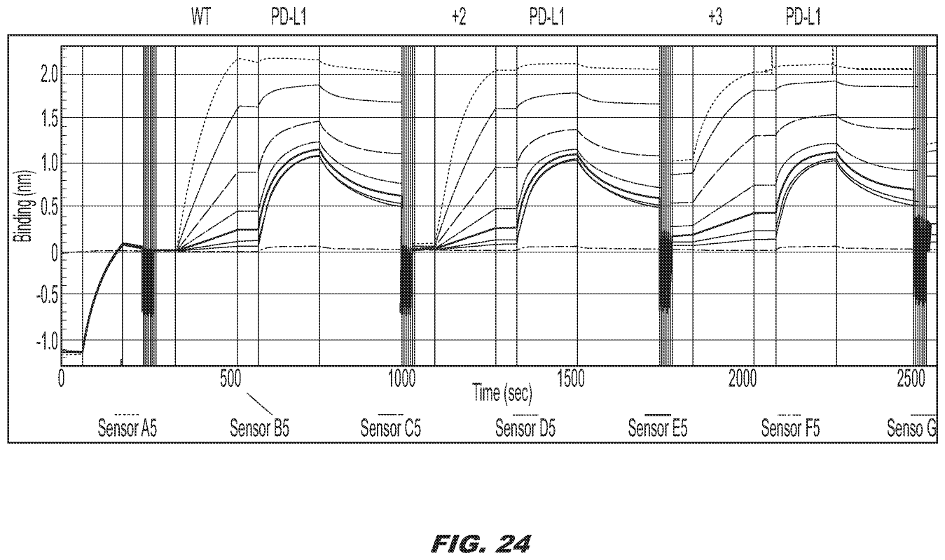

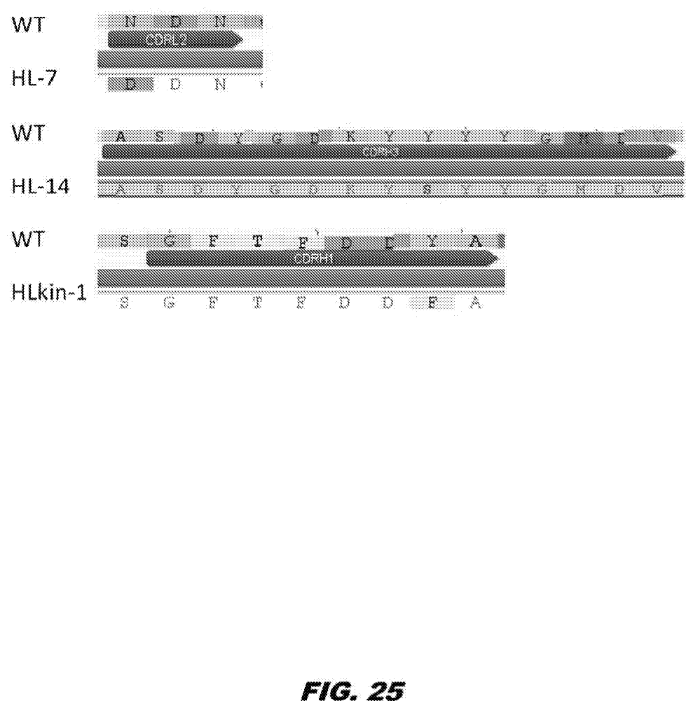

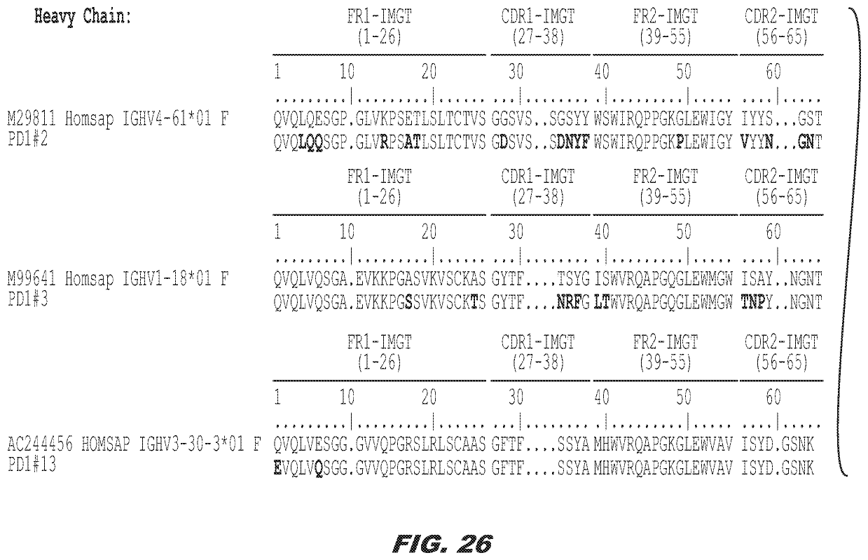

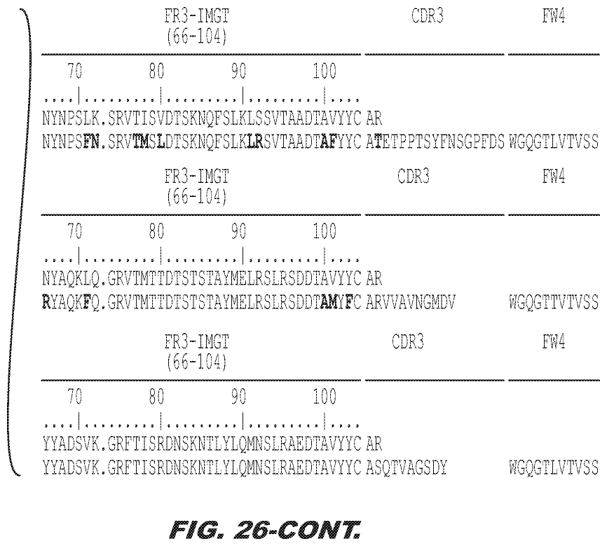

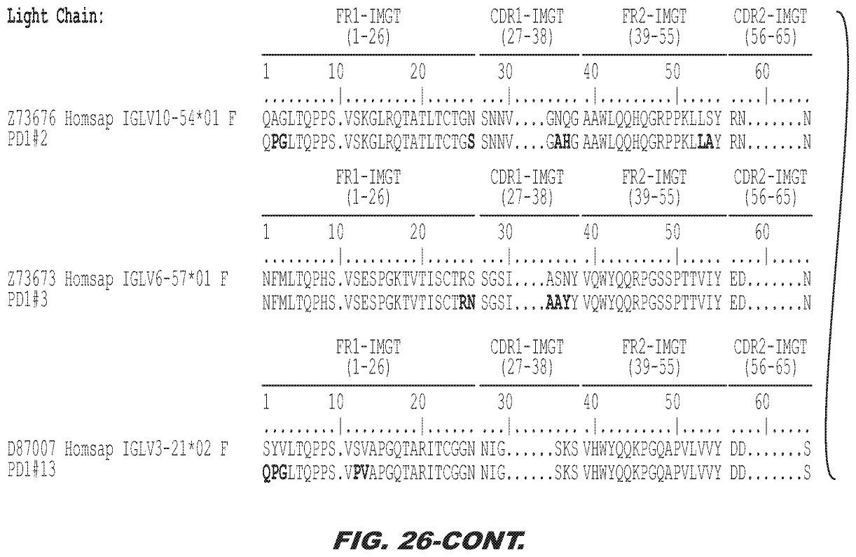

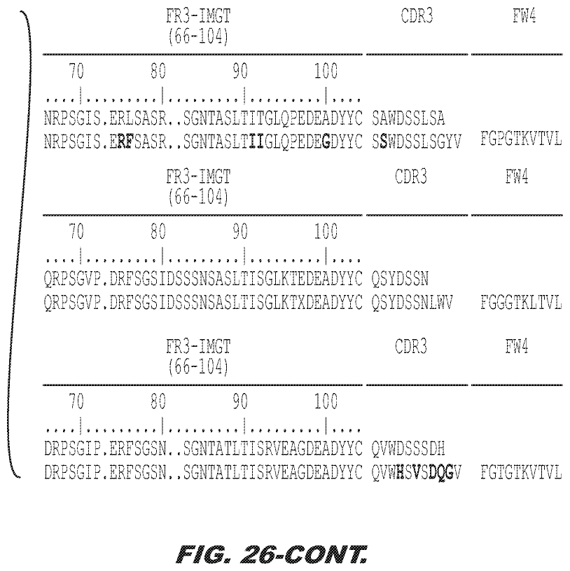

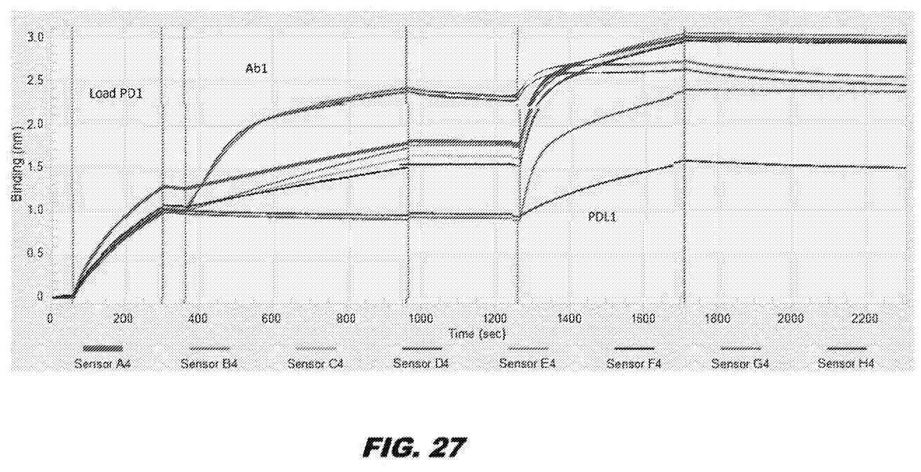

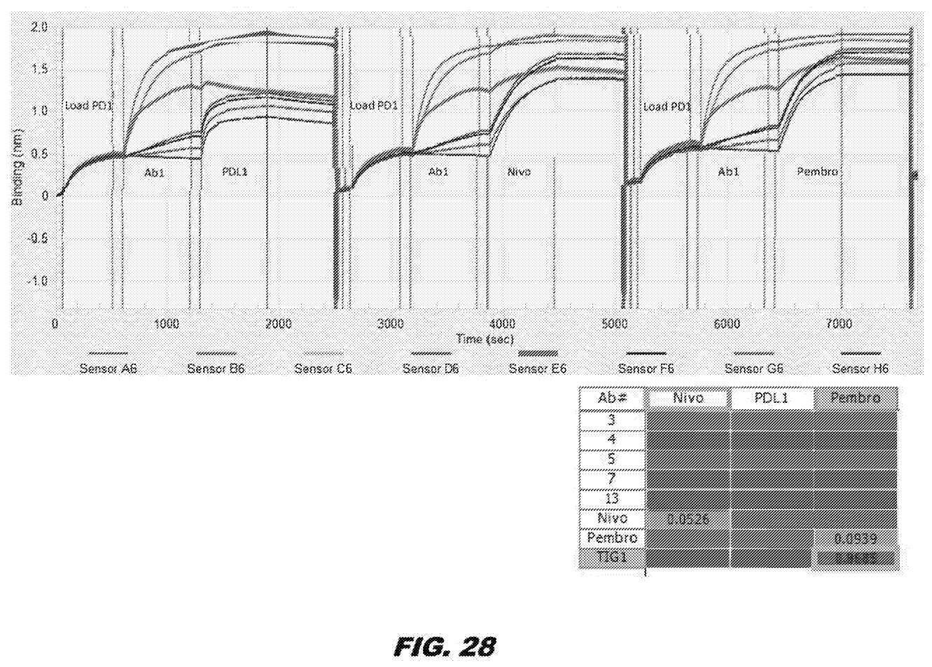

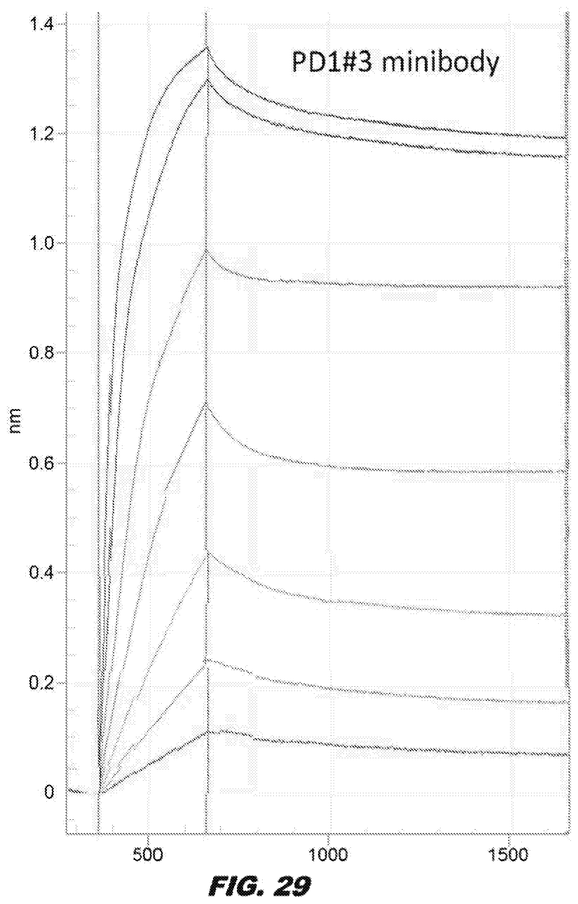

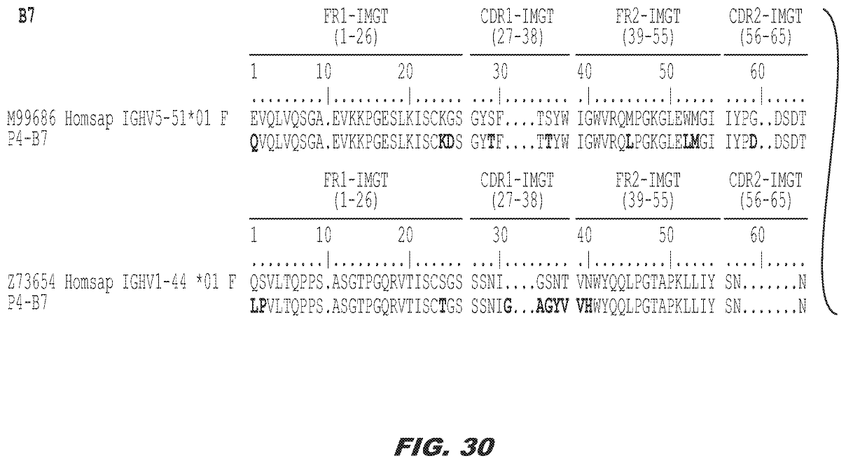

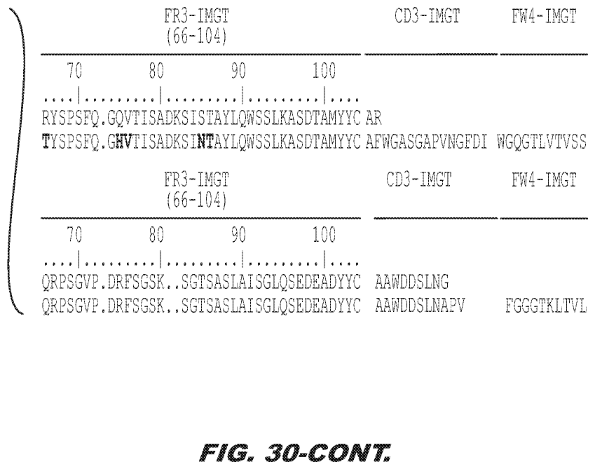

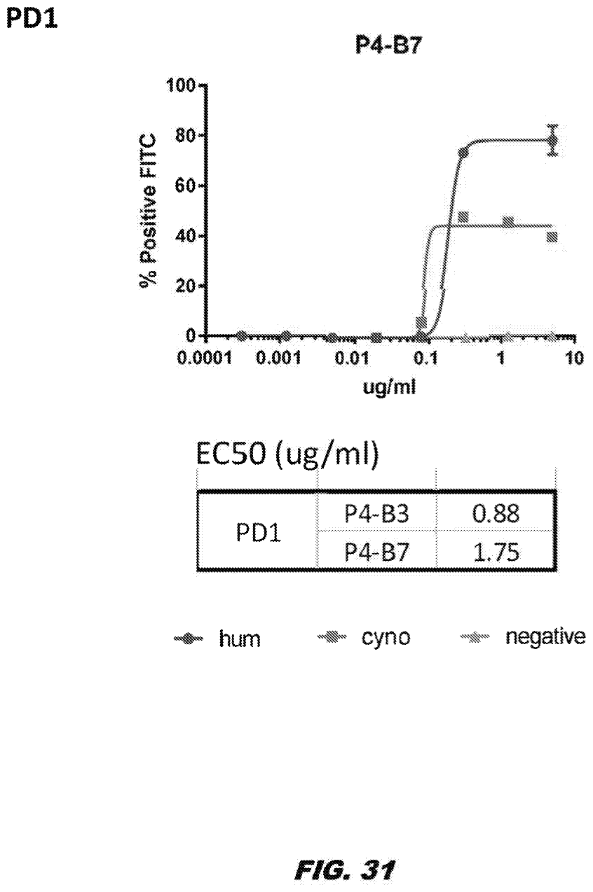

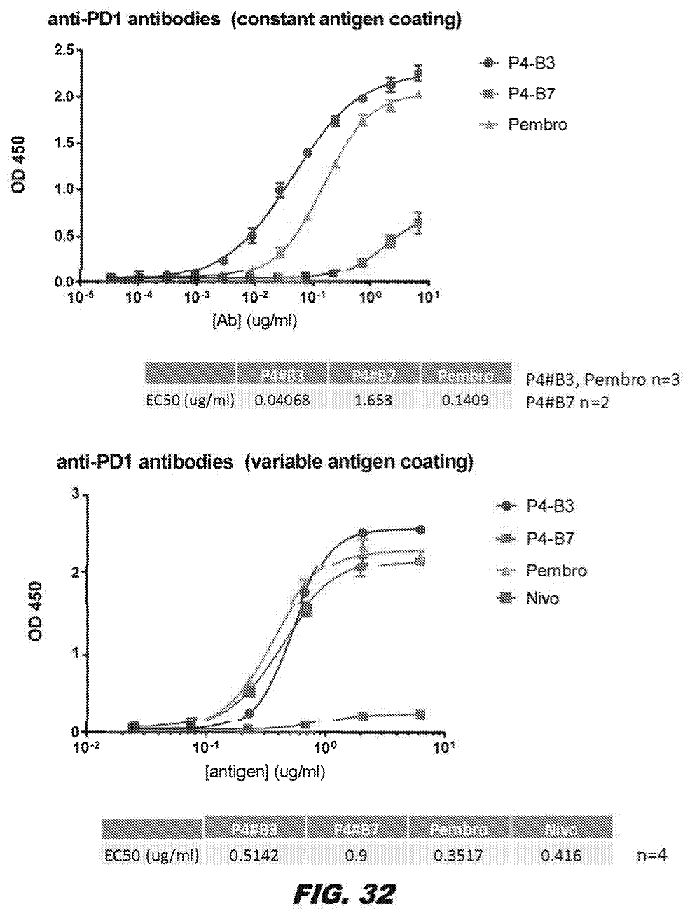

In addition to cloning HLkin-1, HL-7, HL-14 into minibody vector, made double (Mut+2: HLkin-1+HL-7) and triple (Mut+3: HLkin-1+HL-7+HL-14) combined mutant to see if an additive effect is observed (See FIG. 23 and FIG. 24).

For example, the following KDs have been measured:

PD1 #3˜1E-10 M

P4-B3 WT˜1E-9 M

Mut+2 (HLkin-1+HL-7)˜3E-11 M

Mut+3 (HLkin-1+HL-7+HL-14)˜3E-12 M

HLkin-1˜6E-11 M

### Example 10—PD1 Bioassay with IgG

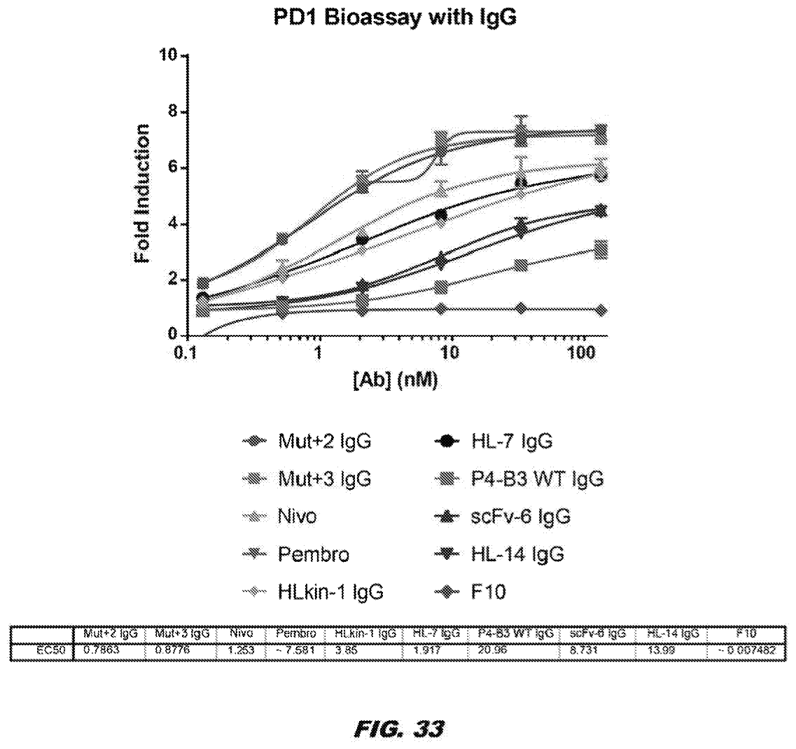

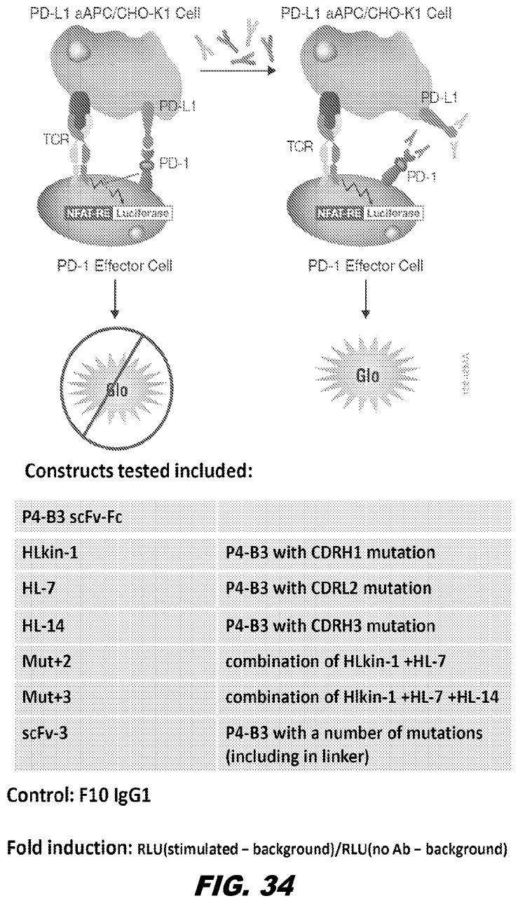

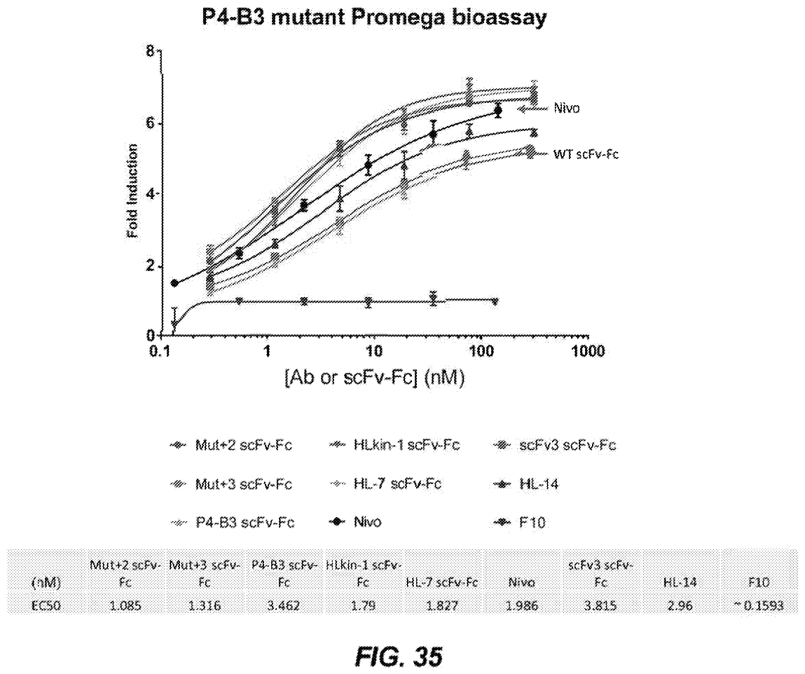

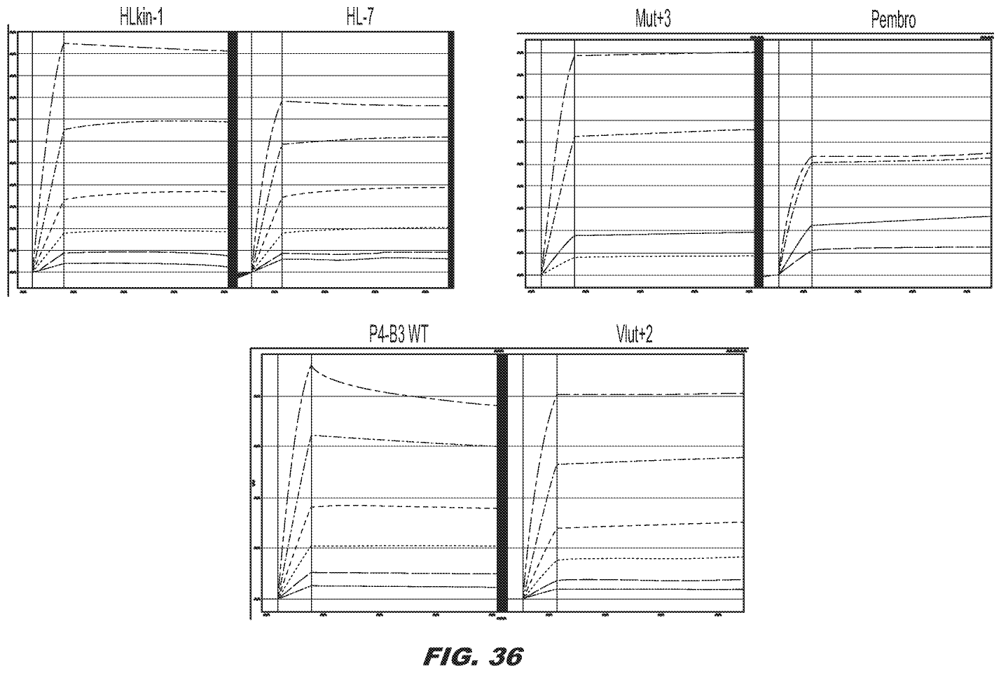

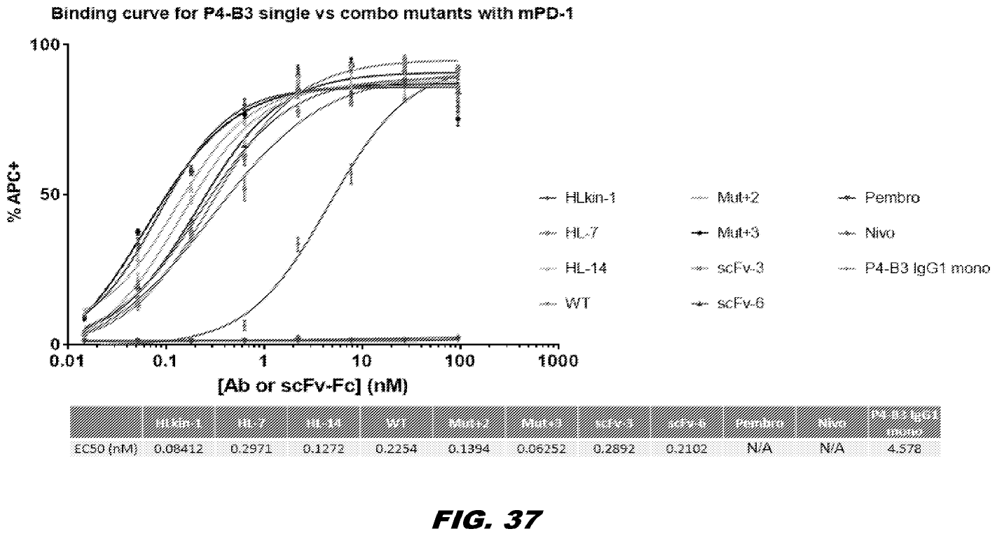

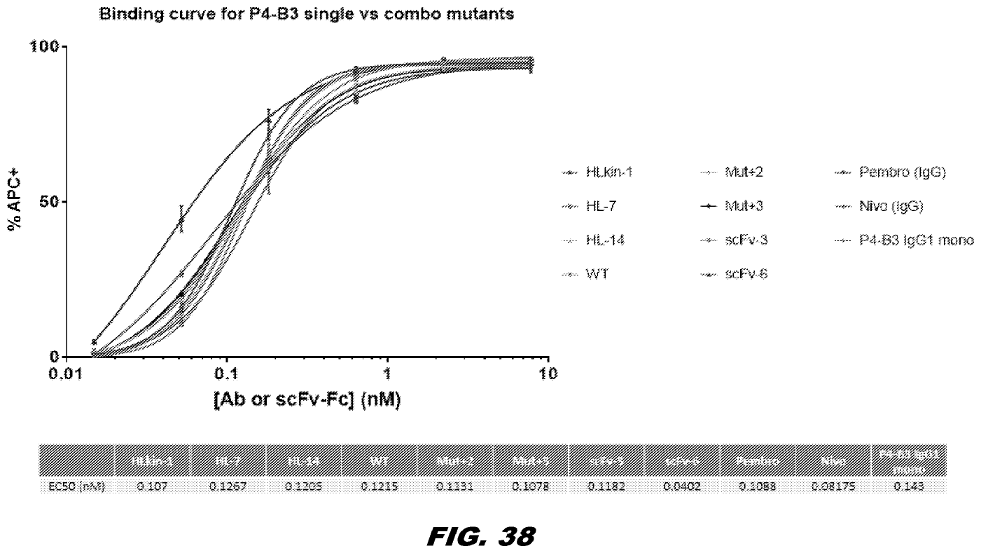

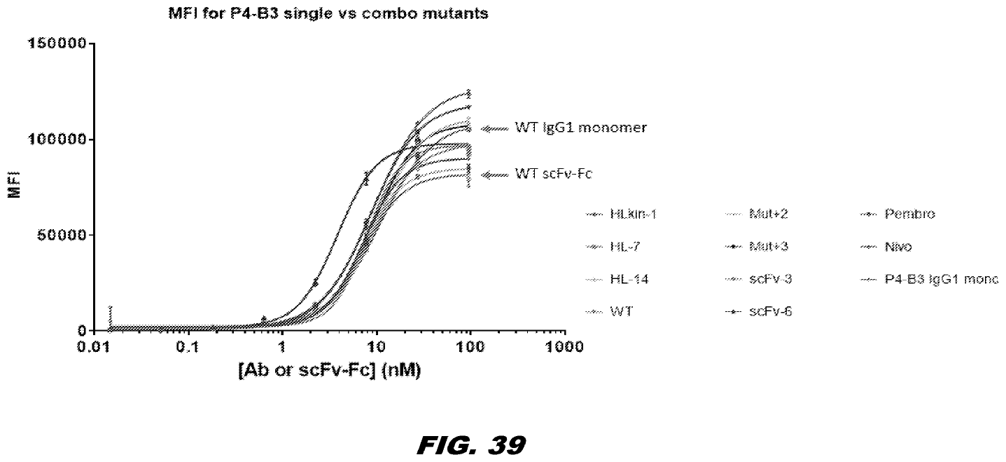

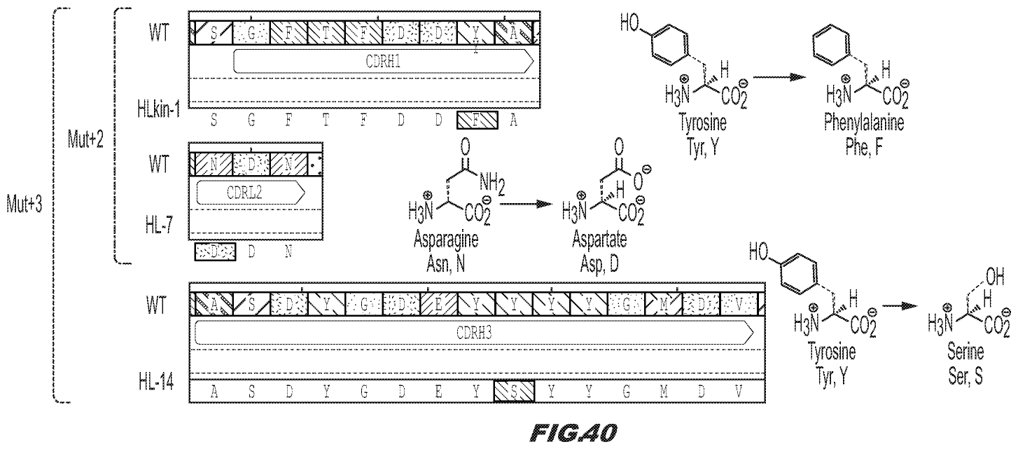

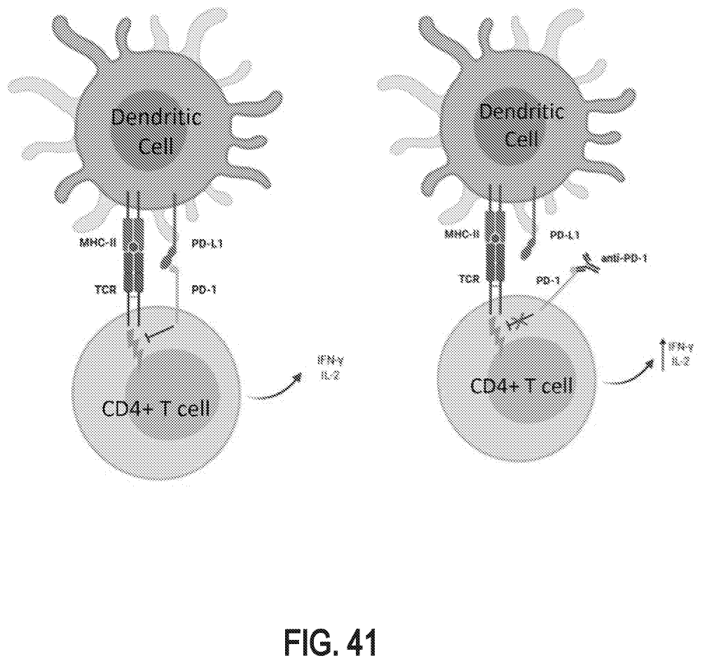

The Promega PD1-PDL1 bioassay (J1250) was carried out with PD-1 antibodies of the invention (e.g., P4-B3 and mutants described herein) and the commercial antibodies pembrolizumab and nivolumab (FIG. 33).

Nivo (green triangles) reaches a fold induction of about 5-6, which is similar to previous experiment. In the scFv-Fc experiment, Mut+2, Mut+3, HLkin-1, and HL-7 all showed an increased induction compared to Nivo. When converted to IgG, the combo mutants (Mut+2/Mut+3) continue to perform better than Nivo and at comparable levels to Pembro while the single (HLkin-1/HL-7) show slightly decreased activity. The original P4-B3 IgG is significantly below that of all the antibodies. Clone scFv-6 is a double mutant that came out of the yeast library and it has two light chain mutations. As seen, it is an improvement on P4-B3 WT but significantly worse than the commercial and other mutant antibodies.

### Example 11—Mixed Lymphocyte Reaction (MLR) Protocol

CD14+ monocytes were isolated using Miltenyi CD14+ microbeads. The cells were cultured in Miltenyi Mo-DC media (pre-prepared media with GM-CSF+IL4). The cells were cultured for 5 days, then the following was added: TNF-α (1000 U/ml), IL-1β (5 ng/ml), IL-6 (10 ng/ml) and prostaglandin E2 (PGE2) (1 μM) and the cells were cultured for 2 days to mature DC. T cells were isolated the day of the MLR experiment (CD4+ negative selection kit StemCell). 100,000 T cells and 10,000 MoDC cells were used per well for MLR. Antibodies were added at various concentrations and the cultures were incubated for 5 days.

Supernatant was saved for ELISA screening (e.g., IL2 and IFNγ). Cells were stained with CD4-FITC, PD1-PE, LAG3-BV421, TIM3-APC Cy7 for FACS analysis.

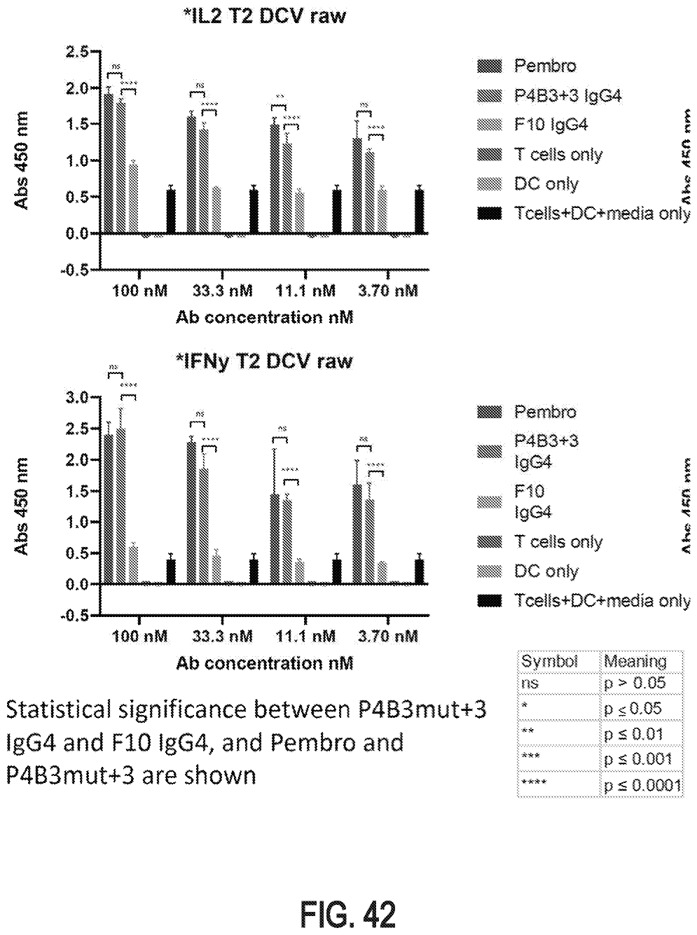

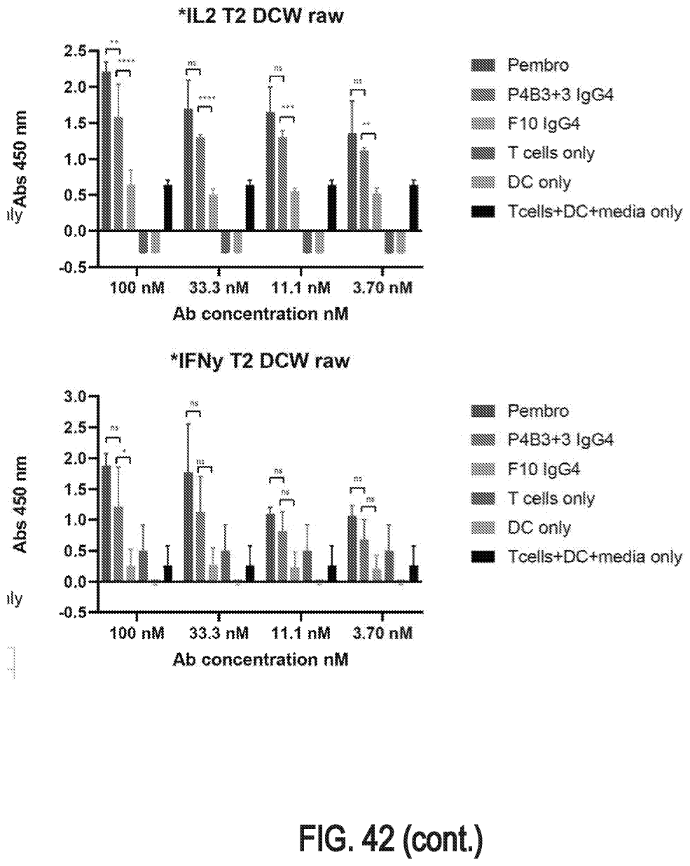

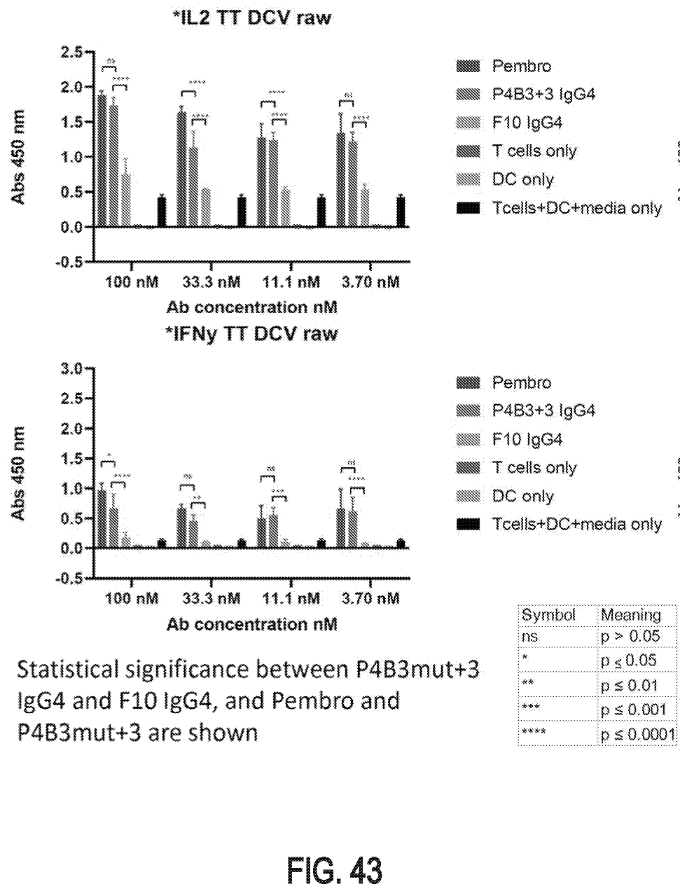

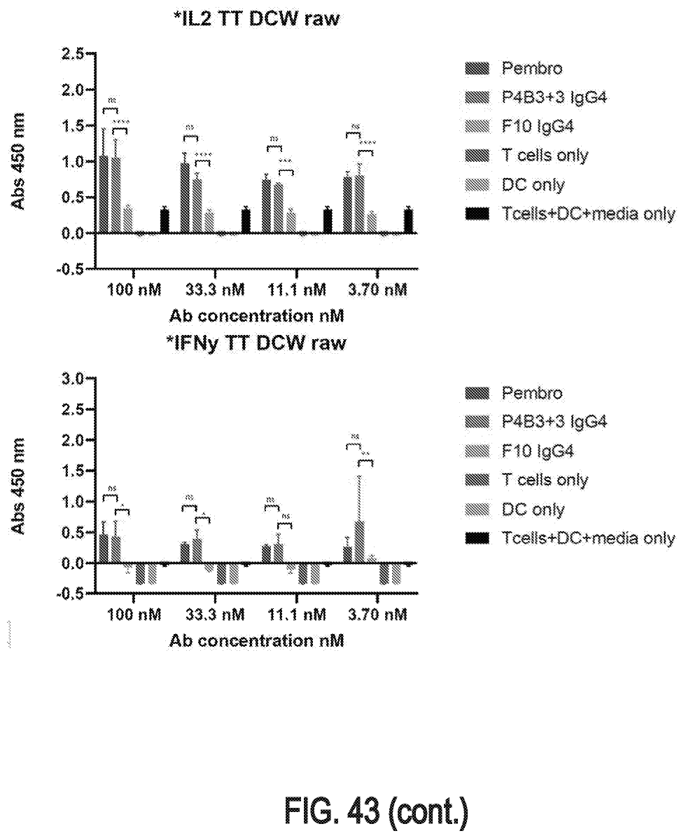

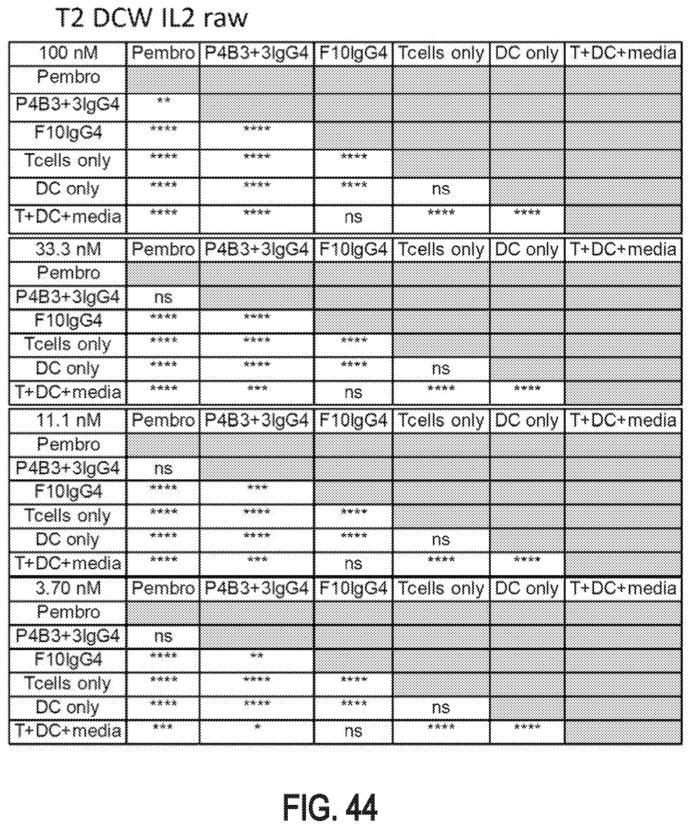

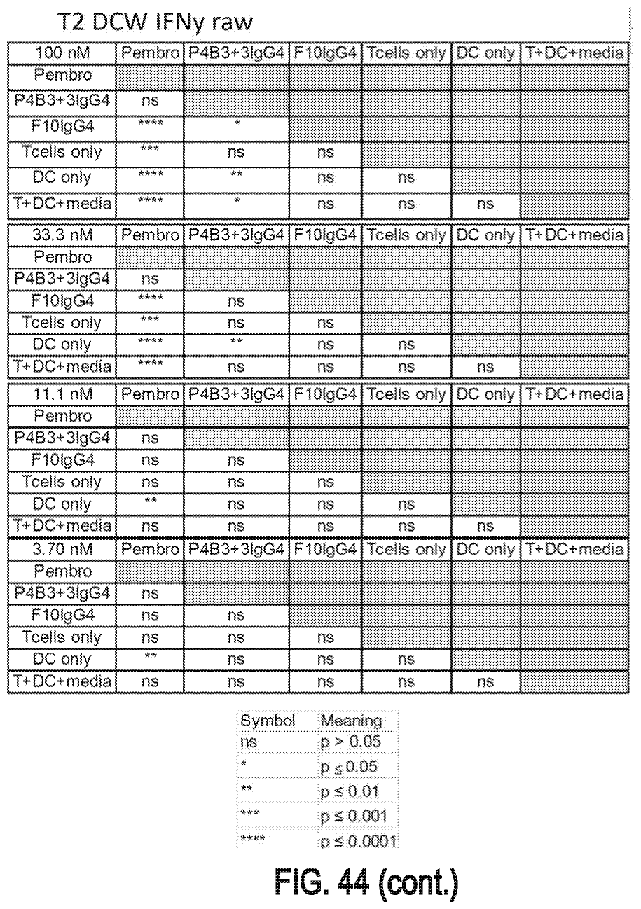

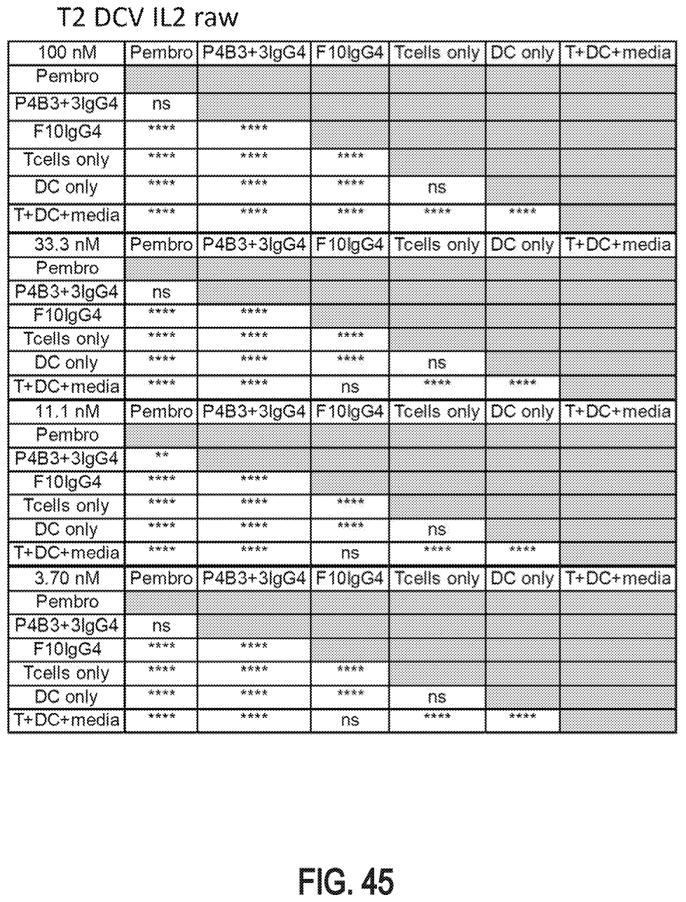

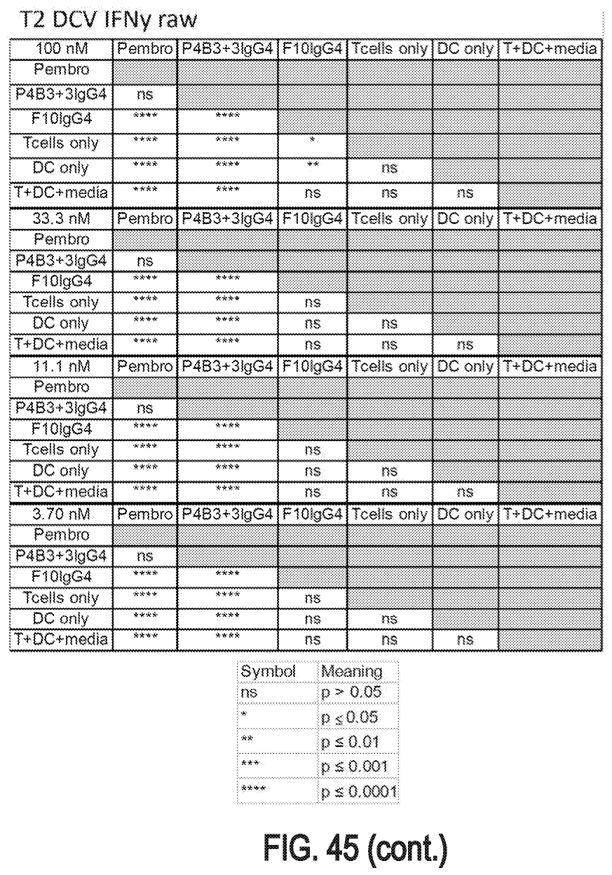

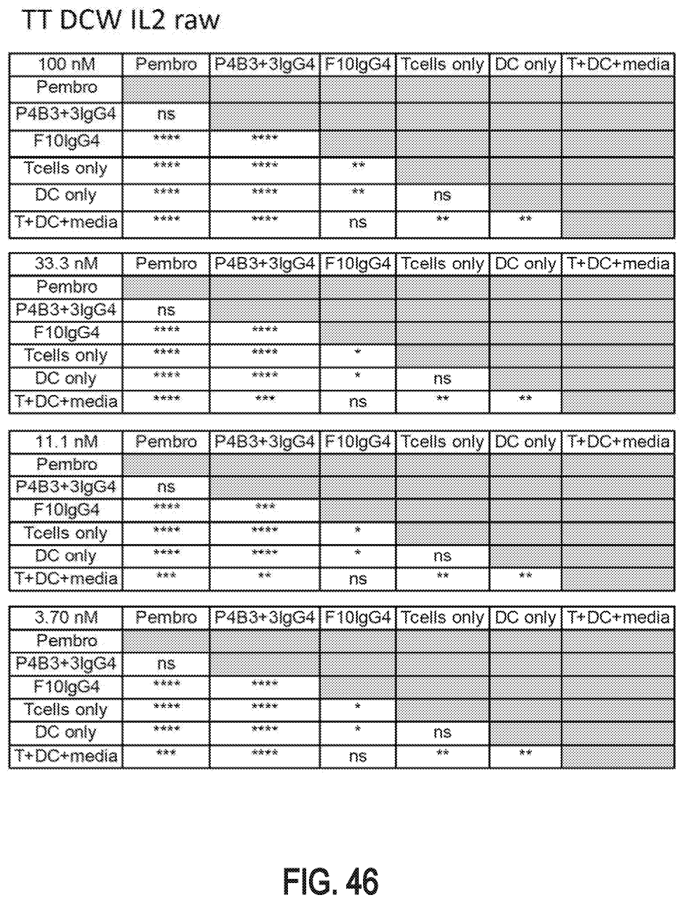

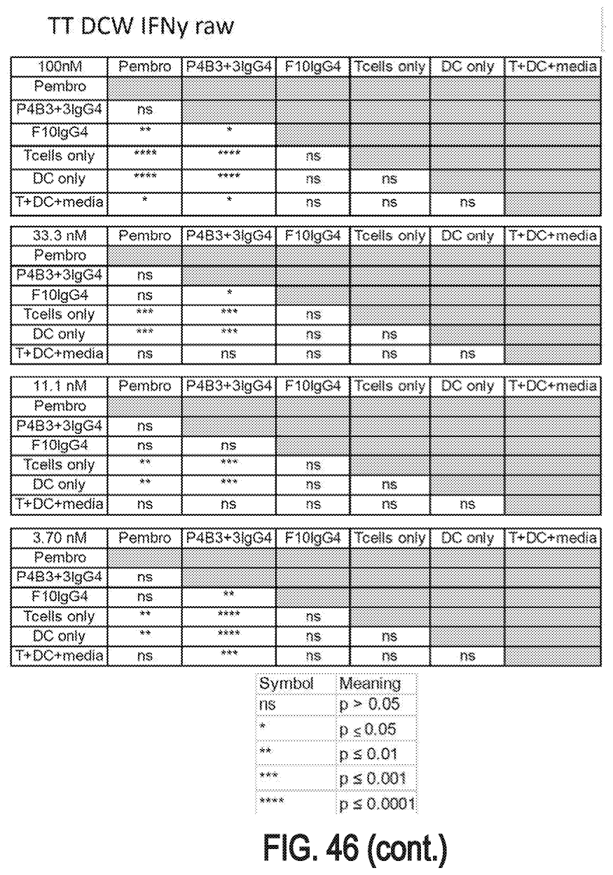

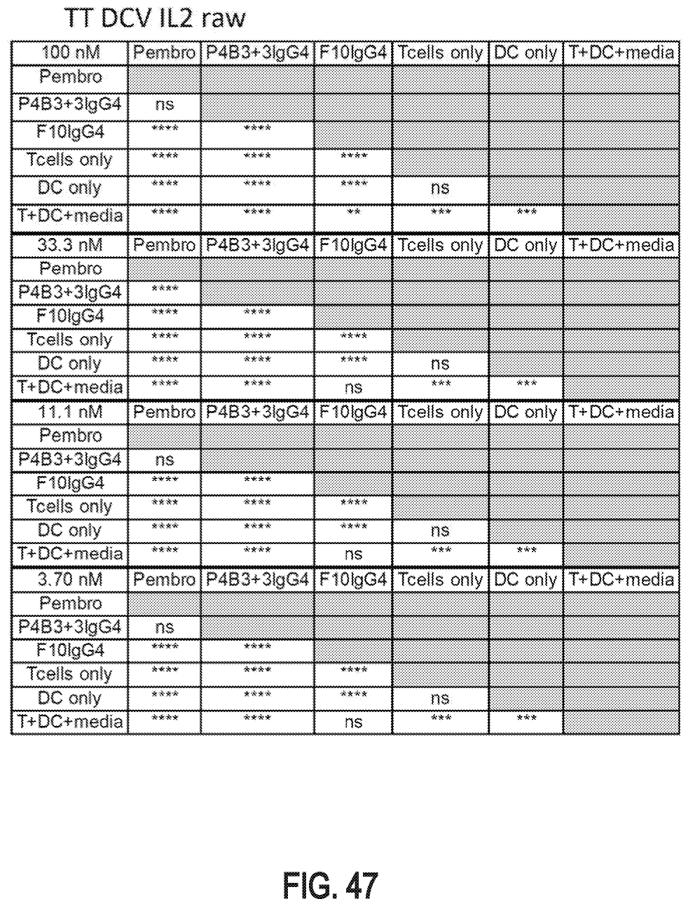

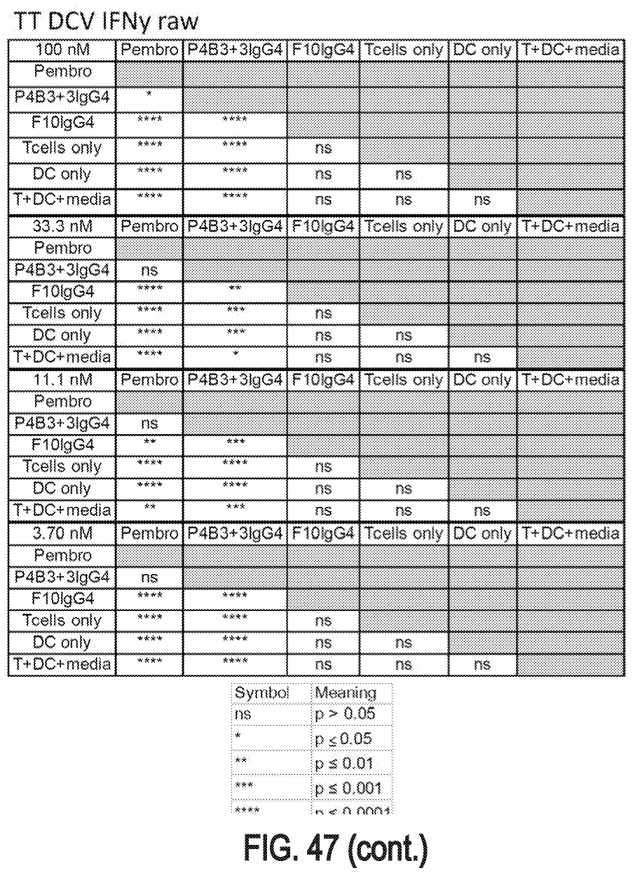

MLR Pembro vs P4-B3 mut+3 IgG4. 2 T cell donors and 2 DC donors were used. Graph titles of FIGS. 42 and 43 denote the cytokine measured, T cell donor, and DC donor. IL2 T2 DCV raw corresponds to an IL2 assay, T cell donor 2, DC donor V.

The P4-B3 mut+3 antibody in sIgG4 format was tested against a commercial preparation of pembrolizumab and F10-sIgG4 (neg control). As shown in FIGS. 42 and 43, addition of either P4-B3 mut+3 or pembrolizumab lead to a significant increase in cytokine production compared to that of F10.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. An isolated monoclonal antibody or antigen-binding fragment thereof that binds to human Programmed cell death 1 (PD-1) protein comprising a heavy chain, light chain, or a combination thereof,
wherein the heavy chain comprises a
CDR1 comprising
(SEQ ID NO: 81)
G-(X₁)-TF-(X₂X₃)-Y-(X₄),
(SEQ ID NO: 82)
G-(X₅)-TF-(X₆X₇X₈)-A,
(SEQ ID NO: 43)
GDSVSSDNYF,
or
(SEQ ID NO: 55)
GYTFNRFG,
CDR2 comprising
(SEQ ID NO: 19)
ISWNSGSI,
(SEQ ID NO: 33)
IYPDDSDT,
(SEQ ID NO: 45)
VYYNGNT,
(SEQ ID NO: 57)
TNPYNGNT,
or
(SEQ ID NO: 69)
ISYDGSNK,
CDR3 comprising
(SEQ ID NO: 21)
ASDYGDKYYYYGMDV,
(SEQ ID NO: 35)
AFWGASGAPVNGFDI,
(SEQ ID NO: 47)
ATETPPTSYFNSGPFDS,
(SEQ ID NO: 59)
ARVVAVNGMDV,
(SEQ ID NO: 71)
ASQTVAGSDY,
or
(SEQ ID NO: 79)
ASDYGDKYSYYGMDV,
or a combination of CDRs thereof; and
wherein the light chain comprises a
CDR1 comprising
(SEQ ID NO: 24)
SSNIGSNT,
(SEQ ID NO: 37)
SSNIGAGYV,
(SEQ ID NO: 49)
SNNVGAHG,
(SEQ ID NO: 61)
SGSIAAYY,
or
(SEQ ID NO: 73)
NIGSKS,
CDR2 comprising
(SEQ ID NO: 83)
(X₉)-DN,
(SEQ ID NO: 84)
(X₁₀)-NN,
or
(SEQ ID NO: 75)
DDS,
CDR3 comprising
(SEQ ID NO: 28)
AAWDGGLNGRGV,
(SEQ ID NO: 41)
AAWDDSLNAPV,
(SEQ ID NO: 53)
SSWDSSLSGYV,
(SEQ ID NO: 65)
QSYDSSNLWV,
or
(SEQ ID NO: 77)
QVWHSVSDQGV,
or a combination of CDRs thereof.

2. The antibody of claim 1, wherein the antibody is fully human or humanized.

3. The antibody of claim 1, wherein the antibody is monospecific, bispecific, or multispecific.

4. The antibody of claim 1, wherein the antibody is a single chain antibody.

5. The antibody of claim 1, wherein the antibody has a binding affinity of at least 1.0×10⁻⁶ M.

6. The antibody or fragment of claim 1, further comprising a heavy chain constant region, a light chain constant region, an Fc region, or a combination thereof.

7. The antibody of claim 1, wherein X₁, X₄, X₅ or X₈ is a non-polar amino acid residue.

8. The antibody of claim 7, wherein X₁, X₄, X₅ or X₈ is tyrosine (Y), phenylalanine (F), or alanine (A).

9. The antibody of claim 1, wherein X₂, X₃, X₄, X₆, X₇ or X₈ is a polar amino acid residue.

10. The antibody of claim 9, wherein X₂, X₃, X₄, X₆, X₇ or X₈ is aspartate (D), threonine (T), serine (S), or tryptophan (W).

11. The antibody of claim 1, wherein X₁ is phenylalanine (F) or tyrosine (Y).

12. The antibody of claim 1, wherein X₂ is aspartate (D), threonine (T), serine (S).

13. The antibody of claim 1, wherein X₃ is aspartate (D), threonine (T), serine (S).

14. The antibody of claim 1, wherein X₄ is alanine (A) or tryptophan (W).

15. The antibody of claim 1, wherein X₅ is phenylalanine (F) or tyrosine (Y).

16. The antibody of claim 1, wherein X₆ is aspartate (D), or serine (S).

17. The antibody of claim 1, wherein X₇ is aspartate (D), or serine (S).

18. The antibody of claim 1, wherein X₈ is phenylalanine (F) or tyrosine (Y).

19. The antibody of claim 1, wherein X₉ is a polar hydrophilic amino acid residue.

20. The antibody of claim 19, wherein X₉ is glutamate (E), asparagine (N), or aspartate (D).

21. The antibody of claim 1, wherein X₁₀ is a polar hydrophilic amino acid residue.

22. The antibody of claim 21, wherein X₁₀ is serine (S) or arginine (R).

23. An antibody composition comprising at least one antibody, wherein the at least one antibody comprises two heavy chains and two light chains, wherein:
the heavy chain CDRs are identical to reference germline CDRs found between residues 27 and 38, residues 56 and 65, and residues 105 and 119 according to IMGT numbering of SEQ ID NO: 1, or between residues 27 and 38, residues 56 and 65, and residues 105 and 119 according to IMGT numbering of SEQ ID NO: 3, or between residues 27 and 38, residues 56 and 65, and residues 105 and 121 according to IMGT numbering of SEQ ID NO: 5, or between residues 27 and 38, residues 56 and 65, and residues 105 and 115 according to IMGT numbering of SEQ ID NO: 7, or between residues 27 and 38, residues 56 and 65, and residues 105 and 114 according to IMGT numbering of SEQ ID NO: 9, or between residues 27 and 38, residues 56 and 65, and residues 105 and 119 according to IMGT numbering of SEQ ID NO: 12, or between residues 27 and 38, residues 56 and 65, and residues 105 and 119 according to IMGT numbering of SEQ ID NO: 13, or between residues 27 and 38, residues 56 and 65, and residues 105 and 119 according to IMGT numbering of SEQ ID NO: 15, except that at least one of the heavy chain CDRs differs by a single amino acid substitution relative to its reference CDR; and
the light chain CDRs are identical to reference germline CDRs found between residues 27 and 38, residues 56 and 65, and residues 105 and 116 according to IMGT numbering of SEQ ID NO: 2, or between residues 27 and 38, residues 56 and 65, and residues 105 and 115 according to IMGT numbering of SEQ ID NO: 4, or between residues 27 and 38, residues 56 and 65, and residues 105 and 115 according to IMGT numbering of SEQ ID NO: 6, or between residues 27 and 38, residues 56 and 65, and residues 105 and 114 according to IMGT numbering of SEQ ID NO: 8, or between residues 27 and 38, residues 56 and 65, and residues 105 and 115 according to IMGT numbering of SEQ ID NO: 10, or between residues 27 and 38, residues 56 and 65, and residues 105 and 116 according to IMGT numbering of SEQ ID NO: 11, except that at least one of the light chain CDRs differs by a single amino acid substitution relative to its reference CDR, and
wherein the antibody composition binds to an epitope that comprises amino residues within the PD-1 face generated by the FCC′ strands but which do not contact the C′D loop of PD-1 comprising non-contiguous amino acids in SEQ ID NO: XX.

24. An isolated antibody or fragment thereof that binds to human Programmed cell death 1 (PD-1) protein comprising:
(a) a VH CDR1 comprising the amino acid sequence of SEQ ID NO: 17, a VH CDR2 comprising the amino acid sequence of SEQ ID NO: 19, a VH CDR3 comprising the amino acid sequence of SEQ ID NO: 21, a VL CDR1 comprising the amino acid sequence of SEQ ID NO: 24, a VL CDR2 comprising the amino acid sequence of SEQ ID NO: 26, and a VL CDR3 comprising the amino acid sequence of SEQ ID NO: 28; or
(b) a VH CDR1 comprising the amino acid sequence of SEQ ID NO: 31, a VH CDR2 comprising the amino acid sequence of SEQ ID NO: 33, a VH CDR3 comprising the amino acid sequence of SEQ ID NO: 35, a VL CDR1 comprising the amino acid sequence of SEQ ID NO: 37, a VL CDR2 comprising the amino acid sequence of SEQ ID NO: 39, and a VL CDR3 comprising the amino acid sequence of SEQ ID NO: 41; or
(c) a VH CDR1 comprising the amino acid sequence of SEQ ID NO: 43, a VH CDR2 comprising the amino acid sequence of SEQ ID NO: 45, a VH CDR3 comprising the amino acid sequence of SEQ ID NO: 47, a VL CDR1 comprising the amino acid sequence of SEQ ID NO: 49, a VL CDR2 comprising the amino acid sequence of SEQ ID NO: 51, and a VL CDR3 comprising the amino acid sequence of SEQ ID NO: 53; or
(d) a VH CDR1 comprising the amino acid sequence of SEQ ID NO: 55, a VH CDR2 comprising the amino acid sequence of SEQ ID NO: 57, a VH CDR3 comprising the amino acid sequence of SEQ ID NO: 59, a VL CDR1 comprising the amino acid sequence of SEQ ID NO: 61, a VL CDR2 comprising the amino acid sequence of SEQ ID NO: 63, and a VL CDR3 comprising the amino acid sequence of SEQ ID NO: 65; or
(e) a VH CDR1 comprising the amino acid sequence of SEQ ID NO: 67, a VH CDR2 comprising the amino acid sequence of SEQ ID NO: 69, a VH CDR3 comprising the amino acid sequence of SEQ ID NO: 71, a VL CDR1 comprising the amino acid sequence of SEQ ID NO: 73, a VL CDR2 comprising the amino acid sequence of SEQ ID NO: 75, and a VL CDR3 comprising the amino acid sequence of SEQ ID NO: 77; or
(f) a VH CDR1 comprising the amino acid sequence of SEQ ID NO: 17, a VH CDR2 comprising the amino acid sequence of SEQ ID NO: 19, a VH CDR3 comprising the amino acid sequence of SEQ ID NO: 21, a VL CDR1 comprising the amino acid sequence of SEQ ID NO: 24, a VL CDR2 comprising the amino acid sequence of SEQ ID NO: 80, and a VL CDR3 comprising the amino acid sequence of SEQ ID NO: 28; or
(g) a VH CDR1 comprising the amino acid sequence of SEQ ID NO: 17, a VH CDR2 comprising the amino acid sequence of SEQ ID NO: 19, a VH CDR3 comprising the amino acid sequence of SEQ ID NO: 79, a VL CDR1 comprising the amino acid sequence of SEQ ID NO: 24, a VL CDR2 comprising the amino acid sequence of SEQ ID NO: 26, and a VL CDR3 comprising the amino acid sequence of SEQ ID NO: 28; or
(h) a VH CDR1 comprising the amino acid sequence of SEQ ID NO: 78, a VH CDR2 comprising the amino acid sequence of SEQ ID NO: 19, a VH CDR3 comprising the amino acid sequence of SEQ ID NO: 21, a VL CDR1 comprising the amino acid sequence of SEQ ID NO: 24, a VL CDR2 comprising the amino acid sequence of SEQ ID NO: 26, and a VL CDR3 comprising the amino acid sequence of SEQ ID NO: 28; or
(i) a VH CDR1 comprising the amino acid sequence of SEQ ID NO: 78, a VH CDR2 comprising the amino acid sequence of SEQ ID NO: 19, a VH CDR3 comprising the amino acid sequence of SEQ ID NO: 21, a VL CDR1 comprising the amino acid sequence of SEQ ID NO: 24, a VL CDR2 comprising the amino acid sequence of SEQ ID NO: 80, and a VL CDR3 comprising the amino acid sequence of SEQ ID NO: 28; or
(j) a VH CDR1 comprising the amino acid sequence of SEQ ID NO: 78, a VH CDR2 comprising the amino acid sequence of SEQ ID NO: 19, a VH CDR3 comprising the amino acid sequence of SEQ ID NO: 79, a VL CDR1 comprising the amino acid sequence of SEQ ID NO: 24, a VL CDR2 comprising the amino acid sequence of SEQ ID NO: 80, and a VL CDR3 comprising the amino acid sequence of SEQ ID NO: 28.

25. An isolated antibody or fragment thereof that binds to human PD-1 protein comprising a heavy chain variable region comprising an amino acid sequence selected from the group consisting of SEQ ID NOS: 1, 3, 5, 7, 9, 12, 13, and 15, and a light chain variable region comprising an amino acid sequence selected from the group consisting of SEQ ID NOS: 2, 4, 6, 8, 10, and 11.

26. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 1, and the light chain comprises an amino acid sequence about 95% identical to SEQ ID NO: 2.

27. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 3, and the light chain comprises an amino acid sequence about 95% identical to SEQ ID NO: 4.

28. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 5, and the light chain comprises an amino acid sequence about 95% identical to SEQ ID NO: 6.

29. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 7, and the light chain comprises an amino acid sequence about 95% identical to SEQ ID NO: 8.

30. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 9, and the light chain comprises an amino acid sequence about 95% identical to SEQ ID NO: 10.

31. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 1, and the light chain comprises an amino acid sequence about 95% identical to SEQ ID NO: 11.

32. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 12, and the light chain comprises an amino acid sequence about 95% identical to SEQ ID NO: 2.

33. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 13, and the light chain comprises an amino acid sequence about 95% identical to SEQ ID NO: 2.

34. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 13, and the light chain comprises an amino acid sequence about 95% identical to SEQ ID NO: 11.

35. An isolated monoclonal antibody or antigen-binding fragment thereof that binds to PD-1 comprising a heavy chain, a light chain, or a combination thereof, wherein the heavy chain comprises an amino acid sequence about 95% identical to SEQ ID NO: 15, and the light chain comprises an amino acid sequence about 95% identical to SEQ ID NO: 11.

36. An isolated bispecific antibody comprising a fragment of claim 1, 23, 24, 25, 26, 27, 28, 29, 30, 31, 32, 33, 34, or 35 and a second antigen-binding fragment having specificity to a molecule on an immune cell.

37. The bispecific antibody of claim 36, wherein the molecule is selected from the group consisting of B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-1BB, TIGIT, VISTA, HEVM, BTLA, and KIR.

38. The bispecific antibody of claim 36, wherein the fragment and the second fragment each is independently selected from a Fab fragment, a single-chain variable fragment (scFv), or a single-domain antibody.

39. The bispecific antibody of claim 36, further comprising a Fc fragment.

40. A nucleic acid encoding the antibody according to any one of claims 1-35.

41. A nucleic acid encoding the bispecific antibody according to any one of claims 36-39.

42. A pharmaceutical composition comprising the antibody or fragment thereof according to any one of claims 1-35, and a pharmaceutically acceptable carrier or excipient.

43. The pharmaceutical composition of claim 42, further comprising at least one additional therapeutic agent.

44. The pharmaceutical composition of claim 43, wherein the therapeutic agent is a toxin, a radiolabel, a siRNA, a small molecule, or a cytokine.

45. A pharmaceutical composition comprising the bispecific antibody according to any one of claims 36-39, and a pharmaceutically acceptable carrier or excipient.

46. The pharmaceutical composition of claim 45, further comprising at least one additional therapeutic agent.

47. The pharmaceutical composition of claim 46, wherein the therapeutic agent is a toxin, a radiolabel, a siRNA, a small molecule, or a cytokine.

48. An isolated cell comprising one or more polynucleotide(s) encoding the antibody or fragment thereof of any one of claims 1-35.

49. An isolated cell comprising one or more polynucleotide(s) encoding the bispecific antibody or fragment thereof of any one of claims 36-39.

50. A vector comprising the nucleic acid of claims 40 or 41.

51. A cell comprising the vector of claim 50.

52. A kit comprising: the at least one antibody composition of claim 42 or 45; a syringe, needle, or applicator for administration of the at least one antibody to a subject; and instructions for use.

53. An engineered cell comprising a chimeric antigen receptor, wherein the chimeric antigen receptor comprises an extracellular ligand binding domain that is specific for an antigen on the surface of a cancer cell, wherein the antigen comprises PD-1.

54. The engineered cell of claim 53, wherein the extracellular ligand binding domain comprises an antibody or fragment thereof.

55. The engineered cell of claim 53, wherein the antibody comprises a VH and/or VL according to Tables 1-11, or any combination thereof.

56. The engineered cell of claim 54, wherein the antibody comprises a CDR1, CDR2, and/or CDR3 of Table 12, or any combination thereof.

57. The engineered cell of claim 53, wherein the engineered cell comprises a T cell, an NK cell, or an NKT cell.

58. The engineered cell of claim 57, wherein the T cell is CD4+, CD8+, CD3+ panT cells, or any combination thereof.

59. A method of treating cancer in a subject, the method comprising administering to a subject in need thereof a therapeutically effective amount of a composition comprising an antibody according to any one of claims 1-39, the pharmaceutical composition according to any one of claims 42-46, or the CAR composition according to any one of claims 53-58.

60. The method of claim 59, wherein the cancer expresses PD-1.

61. The method of claim 59, wherein the cancer comprises non-small-cell lung cancer, melanoma, ovarian cancer, lymphoma, or renal-cell cancer.

62. The method of claim 59, further comprising administering to the subject a chemotherapeutic agent.
